SEQUENCE LISTING (357 sequences)

SEQ ID NO: 1 - DNA, 19 bp, Homo sapiens
gcggagcagggttacaacg

SEQ ID NO: 2 - DNA, 19 bp, Homo sapiens
tggggcagtgtggaaagaa

SEQ ID NO: 3 - DNA, 21 bp, Homo sapiens
agctgcgttgcgtttgtttac

SEQ ID NO: 4 - DNA, 23 bp, Homo sapiens
gcgaacacttgcagattactgat

SEQ ID NO: 5 - DNA, 16 bp, Homo sapiens
agacaatgggaactgg

SEQ ID NO: 6 - DNA, 16 bp, Homo sapiens
gtttacagaccacgca

SEQ ID NO: 7 - DNA, 21 bp, Homo sapiens
cgggaaggaaatttgtcttga

SEQ ID NO: 8 - DNA, 21 bp, Homo sapiens
agcttaagcggcaaacatagg

SEQ ID NO: 9 - DNA, 21 bp, Homo sapiens
aaaatcaagtggggcgatgct

SEQ ID NO: 10 - DNA, 20 bp, Homo sapiens
gccaggggtgctaagcagtt

SEQ ID NO: 11 - DNA, 30 bp, Homo sapiens
agtcatagtcccttccaacaccctcctcct

SEQ ID NO: 12 - DNA, 30 bp, Homo sapiens
tcatccttgtctcctctagtcttctctttt

SEQ ID NO: 13 - DNA, 775 bp, Homo sapiens
acaatagtcaataataacttaattgtacattttaaaataactcataaagaatgtaactggattgttcgtaacccaaaggataaatgcttgaggggatggatacctaattctcaatatgcttatttcacattgcatgcctgtatcaaaacagctcatgtaccccatacatatatacagctactatgtacccacaacattttcttaagaataagaaaaataataaaataatttaaaacaacattttaatggcttaattcacatgagaggttgttggtgttggctgctggttgtgatttctactggggcacctgcgttcttctcctactcttttaaatataattatttaacttctcactaataagcagaaatgaggaatggcgggagagaaggagagtaggcaagggaaggggagggaagagaagaagaaggaaaaggagagaaggaagaaaatagaaaggaaaaaacaggcaagtcatagtcccttccaacaccctcctcctytcatccttgtctcctctagtcttctcttttctagttcttcccctcacctcttctcccctttcttccactcttcttactggaaaatatttgttctgtgggaaaactgagtatatttgcagcagaaaaccttaacttagactgaaagttcctcatctttataatttccagatcataaggatcctacaaagataataggcatgtaacaggattatcgattaatatcaataaattaatatttgattgatgaatacttatacaaaggaaaatacatcat

SEQ ID NO: 14 - DNA, 30 bp, Homo sapiens
ctggcttcccatggaaatcactctggccca

SEQ ID NO: 15 - DNA, 30 bp, Homo sapiens
tgcccatgtctggttcctatataggcatct

SEQ ID NO: 16 - DNA, 201 bp, Homo sapiens
atagaaagtccccactagaaatgggctgttgggttgcccaacctaatcaggaagcaatatgtcacttaaactggcttcccatggaaatcactctggcccaytgcccatgtctggttcctatataggcatcttggatgttggctttgtacaaacgtggatggtccaaaagtcctctaaggttgagtatgacagcaccaagaa

SEQ ID NO: 17 - DNA, 30 bp, Homo sapiens
aattctgattatgttatactgtgttgaatg

SEQ ID NO: 18 - DNA, 30 bp, Homo sapiens
taacagtgtgggtatgtcattctaaattga

SEQ ID NO: 19 - DNA, 201 bp, Homo sapiens
tattgaccaaataacacagaccaaatagtttctgtgacattgagattgtaggatgtaaatgacaaggggaaattctgattatgttatactgtgttgaatgwtaacagtgtgggtatgtcattctaaattgatcttttccaacaaaccatagaagcctttaactctgtgatttagtatttggcataggccttttcaaatgtt

SEQ ID NO: 20 - DNA, 30 bp, Homo sapiens
tcagctttcttctccataaaactattcaga

SEQ ID NO: 21 - DNA, 30 bp, Homo sapiens
gaacaaaaaggatagctgtgaaaactttta

SEQ ID NO: 22 - DNA, 30 bp, Homo sapiens; identical to SEQ ID NO: 20

SEQ ID NO: 23 - DNA, 30 bp, Homo sapiens; identical to SEQ ID NO: 21

SEQ ID NO: 24 - DNA, 30 bp, Homo sapiens; identical to SEQ ID NO: 20

SEQ ID NO: 25 - DNA, 30 bp, Homo sapiens; identical to SEQ ID NO: 21

SEQ ID NO: 26 - DNA, 30 bp, Homo sapiens; identical to SEQ ID NO: 20

SEQ ID NO: 27 - DNA, 30 bp, Homo sapiens; identical to SEQ ID NO: 21

SEQ ID NO: 28 - DNA, 201 bp, Homo sapiens
cattcttctgccacaaactttctgtatgatctcaaacaatttactatttatttaaatgaaaatactaatatcagctttcttctccataaaactattcagargaacaaaaaggatagctgtgaaaacttttaaaaatattttttcttcttttatgttttacattgatgtaaaattttcacagtgagatgtacatatcttaag

SEQ ID NO: 29 - DNA, 30 bp, Homo sapiens
aactccagaggatgttcttttaaccagatc

SEQ ID NO: 30 - DNA, 30 bp, Homo sapiens
tatctatcttttaaaaaattgtttagttca

SEQ ID NO: 31 - DNA, 30 bp, Homo sapiens; identical to SEQ ID NO: 29

SEQ ID NO: 32 - DNA, 30 bp, Homo sapiens; identical to SEQ ID NO: 30

SEQ ID NO: 33 - DNA, 30 bp, Homo sapiens; identical to SEQ ID NO: 29

SEQ ID NO: 34 - DNA, 30 bp, Homo sapiens; identical to SEQ ID NO: 30

SEQ ID NO: 35 - DNA, 30 bp, Homo sapiens; identical to SEQ ID NO: 29

SEQ ID NO: 36 - DNA, 30 bp, Homo sapiens; identical to SEQ ID NO: 30

SEQ ID NO: 37 - DNA, 30 bp, Homo sapiens; identical to SEQ ID NO: 29

SEQ ID NO: 38 - DNA, 30 bp, Homo sapiens; identical to SEQ ID NO: 30

SEQ ID NO: 39 - DNA, 30 bp, Homo sapiens; identical to SEQ ID NO: 29

SEQ ID NO: 40 - DNA, 30 bp, Homo sapiens; identical to SEQ ID NO: 30

SEQ ID NO: 41 - DNA, 30 bp, Homo sapiens; identical to SEQ ID NO: 29

SEQ ID NO: 42 - DNA, 30 bp, Homo sapiens; identical to SEQ ID NO: 30

SEQ ID NO: 43 - DNA, 30 bp, Homo sapiens; identical to SEQ ID NO: 29

SEQ ID NO: 44 - DNA, 30 bp, Homo sapiens; identical to SEQ ID NO: 30

SEQ ID NO: 45 - DNA, 748 bp, Homo sapiens
ggaaaccactaaacataatgagtgagatgcctgataccctataattattatagtatatacagtatgctatttatgtatcaagttgtattacatgtgtgaataatactatgttatatgtaatgtaatctcttcattagagaaagaagagttgaggaaagcactgtggtgtggtggttaagagcacaatttgaaattcccactctgttacctacttagttatctgaaagtgggcaagtattttaaactctataaacttgatttttctcttctgtaacactggggaggataataataatcataagaaataattactaacatttatgtgtggcttatttatgacacacattattttaaataccttaaatttattaacttttttaatccttccataaactctatgagggaggcaatattatccccattttagggatgaagaacctaaggtacagagaaattaagcaacatgacctgatatacacagctaacaagtggcagagccaggttacctcattattcaaactccagaggatgttcttttaaccagatcrtatctatcttttaaaaaattgtttagttcatgcatggaaaacacttagtattcctggcatattaaaatgattaataaattggaactgttatcatactacattaagaatgacaccgatcatcaatttagtaacttttgttgttcaaactgtgaaataccaagtatacaaaaaagagaggaaaattaaagctttttttcccc

SEQ ID NO: 46 - DNA, 30 bp, Homo sapiens
ctgttcaacaccaaaatacattgctgaaaa

SEQ ID NO: 47 - DNA, 30 bp, Homo sapiens
gtttattggcttcactggacaccttttttc

SEQ ID NO: 48 - DNA, 30 bp, Homo sapiens; identical to SEQ ID NO: 46

SEQ ID NO: 49 - DNA, 30 bp, Homo sapiens; identical to SEQ ID NO: 47

SEQ ID NO: 50 - DNA, 30 bp, Homo sapiens
  misc_feature (10)..(10): n is a, c, g, or t
gtggtgggcnccggaatacattgctgaaaa

SEQ ID NO: 51 - DNA, 30 bp, Homo sapiens; identical to SEQ ID NO: 47

SEQ ID NO: 52 - DNA, 30 bp, Homo sapiens; identical to SEQ ID NO: 46

SEQ ID NO: 53 - DNA, 30 bp, Homo sapiens; identical to SEQ ID NO: 47

SEQ ID NO: 54 - DNA, 30 bp, Homo sapiens; identical to SEQ ID NO: 46

SEQ ID NO: 55 - DNA, 30 bp, Homo sapiens; identical to SEQ ID NO: 47

SEQ ID NO: 56 - DNA, 30 bp, Homo sapiens; identical to SEQ ID NO: 46

SEQ ID NO: 57 - DNA, 30 bp, Homo sapiens; identical to SEQ ID NO: 47

SEQ ID NO: 58 - DNA, 30 bp, Homo sapiens; identical to SEQ ID NO: 46

SEQ ID NO: 59 - DNA, 30 bp, Homo sapiens; identical to SEQ ID NO: 47

SEQ ID NO: 60 - DNA, 30 bp, Homo sapiens; identical to SEQ ID NO: 46

SEQ ID NO: 61 - DNA, 30 bp, Homo sapiens; identical to SEQ ID NO: 47

SEQ ID NO: 62 - DNA, 30 bp, Homo sapiens; identical to SEQ ID NO: 46

SEQ ID NO: 63 - DNA, 30 bp, Homo sapiens; identical to SEQ ID NO: 47

SEQ ID NO: 64 - DNA, 30 bp, Homo sapiens; identical to SEQ ID NO: 46

SEQ ID NO: 65 - DNA, 30 bp, Homo sapiens; identical to SEQ ID NO: 47

SEQ ID NO: 66 - DNA, 30 bp, Homo sapiens; identical to SEQ ID NO: 46

SEQ ID NO: 67 - DNA, 30 bp, Homo sapiens
gtttattggcttcactggacaccttttttt

SEQ ID NO: 68 - DNA, 995 bp, Homo sapiens
  misc_feature (25)..(25): n is a, c, g, or t
tcccgttggctttcataaacatgtntaaaattatttctgcctcactagccttagtttcagttgatgaatgtatttcttatttttctccacctcatttattgttttctttaaattagagcttagcagtacagcatatttaattggaatgatttttctccctgtcacaacatagaaatttctagtcttaagcaagcttaaaggaagggtgtaagctgagagttcggagtcttctgagagttgagatgtggagttaattgcagagataagctcagcaggacacaaaatgaggggagcagacttctttctctttcctcctgtggggtgactttatagcttttcttcattattgagattgagatattgtgaattcgcaagtcatatacattttcaaacataagatcaaagccatttccagaacaagatataaaggttttttttttaacactattcatagtatatttcattttctgttcaacaccaaaatacattgctgaaaaygtttattggcttcactggacaccttttttccccctacttaacgttatccagatttctttcctcgttcattagtaagatttcctaaaagttcctaatcgttttaagaaaactgtatgaaaagttttctgaagtaatgatgcttttgagtgagttgataccaggaaaaaggtaggctctttcctaccaggaaaccaactggtgggttaataccaggaaagaggtgactccagtatcatggagtcattgtcttaatcaacaaatgtggcttgtcacgaaagacacatcgtcggcaaaattcgtgaggaagtagtaacagatttcttgagtcatcaaaatttcttaaggatatcagtcagcatttgatataaaacaatattttgctagtttttattaactcaaaaaatattgattaagcatcagttgattaagcctgaacttcctaacaggttttacgaagttctctttgcatgacttggtcatttaattatttatttttt

SEQ ID NO: 69 - DNA, 30 bp, Homo sapiens
agttatctttgcttgccaaagaaagcaaat

SEQ ID NO: 70 - DNA, 30 bp, Homo sapiens
ttcttttcatgaatgttgataaatattggg

SEQ ID NO: 71 - DNA, 401 bp, Homo sapiens
cctgtgtaagaactccgttcaggaaggtaagaactagactgaaatactacatgcattttctttgtatgatttataacaatacgccctgccttttctatactgcagtttttaggcttaatagaaactttaggagcacctgaaggcacaaacatgtggcccttgaaagagaaagttatctttgcttgccaaagaaagcaaatsttcttttcatgaatgttgataaatattgggattggtttggagcatgtaagtaaaataagagccaattaccgtcaaaggttgtctttggattttagcttattggattctttctgccttcaatctatattatcttttgtaatttcatctagaaactacaaatgaaactcatcctcctttatattacaaaagaagctacttgt

SEQ ID NO: 72 - DNA, 30 bp, Homo sapiens
atactcaaccttagaggacttttggaccat

SEQ ID NO: 73 - DNA, 30 bp, Homo sapiens
cacgtttgtacaaagccaacatccaagatg

SEQ ID NO: 74 - DNA, 30 bp, Homo sapiens
catcttggatgttggctttgtacaaacgtg

SEQ ID NO: 75 - DNA, 30 bp, Homo sapiens
atggtccaaaagtcctctaaggttgagtat

SEQ ID NO: 76 - DNA, 601 bp, Homo sapiens
aatcatttatactgtattagtttatattacattatactgtggtataatgactaaaataggattgctttagacagacagttaagccttcaggctgtcaggatgctgataccattggatgaattattctattttagttgggaacaaaaataacagagtttttctctaccacctggatacagtgcaggtaagacaaatggcttttgtttaaactgatgataaagaagcaactactgttaaagtgctgtgtcttcagtggttcttggtgctgtcatactcaaccttagaggacttttggaccatscacgtttgtacaaagccaacatccaagatgcctatataggaaccagacatgggcagtgggccagagtgatttccatgggaagccagtttaagtgacatattgcttcctgattaggttgggcaacccaacagcccatttctagtggggactttctatcatgtgcttgtcagctaaagaagtgttttttttcttcttgcattccataaacaagctacagtgaattgattcagagtgcaaaccaggtgagatatatacatttataagaccgtcaggaggaactggcaatcttttctcatgatc

SEQ ID NO: 77 - DNA, 30 bp, Homo sapiens
ttcattattgagattgagatattgtgaatt

SEQ ID NO: 78 - DNA, 30 bp, Homo sapiens
gcaagtcatatacattttcaaacataagat

SEQ ID NO: 79 - DNA, 30 bp, Homo sapiens; identical to SEQ ID NO: 77

SEQ ID NO: 80 - DNA, 30 bp, Homo sapiens; identical to SEQ ID NO: 78

SEQ ID NO: 81 - DNA, 30 bp, Homo sapiens; identical to SEQ ID NO: 77

SEQ ID NO: 82 - DNA, 30 bp, Homo sapiens; identical to SEQ ID NO: 78

SEQ ID NO: 83 - DNA, 30 bp, Homo sapiens; identical to SEQ ID NO: 77

SEQ ID NO: 84 - DNA, 30 bp, Homo sapiens; identical to SEQ ID NO: 78

SEQ ID NO: 85 - DNA, 30 bp, Homo sapiens; identical to SEQ ID NO: 77

SEQ ID NO: 86 - DNA, 30 bp, Homo sapiens; identical to SEQ ID NO: 78

SEQ ID NO: 87 - DNA, 30 bp, Homo sapiens; identical to SEQ ID NO: 77

SEQ ID NO: 88 - DNA, 30 bp, Homo sapiens; identical to SEQ ID NO: 78

SEQ ID NO: 89 - DNA, 30 bp, Homo sapiens; identical to SEQ ID NO: 77

SEQ ID NO: 90 - DNA, 30 bp, Homo sapiens; identical to SEQ ID NO: 78

SEQ ID NO: 91 - DNA, 30 bp, Homo sapiens; identical to SEQ ID NO: 77

SEQ ID NO: 92 - DNA, 30 bp, Homo sapiens; identical to SEQ ID NO: 78

SEQ ID NO: 93 - DNA, 25 bp, Homo sapiens
atgtttgaaaatgtatatgacttgc

SEQ ID NO: 94 - DNA, 25 bp, Homo sapiens
aattcacaatatctcaatctcaata

SEQ ID NO: 95 - DNA, 30 bp, Homo sapiens; identical to SEQ ID NO: 77

SEQ ID NO: 96 - DNA, 30 bp, Homo sapiens; identical to SEQ ID NO: 78

SEQ ID NO: 97 - DNA, 30 bp, Homo sapiens; identical to SEQ ID NO: 77

SEQ ID NO: 98 - DNA, 30 bp, Homo sapiens; identical to SEQ ID NO: 78

SEQ ID NO: 99 - DNA, 30 bp, Homo sapiens; identical to SEQ ID NO: 77

SEQ ID NO: 100 - DNA, 30 bp, Homo sapiens; identical to SEQ ID NO: 78

SEQ ID NO: 101 - DNA, 996 bp, Homo sapiens
  misc_feature (26)..(26): n is a, c, g, or t
  misc_feature (153)..(153): n is a, c, g, or t
gaagacaactctggtatgaggagttnttctgccttattttaagcttttctattatataaagcccttgtaacttgtgaaagactctagaacacccccaactttgtggatgtgtcttcccaggtctgtcttcccgttggctttcataaacatgtntaaaattatttctgcctcactagccttagtttcagttgatgaatgtatttcttatttttctccacctcatttattgttttctttaaattagagcttagcagtacagcatatttaattggaatgatttttctccctgtcacaacatagaaatttctagtcttaagcaagcttaaaggaagggtgtaagctgagagttcggagtcttctgagagttgagatgtggagttaattgcagagataagctcagcaggacacaaaatgaggggagcagacttctttctctttcctcctgtggggtgactttatagcttttcttcattattgagattgagatattgtgaattygcaagtcatatacattttcaaacataagatcaaagccatttccagaacaagatataaaggttttttttttaacactattcatagtatatttcattttctgttcaacaccaaaatacattgctgaaaatgtttattggcttcactggacaccttttttccccctacttaacgttatccagatttctttcctcgttcattagtaagatttcctaaaagttcctaatcgttttaagaaaactgtatgaaaagttttctgaagtaatgatgcttttgagtgagttgataccaggaaaaaggtaggctctttcctaccaggaaaccaactggtgggttaataccaggaaagaggtgactccagtatcatggagtcattgtcttaatcaacaaatgtggcttgtcacgaaagacacatcgtcggcaaaattcgtgaggaagtagtaacagatttcttgagtcatcaaaatttcttaaggatatcagtcagcatttgatataaaa

SEQ ID NO: 102 - DNA, 30 bp, Homo sapiens
tcccacagaacaaatattttccagtaagaa

SEQ ID NO: 103 - DNA, 30 bp, Homo sapiens
agtggaagaaaggggagaagaggtgagggg

SEQ ID NO: 104 - DNA, 401 bp, Homo sapiens
tgtattttcctttgtataagtattcatcaatcaaatattaatttattgatattaatcgataatcctgttacatgcctattatctttgtaggatccttatgatctggaaattataaagatgaggaactttcagtctaagttaaggttttctgctgcaaatatactcagttttcccacagaacaaatattttccagtaagaaragtggaagaaaggggagaagaggtgaggggaagaactagaaaagagaagactagaggagacaaggatgagaggaggagggtgttggaagggactatgacttgcctgttttttcctttctattttcttccttctctccttttccttcttcttctcttccctccccttcccttgcctactctccttctctcccgccattcct

SEQ ID NO: 105 - DNA, 30 bp, Homo sapiens
aactagaaaagagaagactagaggagacaa

SEQ ID NO: 106 - DNA, 30 bp, Homo sapiens
gatgagaggaggagggtgttggaagggact

SEQ ID NO: 107 - DNA, 30 bp, Homo sapiens
agtcccttccaacaccctcctcctctcatc

SEQ ID NO: 108 - DNA, 30 bp, Homo sapiens
ttgtctcctctagtcttctcttttctagtt

SEQ ID NO: 109 - DNA, 601 bp, Homo sapiens
ttctgtcaattacatagtacctatcaaaatatatgatgtattttcctttgtataagtattcatcaatcaaatattaatttattgatattaatcgataatcctgttacatgcctattatctttgtaggatccttatgatctggaaattataaagatgaggaactttcagtctaagttaaggttttctgctgcaaatatactcagttttcccacagaacaaatattttccagtaagaagagtggaagaaaggggagaagaggtgaggggaagaactagaaaagagaagactagaggagacaargatgagaggaggagggtgttggaagggactatgacttgcctgttttttcctttctattttcttccttctctccttttccttcttcttctcttccctccccttcccttgcctactctccttctctcccgccattcctcatttctgcttattagtgagaagttaaataattatatttaaaagagtaggagaagaacgcaggtgccccagtagaaatcacaaccagcagccaacaccaacaacctctcatgtgaattaagccattaaaatgttgttttaaattattttattatttttcttatt

SEQ ID NO: 110 - DNA, 30 bp, Homo sapiens
catatcccattgctttccacaaatatttta

SEQ ID NO: 111 - DNA, 30 bp, Homo sapiens
gcctgacttttaaaaaataaataattaaat

SEQ ID NO: 112 - DNA, 401 bp, Homo sapiens
ttttaactgaataagtttttaaaaacatcttttaaccagttttgtacactcagtttatcatcatctttactgtgtttctcaacgtggaaatggaagggaatgatgcaaatgacagtaacagcaattaacagcagcagttgtcatcatagcagcaagagtagagtatttttcatatcccattgctttccacaaatattttargcctgacttttaaaaaataaataattaaatgaccaagtcatgcaaagagaacttcgtaaaacctgttaggaagttcaggcttaatcaactgatgcttaatcaatattttttgagttaataaaaactagcaaaatattgttttatatcaaatgctgactgatatccttaagaaattttgatgactcaagaaatctgttact

SEQ ID NO: 113 - DNA, 30 bp, Homo sapiens
gtggtagaaacaagtagcttcttttgtaat

SEQ ID NO: 114 - DNA, 30 bp, Homo sapiens
taaaggaggatgagtttcatttgtagtttc

SEQ ID NO: 115 - DNA, 401 bp, Homo sapiens
ttaccactgaatagtaggaatgtggagaaatgatgaagagaaatttgtgttttatttcaaagacatctgcagcatacgaatcttttgcaataaattatataatttttctgttaaattaaaaataaaaagaaaaataaatatgctgggtcctactttttaggtattcttaggtggtagaaacaagtagcttcttttgtaatrtaaaggaggatgagtttcatttgtagtttctagatgaaattacaaaagataatatagattgaaggcagaaagaatccaataagctaaaatccaaagacaacctttgacggtaattggctcttattttacttacatgctccaaaccaatcccaatatttatcaacattcatgaaaagaacatttgctttctttggcaagca

SEQ ID NO: 116 - DNA, 30 bp, Homo sapiens
aagaaggatttgggaaatgtgcgttaaaaa

SEQ ID NO: 117 - DNA, 30 bp, Homo sapiens
gttctgtgtgaggcagagaagtcatctatg

SEQ ID NO: 118 - DNA, 30 bp, Homo sapiens
cataaatgacttctctgcctcacacagaac

SEQ ID NO: 119 - DNA, 30 bp, Homo sapiens
tttttaacgcacatttcccaaatccttctt

SEQ ID NO: 120 - DNA, 30 bp, Homo sapiens; identical to SEQ ID NO: 118

SEQ ID NO: 121 - DNA, 30 bp, Homo sapiens; identical to SEQ ID NO: 119

SEQ ID NO: 122 - DNA, 30 bp, Homo sapiens; identical to SEQ ID NO: 118

SEQ ID NO: 123 - DNA, 30 bp, Homo sapiens; identical to SEQ ID NO: 119

SEQ ID NO: 124 - DNA, 30 bp, Homo sapiens; identical to SEQ ID NO: 118

SEQ ID NO: 125 - DNA, 30 bp, Homo sapiens; identical to SEQ ID NO: 119

SEQ ID NO: 126 - DNA, 30 bp, Homo sapiens
catagatgacttctctgcctcacacagaac

SEQ ID NO: 127 - DNA, 30 bp, Homo sapiens; identical to SEQ ID NO: 119

SEQ ID NO: 128 - DNA, 25 bp, Homo sapiens
ggatttgggaaatgtgcgttaaaaa

SEQ ID NO: 129 - DNA, 25 bp, Homo sapiens
gttctgtgtgaggcagagaagtcat

SEQ ID NO: 130 - DNA, 30 bp, Homo sapiens; identical to SEQ ID NO: 116

SEQ ID NO: 131 - DNA, 30 bp, Homo sapiens; identical to SEQ ID NO: 117

SEQ ID NO: 132 - DNA, 30 bp, Homo sapiens; identical to SEQ ID NO: 116

SEQ ID NO: 133 - DNA, 30 bp, Homo sapiens; identical to SEQ ID NO: 117

SEQ ID NO: 134 - DNA, 30 bp, Homo sapiens; identical to SEQ ID NO: 116

SEQ ID NO: 135 - DNA, 30 bp, Homo sapiens; identical to SEQ ID NO: 117

SEQ ID NO: 136 - DNA, 601 bp, Homo sapiens
tgtcctcccattttcaccccatgcttcacctctgggcattagacagtaaaacctcaattcacatctgaacaattcttgtcaactcatagttatacagtaagcacatttggtttcagcccttacagaaaaacttttgatggctagtattttaaatttattgattaagatactttaatgaacagactcaacattgtactatccttagcatttttggtactaattgcttttatgattgatttgcttgtgctagtctatttccttcatacttgaaagaaggatttgggaaatgtgcgttaaaaaygttctgtgtgaggcagagaagtcatctatgccttaattttatagatagtggtcctccaatgtctctagatattttcttttaaaatagctaacataaaatgtgctttgagtcttagataatttgcttgcttccttcatgactctgctatttataagatgacttaaagaaaaaagagctaaaataacttagcactccccaacacacacatatacaaacccacaatgacaatgtaagattaataaaaaggctgtcaagttattttaaaaacaaggaacaaaataactcttctctaattctagc

SEQ ID NO: 137 - DNA, 30 bp, Homo sapiens
tagcagaagaaactcacgctaacatacaga

SEQ ID NO: 138 - DNA, 30 bp, Homo sapiens
ttatcagaaacaataaattaatattttaag

SEQ ID NO: 139 - DNA, 30 bp, Homo sapiens
cttaaaatattaatttattgtttctgataa

SEQ ID NO: 140 - DNA, 30 bp, Homo sapiens
tctgtatgttagcgtgagtttcttctgcta

SEQ ID NO: 141 - DNA, 30 bp, Homo sapiens; identical to SEQ ID NO: 139

SEQ ID NO: 142 - DNA, 30 bp, Homo sapiens; identical to SEQ ID NO: 140

SEQ ID NO: 143 - DNA, 30 bp, Homo sapiens; identical to SEQ ID NO: 139

SEQ ID NO: 144 - DNA, 30 bp, Homo sapiens; identical to SEQ ID NO: 140

SEQ ID NO: 145 - DNA, 30 bp, Homo sapiens; identical to SEQ ID NO: 139

SEQ ID NO: 146 - DNA, 30 bp, Homo sapiens; identical to SEQ ID NO: 140

SEQ ID NO: 147 - DNA, 16 bp, Homo sapiens
ttattgtttctgataa

SEQ ID NO: 148 - DNA, 16 bp, Homo sapiens
tctgtatgttagcgtg

SEQ ID NO: 149 - DNA, 710 bp, Homo sapiens
ttaaataattatatttaaaagagtaggagaagaacgcaggtgccccagtagaaatcacaaccagcagccaacaccaacaacctctcatgtgaattaagccattaaaatgttgttttaaattattttattatttttcttattcttaagaaaatgttgtgggtacatagtagctgtatatatgtatggggtacatgagctgttttgatacaggcatgcaatgtgaaataagcatattgagaattaggtatccatcccctcaagcatttatcctttgggttacgaacaatccagttacattctttatgagttattttaaaatgtacaattaagttattattgactattgtcaccctattgtcccactctttatttcagccatttcagctgctcttgactgcactcaagtgagcttagcagaagaaactcacgctaacatacagawttatcagaaacaataaattaatattttaagccactaacaatgaggtcgacttattacacaacaaaagaggattaatggtacttgttaacaatgctctagaacattatgttagaatttccttaatgaatgagcatgtaaggaaactaggtaactttcatatatttgagatttaacaaatgatattaatcaacataatttaaaacacaaattctgagtctggcaaaactttgaagtattgtatataatagcgcccagaaagatacttccc

SEQ ID NO: 150 - DNA, 30 bp, Homo sapiens
taatcataagaaataattactaacatttat

SEQ ID NO: 151 - DNA, 30 bp, Homo sapiens
tgtggcttatttatgacacacattatttta

SEQ ID NO: 152 - DNA, 30 bp, Homo sapiens; identical to SEQ ID NO: 150

SEQ ID NO: 153 - DNA, 30 bp, Homo sapiens; identical to SEQ ID NO: 151

SEQ ID NO: 154 - DNA, 30 bp, Homo sapiens; identical to SEQ ID NO: 150

SEQ ID NO: 155 - DNA, 30 bp, Homo sapiens; identical to SEQ ID NO: 151

SEQ ID NO: 156 - DNA, 30 bp, Homo sapiens; identical to SEQ ID NO: 150

SEQ ID NO: 157 - DNA, 30 bp, Homo sapiens; identical to SEQ ID NO: 151

SEQ ID NO: 158 - DNA, 30 bp, Homo sapiens; identical to SEQ ID NO: 150

SEQ ID NO: 159 - DNA, 30 bp, Homo sapiens; identical to SEQ ID NO: 151

SEQ ID NO: 160 - DNA, 30 bp, Homo sapiens; identical to SEQ ID NO: 150

SEQ ID NO: 161 - DNA, 30 bp, Homo sapiens; identical to SEQ ID NO: 151

SEQ ID NO: 162 - DNA, 30 bp, Homo sapiens; identical to SEQ ID NO: 150

SEQ ID NO: 163 - DNA, 30 bp, Homo sapiens; identical to SEQ ID NO: 151

SEQ ID NO: 164 - DNA, 30 bp, Homo sapiens; identical to SEQ ID NO: 150

SEQ ID NO: 165 - DNA, 30 bp, Homo sapiens; identical to SEQ ID NO: 151

SEQ ID NO: 166 - DNA, 30 bp, Homo sapiens; identical to SEQ ID NO: 150

SEQ ID NO: 167 - DNA, 30 bp, Homo sapiens; identical to SEQ ID NO: 151

SEQ ID NO: 168 - DNA, 30 bp, Homo sapiens; identical to SEQ ID NO: 150

SEQ ID NO: 169 - DNA, 30 bp, Homo sapiens; identical to SEQ ID NO: 151

SEQ ID NO: 170 - DNA, 793 bp, Homo sapiens
gtgtcaacactgtgccatgtctcaggagctggtcttgtcaagtttcagtgggaaaccactaaacataatgagtgagatgcctgataccctataattattatagtatatacagtatgctatttatgtatcaagttgtattacatgtgtgaataatactatgttatatgtaatgtaatctcttcattagagaaagaagagttgaggaaagcactgtggtgtggtggttaagagcacaatttgaaattcccactctgttacctacttagttatctgaaagtgggcaagtattttaaactctataaacttgatttttctcttctgtaacactggggaggataataataatcataagaaataattactaacatttatrtgtggcttatttatgacacacattattttaaataccttaaatttattaacttttttaatccttccataaactctatgagggaggcaatattatccccattttagggatgaagaacctaaggtacagagaaattaagcaacatgacctgatatacacagctaacaagtggcagagccaggttacctcattattcaaactccagaggatgttcttttaaccagatcatatctatcttttaaaaaattgtttagttcatgcatggaaaacacttagtattcctggcatattaaaatgattaataaattggaactgttatcatactacattaagaatgacaccgatcatcaatttagtaacttttgttgttcaaactgtgaaataccaagtatacaaaaaagagaggaaaattaaagctttttt

SEQ ID NO: 171 - DNA, 30 bp, Homo sapiens
attgaaccatgcacttttgtagggagtggc

SEQ ID NO: 172 - DNA, 30 bp, Homo sapiens
tgtcctgtgtttgtttgaatgtctcatgta

SEQ ID NO: 173 - DNA, 30 bp, Homo sapiens; identical to SEQ ID NO: 171

SEQ ID NO: 174 - DNA, 30 bp, Homo sapiens; identical to SEQ ID NO: 172

SEQ ID NO: 175 - DNA, 30 bp, Homo sapiens; identical to SEQ ID NO: 171

SEQ ID NO: 176 - DNA, 30 bp, Homo sapiens; identical to SEQ ID NO: 172

SEQ ID NO: 177 - DNA, 30 bp, Homo sapiens; identical to SEQ ID NO: 171

SEQ ID NO: 178 - DNA, 30 bp, Homo sapiens; identical to SEQ ID NO: 172

SEQ ID NO: 179 - DNA, 30 bp, Homo sapiens; identical to SEQ ID NO: 171

SEQ ID NO: 180 - DNA, 30 bp, Homo sapiens; identical to SEQ ID NO: 172

SEQ ID NO: 181 - DNA, 30 bp, Homo sapiens
tacatgagacattcaaacaaacacaggaca

SEQ ID NO: 182 - DNA, 30 bp, Homo sapiens
gccactccctacaaaagtgcatggttcaat

SEQ ID NO: 183 - DNA, 30 bp, Homo sapiens; identical to SEQ ID NO: 171

SEQ ID NO: 184 - DNA, 30 bp, Homo sapiens; identical to SEQ ID NO: 172

SEQ ID NO: 185 - DNA, 30 bp, Homo sapiens; identical to SEQ ID NO: 171

SEQ ID NO: 186 - DNA, 30 bp, Homo sapiens; identical to SEQ ID NO: 172

SEQ ID NO: 187 - DNA, 30 bp, Homo sapiens; identical to SEQ ID NO: 171

SEQ ID NO: 188 - DNA, 30 bp, Homo sapiens; identical to SEQ ID NO: 172

SEQ ID NO: 189 - DNA, 30 bp, Homo sapiens; identical to SEQ ID NO: 171

SEQ ID NO: 190 - DNA, 30 bp, Homo sapiens; identical to SEQ ID NO: 172

SEQ ID NO: 191 - DNA, 601 bp, Homo sapiens
aatatggaaaatttaaataatattctgtgagacttatacatagttgtaaaaggaagaaaacttgttgccagtgccacttacatctgcctcttctttattgacaatcttccaaataaatttaaatagcatagttgctttgggtcctgtcgagtttggcctggaattgtaggtgattagaaaggatagggaagctttcatgtgatacgtattcattgatttattaattatttaagacctactttatattgaagtatcctacatgttataggatacatgagacattcaaacaaacacaggacaygccactccctacaaaagtgcatggttcaattgaggaaggaagtcttacagatgggacagagaaccagaaaataacataaagccaacatgtaatgtgtaggtccacctctctatctttgtagggatatagcatttcagaccaaaacctttaatttgggtctgagaattcaggaacaaatattttccttggtaaggacatttcttgcacaatttctaatagtgcaaaattggaaacaccgtgaagggcaccaacaaggcaaggttgagtaactattgttcttatcacaggacgtcacatggt

SEQ ID NO: 192 - DNA, 30 bp, Homo sapiens
cccctagcagctctccccaatctggctaac

SEQ ID NO: 193 - DNA, 30 bp, Homo sapiens
actttattgtgtttcccaacaaccatattt

SEQ ID NO: 194 - DNA, 30 bp, Homo sapiens; identical to SEQ ID NO: 192

SEQ ID NO: 195 - DNA, 30 bp, Homo sapiens; identical to SEQ ID NO: 193

SEQ ID NO: 196 - DNA, 30 bp, Homo sapiens; identical to SEQ ID NO: 192

SEQ ID NO: 197 - DNA, 30 bp, Homo sapiens; identical to SEQ ID NO: 193

SEQ ID NO: 198 - DNA, 16 bp, Homo sapiens
ccccaatctggctaac

SEQ ID NO: 199 - DNA, 16 bp, Homo sapiens
actttattgtgtttcc

SEQ ID NO: 200 - DNA, 30 bp, Homo sapiens; identical to SEQ ID NO: 192

SEQ ID NO: 201 - DNA, 30 bp, Homo sapiens; identical to SEQ ID NO: 193

SEQ ID NO: 202 - DNA, 30 bp, Homo sapiens; identical to SEQ ID NO: 192

SEQ ID NO: 203 - DNA, 30 bp, Homo sapiens; identical to SEQ ID NO: 193

SEQ ID NO: 204 - DNA, 30 bp, Homo sapiens; identical to SEQ ID NO: 192

SEQ ID NO: 205 - DNA, 30 bp, Homo sapiens; identical to SEQ ID NO: 193

SEQ ID NO: 206 - DNA, 2257 bp, Homo sapiens
ttggataagagaaattttctcccccattttgcagctaggcaaagtaagtagagcatgagttaagtagtagaaaatccagcactggcagtcaagcaccagagaatctgccccagtaatttttaatggtttccagattacaaaccactttcttcacaattgctggcaaacaccccctagcagctctccccaatctggctaacyactttattgtgtttcccaacaaccatattttaagctattgatatcctcagtccttccactcttgcctttgagattgcagtaatatcaagggtcctagaggcttccaaggtattccacctgaaactgtcctctgctttctatttatttttttattttatttatttatttattatattttttattatactttaagttctagggtacatgtgcaaaacatgcaggtatgttacatatgtatacatgtgccatgttggtgtgctgcacccattaactcgtcttttacattaggtatatctcctaatgctatccccccacccttcccccaccccacaacaggccctggtgtctgatgttccccttcttgtgtccaagtgctctcattgttcatttcccacctatgagtgagaacatgtggtgtttggttttttgtccttgtgatagtttgctgagaatgatggtttccagcttcatccatgtccctaaaaaggacatgaactcatcattttttatggctgcatagtattgcatggtgtatttgtgccacattttcttaatccagtctatcattgttggacatttggcttggttccaagtctttgctattgtgagtagtgccacaataaacatacgtgtgcatgtgtctttatagcagcatgattgatattcctttgggtatatacccagtaatgggatggctgggtcaaatggtatttctagttctagatccctgaggaattgccacactgtcttccacaatggttgaactagtttacagttccaccaacagtgtaaaagtgttcccatttctccacatcctctccagcacctgttgtttcctgactttttaatgattgccattctatctggtgtgagatgatatctcattgtggttttgatttgcatttctctgatggccagtgatgatgagcattttttcatgtgtctgttggctgcataaatgtcttcttttgagaagtgtctgttcatatcctttgcccactttttgatggggttgtttgtttttttcttgtaaatttgtttgagttctttgtagattctggatattagccttttgtcagatgagtagattgcaaaaattttctcccattctgtagttgcctgttcactctgatggtagtttcttttgctgtgcagaagctcttgagtttaattagagcccatttgtcaattttggcttttgttgccattgcttttggtgttttagacatgaagtccttgcccatgcctatgtcctgaatggtattgccctaggttttcttctagggtttttatggttttaggtctaacatttaagtctttaatccatcttgaattaatttttgtataaggtgtaaggaagggatccagtttcagctttctacatatggctagccagttttcccagcaccatttgttaaatagggaatcctttccccatttcttgtttttgtcaggtttgtcaaagatcagatagttgtagatgtgtggtattatttctgacagacaaacagagaaccaaatcatgagtgaactcccattcacaattgcttaaaagagaataaaatacctaggaatccaacttacaagggatgtgaaggacctcttcaaggagaactacaaaccagtgatcaataaaataaaaaggacacaaacaaatggaagaacattccatgctcatggataggaagaatcaatatcgtgaaaatggccatactgcataagataatttatagattcgatgccatccccatcaagctaccatgactttcttcactgaattggaaaaacctactttaaagttcatatggaaccaaaaaagagcccgcattgccaagtcaatcctaagctaaaagaacatagctggagacatcacgctacctgacttcaaactatactacaaggctacagtaaccaaaacatcatggtactggtaccaaaacagagatatagaccaatggaacataacagagccctcagaaataataccatacgtcatcctctgcattctaaatccctcactttcttccagacagattcaaagtcctttcttctttga

SEQ ID NO: 207 - DNA, 30 bp, Homo sapiens
ttttctcttctgtaacactggggaggataa

SEQ ID NO: 208 - DNA, 30 bp, Homo sapiens
aataatcataagaaataattactaacattt

SEQ ID NO: 209 - DNA, 30 bp, Homo sapiens; identical to SEQ ID NO: 207

SEQ ID NO: 210 - DNA, 30 bp, Homo sapiens; identical to SEQ ID NO: 208

SEQ ID NO: 211 - DNA, 30 bp, Homo sapiens; identical to SEQ ID NO: 207

SEQ ID NO: 212 - DNA, 30 bp, Homo sapiens; identical to SEQ ID NO: 208

SEQ ID NO: 213 - DNA, 30 bp, Homo sapiens; identical to SEQ ID NO: 207

SEQ ID NO: 214 - DNA, 30 bp, Homo sapiens; identical to SEQ ID NO: 208

SEQ ID NO: 215 - DNA, 30 bp, Homo sapiens; identical to SEQ ID NO: 207

SEQ ID NO: 216 - DNA, 30 bp, Homo sapiens; identical to SEQ ID NO: 208

SEQ ID NO: 217 - DNA, 30 bp, Homo sapiens; identical to SEQ ID NO: 207

SEQ ID NO: 218 - DNA, 30 bp, Homo sapiens; identical to SEQ ID NO: 208

SEQ ID NO: 219 - DNA, 30 bp, Homo sapiens; identical to SEQ ID NO: 207

SEQ ID NO: 220 - DNA, 30 bp, Homo sapiens; identical to SEQ ID NO: 208

SEQ ID NO: 221 - DNA, 793 bp, Homo sapiens
ataagcatggtgtcaacactgtgccatgtctcaggagctggtcttgtcaagtttcagtgggaaaccactaaacataatgagtgagatgcctgataccctataattattatagtatatacagtatgctatttatgtatcaagttgtattacatgtgtgaataatactatgttatatgtaatgtaatctcttcattagagaaagaagagttgaggaaagcactgtggtgtggtggttaagagcacaatttgaaattcccactctgttacctacttagttatctgaaagtgggcaagtattttaaactctataaacttgatttttctcttctgtaacactggggaggataayaataatcataagaaataattactaacatttatgtgtggcttatttatgacacacattattttaaataccttaaatttattaacttttttaatccttccataaactctatgagggaggcaatattatccccattttagggatgaagaacctaaggtacagagaaattaagcaacatgacctgatatacacagctaacaagtggcagagccaggttacctcattattcaaactccagaggatgttcttttaaccagatcatatctatcttttaaaaaattgtttagttcatgcatggaaaacacttagtattcctggcatattaaaatgattaataaattggaactgttatcatactacattaagaatgacaccgatcatcaatttagtaacttttgttgttcaaactgtgaaataccaagtatacaaaaaagagaggaaaattaa

SEQ ID NO: 222 - DNA, 30 bp, Homo sapiens
tctggagaagagaatgaatggaaaaagcat

SEQ ID NO: 223 - DNA, 30 bp, Homo sapiens
gattttctgaaaaaaatgaaaatgttctct

SEQ ID NO: 224 - DNA, 30 bp, Homo sapiens; identical to SEQ ID NO: 222

SEQ ID NO: 225 - DNA, 30 bp, Homo sapiens; identical to SEQ ID NO: 223

SEQ ID NO: 226 - DNA, 575 bp, Homo sapiens
tccttgcacaagttacacagcatcagttctgccaaattatattgatcaaaaccagccacagggccagttcagattcacgggtatgaaacataggtgccacatctcaataggaagagtgacaataattttatggcaatcttttcctatgatgtgcaaaaagacttatataataatgtttacagccacttcataatagtcaaactattgaaaatgacccaactgtccaacaacagatgaatgaataaataaacctgcatatatttctacaattgaatgcttaatttgaattattcaatgaatatttaattgaatacaaagtatactactaatattcacaataacatgtgtgaatctcacagacattaagttgagcaaaaaaaagccaggcataaaagagtacacattggatgattccgtttttaaaaagttcaagagtaggcaaaactaaaaatcagaacagcaatttcctctggagaagagaatgaatggaaaaagcatsgattttctgaaaaaaatgaaaatgttctcttgattagctatttacattttcaaaactcattgaactgtacacttaa

SEQ ID NO: 227 - DNA, 30 bp, Homo sapiens
agtcatctatgccttaattttatagatagt

SEQ ID NO: 228 - DNA, 30 bp, Homo sapiens
gtcctccaatgtctctagatattttctttt

SEQ ID NO: 229 - DNA, 30 bp, Homo sapiens
aaaagaaaatatctagagacattggaggac

SEQ ID NO: 230 - DNA, 30 bp, Homo sapiens
actatctataaaattaaggcataaatgact

SEQ ID NO: 231 - DNA, 30 bp, Homo sapiens; identical to SEQ ID NO: 229

SEQ ID NO: 232 - DNA, 30 bp, Homo sapiens; identical to SEQ ID NO: 230

SEQ ID NO: 233 - DNA, 30 bp, Homo sapiens; identical to SEQ ID NO: 229

SEQ ID NO: 234 - DNA, 30 bp, Homo sapiens; identical to SEQ ID NO: 230

SEQ ID NO: 235 - DNA, 30 bp, Homo sapiens; identical to SEQ ID NO: 229

SEQ ID NO: 236 - DNA, 30 bp, Homo sapiens; identical to SEQ ID NO: 230

SEQ ID NO: 237 - DNA, 30 bp, Homo sapiens; identical to SEQ ID NO: 229

SEQ ID NO: 238 - DNA, 30 bp, Homo sapiens
actatctataaaattaaggcatagatgact

SEQ ID NO: 239 - DNA, 25 bp, Homo sapiens
aaaatatctagagacattggaggac

SEQ ID NO: 240 - DNA, 25 bp, Homo sapiens
actatctataaaattaaggcataga

SEQ ID NO: 241 - DNA, 601 bp, Homo sapiens
acctcaattcacatctgaacaattcttgtcaactcatagttatacagtaagcacatttggtttcagcccttacagaaaaacttttgatggctagtattttaaatttattgattaagatactttaatgaacagactcaacattgtactatccttagcatttttggtactaattgcttttatgattgatttgcttgtgctagtctatttccttcatacttgaaagaaggatttgggaaatgtgcgttaaaaatgttctgtgtgaggcagagaagtcatctatgccttaattttatagatagtkgtcctccaatgtctctagatattttcttttaaaatagctaacataaaatgtgctttgagtcttagataatttgcttgcttccttcatgactctgctatttataagatgacttaaagaaaaaagagctaaaataacttagcactccccaacacacacatatacaaacccacaatgacaatgtaagattaataaaaaggctgtcaagttattttaaaaacaaggaacaaaataactcttctctaattctagcatttatttaaacaataattttgcataacagtatgcttttaactgaataag

SEQ ID NO: 242 - DNA, 30 bp, Homo sapiens
aaaatgttctgtgtgaggcagagaagtcat

SEQ ID NO: 243 - DNA, 30 bp, Homo sapiens
tatgccttaattttatagatagttgtcctc

SEQ ID NO: 244 - DNA, 25 bp, Homo sapiens
ccactatctataaaattaaggcata

SEQ ID NO: 245 - DNA, 30 bp, Homo sapiens
atgacttctctgcctcacacagaacatttt

SEQ ID NO: 246 - DNA, 30 bp, Homo sapiens
gaggaccactatctataaaattaaggcata

SEQ ID NO: 247 - DNA, 30 bp, Homo sapiens; identical to SEQ ID NO: 245

SEQ ID NO: 248 - DNA, 30 bp, Homo sapiens; identical to SEQ ID NO: 246

SEQ ID NO: 249 - DNA, 30 bp, Homo sapiens; identical to SEQ ID NO: 245

SEQ ID NO: 250 - DNA, 30 bp, Homo sapiens; identical to SEQ ID NO: 246

SEQ ID NO: 251 - DNA, 30 bp, Homo sapiens; identical to SEQ ID NO: 245

SEQ ID NO: 252 - DNA, 30 bp, Homo sapiens; identical to SEQ ID NO: 246

SEQ ID NO: 253 - DNA, 30 bp, Homo sapiens; identical to SEQ ID NO: 245

SEQ ID NO: 254 - DNA, 30 bp, Homo sapiens; identical to SEQ ID NO: 246

SEQ ID NO: 255 - DNA, 30 bp, Homo sapiens; identical to SEQ ID NO: 245

SEQ ID NO: 256 - DNA, 30 bp, Homo sapiens; identical to SEQ ID NO: 246

SEQ ID NO: 257 - DNA, 30 bp, Homo sapiens; identical to SEQ ID NO: 245

SEQ ID NO: 258 - DNA, 25 bp, Homo sapiens
ccactatctataaaattaaggcata

SEQ ID NO: 259 - DNA, 30 bp, Homo sapiens; identical to SEQ ID NO: 245

SEQ ID NO: 260 - DNA, 601 bp, Homo sapiens
tatgcaaaattattgtttaaataaatgctagaattagagaagagttattttgttccttgtttttaaaataacttgacagcctttttattaatcttacattgtcattgtgggtttgtatatgtgtgtgttggggagtgctaagttattttagctcttttttctttaagtcatcttataaatagcagagtcatgaaggaagcaagcaaattatctaagactcaaagcacattttatgttagctattttaaaagaaaatatctagagacattggaggaccactatctataaaattaaggcataratgacttctctgcctcacacagaacatttttaacgcacatttcccaaatccttctttcaagtatgaaggaaatagactagcacaagcaaatcaatcataaaagcaattagtaccaaaaatgctaaggatagtacaatgttgagtctgttcattaaagtatcttaatcaataaatttaaaatactagccatcaaaagtttttctgtaagggctgaaaccaaatgtgcttactgtataactatgagttgacaagaattgttcagatgtgaattgaggttttactgtctaatgcccagaggtg

SEQ ID NO: 261 - DNA, 30 bp, Homo sapiens
gttcgtaacccaaaggataaatgcttgagg

SEQ ID NO: 262 - DNA, 30 bp, Homo sapiens
gatggatacctaattctcaatatgcttatt

SEQ ID NO: 263 - DNA, 30 bp, Homo sapiens
aataagcatattgagaattaggtatccatc

SEQ ID NO: 264 - DNA, 30 bp, Homo sapiens
cctcaagcatttatcctttgggttacgaac

SEQ ID NO: 265 - DNA, 30 bp, Homo sapiens; identical to SEQ ID NO: 261

SEQ ID NO: 266 - DNA, 30 bp, Homo sapiens; identical to SEQ ID NO: 262

SEQ ID NO: 267 - DNA, 30 bp, Homo sapiens; identical to SEQ ID NO: 261

SEQ ID NO: 268 - DNA, 30 bp, Homo sapiens; identical to SEQ ID NO: 262

SEQ ID NO: 269 - DNA, 30 bp, Homo sapiens; identical to SEQ ID NO: 261

SEQ ID NO: 270 - DNA, 30 bp, Homo sapiens; identical to SEQ ID NO: 262

SEQ ID NO: 271 - DNA, 869 bp, Homo sapiens
aaattatgttgattaatatcatttgttaaatctcaaatatatgaaagttacctagtttccttacatgctcattcattaaggaaattctaacataatgttctagagcattgttaacaagtaccattaatcctcttttgttgtgtaataagtcgacctcattgttagtggcttaaaatattaatttattgtttctgataattctgtatgttagcgtgagtttcttctgctaagctcacttgagtgcagtcaagagcagctgaaatggctgaaataaagagtgggacaatagggtgacaatagtcaataataacttaattgtacattttaaaataactcataaagaatgtaactggattgttcgtaacccaaaggataaatgcttgaggsgatggatacctaattctcaatatgcttatttcacattgcatgcctgtatcaaaacagctcatgtaccccatacatatatacagctactatgtacccacaacattttcttaagaataagaaaaataataaaataatttaaaacaacattttaatggcttaattcacatgagaggttgttggtgttggctgctggttgtgatttctactggggcacctgcgttcttctcctactcttttaaatataattatttaacttctcactaataagcagaaatgaggaatggcgggagagaaggagagtaggcaagggaaggggagggaagagaagaagaaggaaaaggagagaaggaagaaaatagaaaggaaaaaacaggcaagtcatagtcccttccaacaccctcctcctctcatccttgtctcctctagtcttctcttttctagttcttcccctcacctcttctcccctttcttccactcttctt

SEQ ID NO: 272 - DNA, 30 bp, Homo sapiens
attgtgggtttgtatatgtgtgtgttgggg

SEQ ID NO: 273 - DNA, 30 bp, Homo sapiens
gtgctaagttattttagctcttttttcttt

SEQ ID NO: 274 - DNA, 30 bp, Homo sapiens; identical to SEQ ID NO: 272

SEQ ID NO: 275 - DNA, 30 bp, Homo sapiens; identical to SEQ ID NO: 273

SEQ ID NO: 276 - DNA, 30 bp, Homo sapiens
aaagaaaaaagagctaaaataacttagcac

SEQ ID NO: 277 - DNA, 30 bp, Homo sapiens
ccccaacacacacatatacaaacccacaat

SEQ ID NO: 278 - DNA, 30 bp, Homo sapiens; identical to SEQ ID NO: 272

SEQ ID NO: 279 - DNA, 30 bp, Homo sapiens; identical to SEQ ID NO: 273

SEQ ID NO: 280 - DNA, 30 bp, Homo sapiens; identical to SEQ ID NO: 272

SEQ ID NO: 281 - DNA, 30 bp, Homo sapiens; identical to SEQ ID NO: 273

SEQ ID NO: 282 - DNA, 30 bp, Homo sapiens; identical to SEQ ID NO: 272

SEQ ID NO: 283 - DNA, 30 bp, Homo sapiens; identical to SEQ ID NO: 273

SEQ ID NO: 284 - DNA, 30 bp, Homo sapiens; identical to SEQ ID NO: 272

SEQ ID NO: 285 - DNA, 30 bp, Homo sapiens; identical to SEQ ID NO: 273

SEQ ID NO: 286 - DNA, 30 bp, Homo sapiens; identical to SEQ ID NO: 272

SEQ ID NO: 287 - DNA, 30 bp, Homo sapiens; identical to SEQ ID NO: 273

SEQ ID NO: 288 - DNA, 656 bp, Homo sapiens
cgtgaggaagtagtaacagatttcttgagtcatcaaaatttcttaaggatatcagtcagcatttgatataaaacaatattttgctagtttttattaactcaaaaaatattgattaagcatcagttgattaagcctgaacttcctaacaggttttacgaagttctctttgcatgacttggtcatttaattatttattttttaaaagtcaggcctaaaatatttgtggaaagcaatgggatatgaaaaatactctactcttgctgctatgatgacaactgctgctgttaattgctgttactgtcatttgcatcattcccttccatttccacgttgagaaacacagtaaagatgatgataaactgagtgtacaaaactggttaaaagatgtttttaaaaacttattcagttaaaagcatactgttatgcaaaattattgtttaaataaatgctagaattagagaagagttattttgttccttgtttttaaaataacttgacagcctttttattaatcttacattgtcattgtgggtttgtatatgtgtgtgttggggrgtgctaagttattttagctcttttttctttaagtcatcttataaatagcagagtcatgaaggaagcaagcaaattatctaagactcaaagcacattttatg

SEQ ID NO: 289 - DNA, 30 bp, Homo sapiens
ttcagatgtgaattgaggttttactgtcta

SEQ ID NO: 290 - DNA, 30 bp, Homo sapiens
tgcccagaggtgaagcatggggtgaaaatg

SEQ ID NO: 291 - DNA, 30 bp, Homo sapiens; identical to SEQ ID NO: 289

SEQ ID NO: 292 - DNA, 30 bp, Homo sapiens; identical to SEQ ID NO: 290

SEQ ID NO: 293 - DNA, 30 bp, Homo sapiens
cattttcaccccatgcttcacctctgggca

SEQ ID NO: 294 - DNA, 30 bp, Homo sapiens
tagacagtaaaacctcaattcacatctgaa

SEQ ID NO: 295 - DNA, 30 bp, Homo sapiens; identical to SEQ ID NO: 289

SEQ ID NO: 296 - DNA, 30 bp, Homo sapiens; identical to SEQ ID NO: 290

SEQ ID NO: 297 - DNA, 30 bp, Homo sapiens; identical to SEQ ID NO: 289

SEQ ID NO: 298 - DNA, 30 bp, Homo sapiens; identical to SEQ ID NO: 290

SEQ ID NO: 299 - DNA, 30 bp, Homo sapiens; identical to SEQ ID NO: 289

SEQ ID NO: 300 - DNA, 30 bp, Homo sapiens; identical to SEQ ID NO: 290

SEQ ID NO: 301 - DNA, 30 bp, Homo sapiens; identical to SEQ ID NO: 289

SEQ ID NO: 302 - DNA, 30 bp, Homo sapiens; identical to SEQ ID NO: 290

SEQ ID NO: 303 - DNA, 30 bp, Homo sapiens; identical to SEQ ID NO: 289

SEQ ID NO: 304 - DNA, 30 bp, Homo sapiens; identical to SEQ ID NO: 290

SEQ ID NO: 305 - DNA, 651 bp, Homo sapiens
catttttaacgcacatttcccaaatccttctttcaagtatgaaggaaatagactagcacaagcaaatcaatcataaaagcaattagtaccaaaaatgctaaggatagtacaatgttgagtctgttcattaaagtatcttaatcaataaatttaaaatactagccatcaaaagtttttctgtaagggctgaaaccaaatgtgcttactgtataactatgagttgacaagaattgttcagatgtgaattgaggttttactgtctamtgcccagaggtgaagcatggggtgaaaatgggaggacagtggggagggtgaagaagttgaggggaagtatctttctgggcgctattatatacaatacttcaaagttttgccagactcagaatttgtgttttaaattatgttgattaatatcatttgttaaatctcaaatatatgaaagttacctagtttccttacatgctcattcattaaggaaattctaacataatgttctagagcattgttaacaagtaccattaatcctcttttgttgtgtaataagtcgacctcattgttagtggcttaaaatattaatttattgtttctgataattctgtatgttagcgtgagtttcttctgctaagctcacttgagtgcagtcaagagcag

SEQ ID NO: 306 - DNA, 30 bp, Homo sapiens
ggcaaaactttgaagtattgtatataatag

SEQ ID NO: 307 - DNA, 30 bp, Homo sapiens
gcccagaaagatacttcccctcaacttctt

SEQ ID NO: 308 - DNA, 30 bp, Homo sapiens
aagaagttgaggggaagtatctttctgggc

SEQ ID NO: 309 - DNA, 30 bp, Homo sapiens
ctattatatacaatacttcaaagttttgcc

SEQ ID NO: 310 - DNA, 30 bp, Homo sapiens; identical to SEQ ID NO: 306

SEQ ID NO: 311 - DNA, 30 bp, Homo sapiens; identical to SEQ ID NO: 307

SEQ ID NO: 312 - DNA, 30 bp, Homo sapiens; identical to SEQ ID NO: 308

SEQ ID NO: 313 - DNA, 30 bp, Homo sapiens; identical to SEQ ID NO: 309

SEQ ID NO: 314 - DNA, 30 bp, Homo sapiens; identical to SEQ ID NO: 308

SEQ ID NO: 315 - DNA, 30 bp, Homo sapiens; identical to SEQ ID NO: 309

SEQ ID NO: 316 - DNA, 30 bp, Homo sapiens; identical to SEQ ID NO: 308

SEQ ID NO: 317 - DNA, 30 bp, Homo sapiens; identical to SEQ ID NO: 309

SEQ ID NO: 318 - DNA, 30 bp, Homo sapiens; identical to SEQ ID NO: 308

SEQ ID NO: 319 - DNA, 30 bp, Homo sapiens; identical to SEQ ID NO: 309

SEQ ID NO: 320 - DNA, 25 bp, Homo sapiens
gttgaggggaagtatctttctgggc

SEQ ID NO: 321 - DNA, 25 bp, Homo sapiens
ctattatatacaatacttcaaagtt

SEQ ID NO: 322 - DNA, 651 bp, Homo sapiens
catttttaacgcacatttcccaaatccttctttcaagtatgaaggaaatagactagcacaagcaaatcaatcataaaagcaattagtaccaaaaatgctaaggatagtacaatgttgagtctgttcattaaagtatcttaatcaataaatttaaaatactagccatcaaaagtttttctgtaagggctgaaaccaaatgtgcttactgtataactatgagttgacaagaattgttcagatgtgaattgaggttttactgtctactgcccagaggtgaagcatggggtgaaaatgggaggacagtggggagggtgaagaagttgaggggaagtatctttctgggcrctattatatacaatacttcaaagttttgccagactcagaatttgtgttttaaattatgttgattaatatcatttgttaaatctcaaatatatgaaagttacctagtttccttacatgctcattcattaaggaaattctaacataatgttctagagcattgttaacaagtaccattaatcctcttttgttgtgtaataagtcgacctcattgttagtggcttaaaatattaatttattgtttctgataattctgtatgttagcgtgagtttcttctgctaagctcacttgagtgcagtcaagagcag

SEQ ID NO: 323 - DNA, 30 bp, Homo sapiens
acaataccgaagttaaaatcattagctaca

SEQ ID NO: 324 - DNA, 30 bp, Homo sapiens
attacagcatacaggctaaatgtctacata

SEQ ID NO: 325 - DNA, 30 bp, Homo sapiens
tatgtagacatttagcctgtatgctgtaat

SEQ ID NO: 326 - DNA, 30 bp, Homo sapiens
tgtagctaatgattttaacttcggtattgt

SEQ ID NO: 327 - DNA, 16 bp, Homo sapiens
aaaatcattagctaca

SEQ ID NO: 328 - DNA, 16 bp, Homo sapiens
attacagcatacaggc

SEQ ID NO: 329 - DNA, 30 bp, Homo sapiens; identical to SEQ ID NO: 323

SEQ ID NO: 330 - DNA, 30 bp, Homo sapiens; identical to SEQ ID NO: 324

SEQ ID NO: 331 - DNA, 30 bp, Homo sapiens; identical to SEQ ID NO: 323

SEQ ID NO: 332 - DNA, 30 bp, Homo sapiens; identical to SEQ ID NO: 324

SEQ ID NO: 333 - DNA, 30 bp, Homo sapiens; identical to SEQ ID NO: 323

SEQ ID NO: 334 - DNA, 30 bp, Homo sapiens; identical to SEQ ID NO: 324

SEQ ID NO: 335 - DNA, 707 bp, Homo sapiens
tgacagggagaaaaatcattccaattaaatatgctctactgctaaactctaatttaaagaaaacaataaatgaggtggagaaaaataagaaatacattcatcaactgaaactaaggctagtgaggcagaaataattttatacatgtttattgaaagccaactgggaagacagacctgggaagacacatccacaaagttgggggtgttctagagtctttcacaagttacaagggctttatataatagaaaagcttaaaataaggcagaaaaactcctcataccagagttgtcttctttttcattggagggtacaataccgaagttaaaatcattagctacarattacagcatacaggctaaatgtctacatacaaaacaatcaataaaactttttgtttcagctaaacttagaaataaatcattgtcctatttagtgtcagtaggttctacagtaattagtacatcaatcatctgaggaactcgtaataaaatactttactcagagatagaatgtcacccatcaagtcacgaaacctttccagggcgagttaatttggaagtctgcttacttctaaagtaaactgtcaaatgtggtctgtaggttatcgcatttaaaaacaggacctgagaacttgagtagacatagtagataactattaaacaatagggaaaattggcttgaaaacaaaaacatgatttaaaaaatt

SEQ ID NO: 336 - DNA, 30 bp, Homo sapiens
ttaacgcacatttcccaaatccttctttca

SEQ ID NO: 337 - DNA, 30 bp, Homo sapiens
gtatgaaggaaatagactagcacaagcaaa

SEQ ID NO: 338 - DNA, 401 bp, Homo sapiens
ctttaagtcatcttataaatagcagagtcatgaaggaagcaagcaaattatctaagactcaaagcacattttatgttagctattttaaaagaaaatatctagagacattggaggaccactatctataaaattaaggcataaatgacttctctgcctcacacagaacatttttaacgcacatttcccaaatccttctttcargtatgaaggaaatagactagcacaagcaaatcaatcataaaagcaattagtaccaaaaatgctaaggatagtacaatgttgagtctgttcattaaagtatcttaatcaataaatttaaaatactagccatcaaaagtttttctgtaagggctgaaaccaaatgtgcttactgtataactatgagttgacaagaattgttc

SEQ ID NO: 339 - DNA, 30 bp, Homo sapiens
attttcaatagtttgactattatgaagtgg

SEQ ID NO: 340 - DNA, 30 bp, Homo sapiens
tgtaaacattattatataagtctttttgca

SEQ ID NO: 341 - DNA, 2558 bp, Homo sapiens
ggttccttcagccacccatgaatacttctcttatccgaatttaagtcattcttctgccacaaactttctgtatgatctcaaacaatttactatttatttaaatgaaaatactaatatcagctttcttctccataaaactattcagaagaacaaaaaggatagctgtgaaaacttttaaaaatattttttcttcttttatgttttacattgatgtaaaattttcacagtgagatgtacatatcttaagtgtacagttcaatgagttttgaaaatgtaaatagctaatcaagagaacattttcattttttttcagaaaatccatgctttttccattcattctcttctccagaggaaattgctgttctgatttttagttttgcctactcttgaactttttaaaaacggaatcatccaatgtgtactcttttatgcctggcttttttttgctcaacttaatgtctgtgagattcacacatgttattgtgaatattagtagtatactttgtattcaattaaatattcattgaataattcaaattaagcattcaattgtagaaatatatgcaggtttatttattcattcatctgttgttggacagttgggtcattttcaatagtttgactattatgaagtggytgtaaacattattatataagtctttttgcacatcataggaaaagattgccataaaattattgtcactcttcctattgagatgtggcacctatgtttcatacccgtgaatctgaactggccctgtggctggttttgatcaatataatttggcagaactgatgctgtgtaacttgtgcaaggatatctacagcttccaccttggtcttttggaatgattcttcttggaacccaacccctgtgctttgagaagcctgagctatgtgtagaggccgtatggaagagaaccaaggcactccagctgatgggcctcactgggctcccagctgagagccagcaccaacctgcaaccgtgtaagcaatcgtcttgtactttccaacctagtcaaatttcagatgtctgcaactccagccacatggagcaaaagattcacagagatgagtccagtcccctatcaacccagataatcctgagacaataaatagctattgttttaacccactaagttttgagataattttttacacagaaacaatcaaattatttattttcacttcttcaggataagtattaggaatagaactgctggaccatataatacgtgtgtattaatctttatagaaaattaccaaattattaccagaggtagttttaccttttcatactcctactagcaaagtaagagtgccctggtttctccatatcctcgtcataactgtttttaaatttttagctactggcagttattcatgctatcccattgggtttttaatttttatttcatgattactaataatgttgaaaacttttccttgtgtttaattatcatttgtaaattttcctttgtgaaatctatttacaaatcttttgtctattttgttgtaaattgagtttttgccattttatatttgtcggagttaattgtatattgtagatacaagttctttgattgatagataaatgtattgtgctttttttttttttttttttttttttttgagacggagtctagctctgtcgcccaggctggagtgcagtggcgcagtctcggctcacttcaagctccgcctcccgggttcacaccattctcctgcctcagcctcctgattagctgggactacaggcctccgccaccacgcccagctaattgtttgtatttttagtagagacagggtttcaccgtgttagccaggatggtctccatctcctgacctcgtgatccgcccgcctcagccgcccaaagtgctgggattacaggcgtgagccactgcccccagcccttgtgattttttagtctatagcttgactttttattttcttaagaatgtctttgaggaggagaaagtttgaattctgataaagtcaaacttaccaatttttaaatgattaatcctattttatgtttgtcctctctgagaaattattgtctacctccatggttgaaagatattcttttgtaagttttcaaaagtttttgtttaccttttatattcatatatataagccatattgaattaatttttataaatgatccaaaatagtaattgagatacattgtttcactttttaatataactgtcaaattattataacaagatttttgaaaacttttctcattaaatttactcagaaccattaccagaattcaatgatggcatacatgtgcatctatttctagactttatttgatttcatggtctatgtgtctcttcttacaccaaatgtccttataaatctagctttatagtaagccttgaaatcagatagtgcatcagctaaaggctgtagcattggtcaatttgaatgcttcctttctatctttagcttttaaaatggcttccttcctttagcatcatatcatattgaatggagcatctgggatctccattttgaagcatatgtctgtaggcagccatagcccct

SEQ ID NO: 342 - DNA, 30 bp, Homo sapiens
catattttaagctattgatatcctcagtcc

SEQ ID NO: 343 - DNA, 30 bp, Homo sapiens
tccactcttgcctttgagattgcagtaata

SEQ ID NO: 344 - DNA, 30 bp, Homo sapiens; identical to SEQ ID NO: 342

SEQ ID NO: 345 - DNA, 30 bp, Homo sapiens; identical to SEQ ID NO: 343

SEQ ID NO: 346 - DNA, 30 bp, Homo sapiens; identical to SEQ ID NO: 342

SEQ ID NO: 347 - DNA, 30 bp, Homo sapiens; identical to SEQ ID NO: 343

SEQ ID NO: 348 - DNA, 2257 bp, Homo sapiens
atgagttaagtagtagaaaatccagcactggcagtcaagcaccagagaatctgccccagtaatttttaatggtttccagattacaaaccactttcttcacaattgctggcaaacaccccctagcagctctccccaatctggctaaccactttattgtgtttcccaacaaccatattttaagctattgatatcctcagtccwtccactcttgcctttgagattgcagtaatatcaagggtcctagaggcttccaaggtattccacctgaaactgtcctctgctttctatttatttttttattttatttatttatttattatattttttattatactttaagttctagggtacatgtgcaaaacatgcaggtatgttacatatgtatacatgtgccatgttggtgtgctgcacccattaactcgtcttttacattaggtatatctcctaatgctatccccccacccttcccccaccccacaacaggccctggtgtctgatgttccccttcttgtgtccaagtgctctcattgttcatttcccacctatgagtgagaacatgtggtgtttggttttttgtccttgtgatagtttgctgagaatgatggtttccagcttcatccatgtccctaaaaaggacatgaactcatcattttttatggctgcatagtattgcatggtgtatttgtgccacattttcttaatccagtctatcattgttggacatttggcttggttccaagtctttgctattgtgagtagtgccacaataaacatacgtgtgcatgtgtctttatagcagcatgattgatattcctttgggtatatacccagtaatgggatggctgggtcaaatggtatttctagttctagatccctgaggaattgccacactgtcttccacaatggttgaactagtttacagttccaccaacagtgtaaaagtgttcccatttctccacatcctctccagcacctgttgtttcctgactttttaatgattgccattctatctggtgtgagatgatatctcattgtggttttgatttgcatttctctgatggccagtgatgatgagcattttttcatgtgtctgttggctgcataaatgtcttcttttgagaagtgtctgttcatatcctttgcccactttttgatggggttgtttgtttttttcttgtaaatttgtttgagttctttgtagattctggatattagccttttgtcagatgagtagattgcaaaaattttctcccattctgtagttgcctgttcactctgatggtagtttcttttgctgtgcagaagctcttgagtttaattagagcccatttgtcaattttggcttttgttgccattgcttttggtgttttagacatgaagtccttgcccatgcctatgtcctgaatggtattgccctaggttttcttctagggtttttatggttttaggtctaacatttaagtctttaatccatcttgaattaatttttgtataaggtgtaaggaagggatccagtttcagctttctacatatggctagccagttttcccagcaccatttgttaaatagggaatcctttccccatttcttgtttttgtcaggtttgtcaaagatcagatagttgtagatgtgtggtattatttctgacagacaaacagagaaccaaatcatgagtgaactcccattcacaattgcttaaaagagaataaaatacctaggaatccaacttacaagggatgtgaaggacctcttcaaggagaactacaaaccagtgatcaataaaataaaaaggacacaaacaaatggaagaacattccatgctcatggataggaagaatcaatatcgtgaaaatggccatactgcataagataatttatagattcgatgccatccccatcaagctaccatgactttcttcactgaattggaaaaacctactttaaagttcatatggaaccaaaaaagagcccgcattgccaagtcaatcctaagctaaaagaacatagctggagacatcacgctacctgacttcaaactatactacaaggctacagtaaccaaaacatcatggtactggtaccaaaacagagatatagaccaatggaacataacagagccctcagaaataataccatacgtcatcctctgcattctaaatccctcactttcttccagacagattcaaagtcctttcttctttgaaaaggcttattctgacaaagtcatttgtagggtattccgtccacctttgctctt

SEQ ID NO: 349 - DNA, 30 bp, Homo sapiens
tgctatttaaatttatttggaagattgtca

SEQ ID NO: 350 - DNA, 30 bp, Homo sapiens
taaagaagaggcagatgtaagtggcactgg

SEQ ID NO: 351 - DNA, 30 bp, Homo sapiens; identical to SEQ ID NO: 349

SEQ ID NO: 352 - DNA, 30 bp, Homo sapiens; identical to SEQ ID NO: 350

SEQ ID NO: 353 - DNA, 30 bp, Homo sapiens; identical to SEQ ID NO: 349

SEQ ID NO: 354 - DNA, 30 bp, Homo sapiens; identical to SEQ ID NO: 350

SEQ ID NO: 355 - DNA, 25 bp, Homo sapiens
tttaaatttatttggaagattgtca

SEQ ID NO: 356 - DNA, 25 bp, Homo sapiens
taaagaagaggcagatgtaagtggc

SEQ ID NO: 357 - DNA, 401 bp, Homo sapiens
tcctgtgtttgtttgaatgtctcatgtatcctataacatgtaggatacttcaatataaagtaggtcttaaataattaataaatcaatgaatacgtatcacatgaaagcttccctatcctttctaatcacctacaattccaggccaaactcgacaggacccaaagcaactatgctatttaaatttatttggaagattgtcartaaagaagaggcagatgtaagtggcactggcaacaagttttcttccttttacaactatgtataagtctcacagaatattatttaaattttccatattcttttcttacttttcatatccttaaattttagaagaaatgcttaaaatactaaatgttcaaaattactctctatcaaaaagatgaatatagtaattttttaaa

(19) United States
(12) Patent Application Publication
(10) Pub. No.: US 2008/0045582 A1
(43) Pub. Date: Feb. 21, 2008

(54) ENA-78 Gene Polymorphisms and Protein Concentrations as Diagnostic and Prognostic Tools

(76) Inventors: Issam Zineh, Alachua, FL (US); Hooman Allayee, Los Angeles, CA (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/748,964

(22) Filed: May 15, 2007

Related U.S. Application Data
(60) Provisional application No. 60/800,329, filed on May 15, 2006.

(51) Int. Cl. A61K 31/40; A61P 29/00; C12Q 1/68

(52) U.S. Cl. 514/423; 435/6

(57) ABSTRACT

The invention concerns polymorphisms in the CXCL5 gene and the concentration of epithelial neutrophil activating peptide (ENA-78) in patients. The invention also pertains to methods and systems for detecting such polymorphisms. The invention further relates to the use of such methods and systems in the diagnosis, prognosis, and treatment selection for inflammatory disorders associated with elevated ENA-78 concentrations.

### CROSS-REFERENCE TO RELATED APPLICATION

This application claims the benefit of U.S. Application Ser. No. 60/800,329, filed May 15, 2006, which is hereby incorporated by reference herein in its entirety, including any figures, tables and sequences.

### BACKGROUND OF THE INVENTION

The environment contains a variety of infectious microbial agents, such as viruses, bacteria, fungi and parasites, any one of which can cause pathological damage to the host organism. Consequently, most organisms, such as mammals, i.e. humans, have developed an immune system. The immune system is divided into two functional divisions, the innate immune system and the adaptive immune system.

The innate and adaptive immune system consists of a variety of molecules and cells distributed throughout the body. Among the most important cells are leukocytes. (also known as white blood cells) Leukocytes are categorized as phagocytes, including polymorphonuclear neutrophils (PMNs), monocytes and macrophages, and lymphocytes, which mediate both innate and adaptive immunity.

Inflammation is a localized protective response in vascularized tissue induced by microbial invasion or cell and tissue injury, such as an invasion by an infectious microbial agent and includes three broad actions. First, the blood supply is increased to the area. Second, capillary permeability is increased, thereby permitting larger molecules to reach the site of infection. Third, leukocytes, particularly PMNs, migrate out of the capillaries and into the surrounding tissue. Once in the tissue, the PMNs migrate to the site of infection or injury by chemotaxis. These events manifest themselves as inflammation. Examples of conditions which cause these reactions to occur include clamping or tourniquet vessel-induced ischemia reperfusion injury, chronic inflammatory conditions such as asthma, rheumatoid arthritis, and inflammatory bowel disease, as well as autoimmune diseases. Inflammation may also be systemic in nature.

Once at the site of infection, PMNs perform phagocytic and degradative functions to combat the infectious agent or perpetuate the inflammatory response. As part of the inflammatory response, PMNs generate superoxide anions, reactive oxygen species (ROS) and adhere to epithelial cells of mucosal surfaces or vascular endothelial cells of the blood vessels. As a consequence, the host can experience undesirable side effects during the inflammatory process such as, pain, swelling about the site, and nausea.

Chemokines are a superfamily of forty or more small (approximately about 4 to about 14 kDa) inducible and secreted pro-inflammatory cytokines that act primarily as chemoattractants and activators of specific leukocyte cell subtypes. Together, chemokines target the entire spectrum of leukocyte subtypes; individually each targets only part of the spectrum.

There are four major groups of chemokines, three of which include four conserved cysteines. The groups are defined by the arrangement of the first two cysteines. If the first two cysteines are separated by a single amino acid they are members of the CXC family (also called α); if the cysteines are adjacent, they are classified in the CC family (also called β). If they are separated by three amino acids CX₃C, they are members of the third group. The fourth group of chemokines contains two cysteines, corresponding to the first and third cysteines in the other groups. Structural analysis demonstrates that most chemokines function as monomers and that the two regions necessary for receptor binding reside within the first 35 amino acids of the flexible N-terminus (Clark-Lewis et al. (1995) J Leukoc Biol 57, 703-11; Beall et al. (1996) Biochem J 313, 633-40; and Steitz et al. (1998) FEBS Lett 430, 158-64).

Epithelial neutrophil activating peptide-78 (ENA-78) is a CXC (α) chemokine that is a neutrophil attractor and activator. It was initially discovered from the conditioned medium of human pulmonary epithelial cells that were stimulated with TNF-α or IL-1β. ENA-78 is an 8.3 kDa protein with 78 amino acids containing 4 cysteines positioned identically to those of IL-8. ENA-78 shares several properties of neutrophil activation with NAP-2 and IL-8. ENA-78 induces chemotactic activity in neutrophils, as well as release of elastase from cytochalesine-B-treated neutrophils and the induction of cytosolic calcium release. Neutrophils migrate in response to ENA-78 into inflamed areas of patients (such as inflamed joints of patients with rheumatoid arthritis).

Neutrophil activation is often a component of detrimental inflammatory processes underlying many diseases. In fact, studies have shown that maladaptive immunological processes involving neutrophils contribute to pathogenesis of atherosclerosis, diabetes, pulmonary disease, cancer, and other diseases (Aras R et al., “The proinflammatory and hypercoagulable state of diabetes mellitus,” Rev Cardiovasc Med, 6:84-97 (2005); Bisset L R and Schmid-Grendelmeier P, “Chemokines and their receptors in the pathogenesis of allergic asthma: progress and perspective,” Curr Opin Pulm Med, 11:35-42 (2005); Hansson G K, “Regulation of immune mechanisms in atherosclerosis,” Ann NY Acad Sci, 947:157-65 (2001); Pettersen C A, Adler K B, “Airways inflammation and COPD: epithelialneutrophil interactions,” Chest, 121:142S-50S (2002); Robinson S C, Coussens L M, “Soluble mediators of inflammation during tumor development,” Adv Cancer Res, 93:159-87 (2005); Stockley R A, “Neutrophils and the pathogenesis of COPD,” Chest, 121:151S-5S (2002); Wislez M et al., “Upregulation of bronchioloalveolar carcinoma-derived C-X-C chemokines by tumor infiltrating inflammatory cells,” Inflamm Res, 53:4-12 (2004); Koch A E et al., “Regulation of angiogenesis by the C-X-C chemokines interleukin-8 and epithelial neutrophil activating peptide 78 in the rheumatoid joint,” Arthritis Rheum, 44:31-40 (2001); and Ross R., “Atherosclerosis is an inflammatory disease,” N Engl J Med, 340:115-26 (1999)).

It has been hypothesized that ENA-78 is involved in pathological inflammatory processes. Data exist linking elevated ENA-78 concentrations with a myriad of inflammatory conditions (Walz A, et al., “Regulation and function of the CXC chemokine ENA-78 in monocytes and its role in disease,” J Leukoc Biol, 62:604-11 (1997)). For example, this CXC (α) chemokine has been implicated in pulmonary disease, lung cancer, arthritis, and other pathological states (Nakayama S et al., “Comparison of BALF concentrations of ENA-78 and IP10 in patients with idiopathic pulmonary fibrosis and nonspecific interstitial pneumonia,” Respir Med, 99:1145-51 (2005); Wislez M et al., “Upregulation of bronchioloalveolar carcinoma-derived C-X-C chemokines by tumor infiltrating inflammatory cells,” Inflamm Res, 53:4-12 (2004); Koch A E et al., “Regulation of angiogenesis by the C-X-C chemokines interleukin-8 and epithelial neutrophil activating peptide 78 in the rheumatoid joint,” Arthritis Rheum, 44:31-40 (2001); Walz A et al., “Neutrophil-activating peptide ENA-78,” Adv Exp Med Biol, 351:129-37 (1993); and Walz A et al., “Regulation and function of the CXC chemokine ENA-78 in monocytes and its role in disease,” J Leukoc Biol, 62:604-11 (1997)).

ENA-78 is encoded by the CXCL5 gene that, as shown in recent studies, could include polymorphisms in nature (Chang M S et al., “Cloning and characterization of the human neutrophil-activating peptide (ENA-78) gene,” J Biol Chem, 269:25277-82 (1994)). Specifically, two single nucleotide polymorphisms (SNPs), previously described as the promoter −156G/C (rs352046) and exonic 398G/A (rs425535) SNPs, have been shown to occur with relatively high allele frequencies in largely European and United States populations (Amoli M M et al., “Two polymorphisms in the epithelial cell-derived neutrophil-activating peptide (ENA-78) gene,” Dis Markers, 21:75-7 (2005); Zineh I et al., “Development and cross-validation of sequencing-based assays for genotyping common polymorphism of the CXCL5 gene,” Clin Chim Acta (Epub 2006, Mar. 27). However, prior to the subject invention, no investigations have assessed whether CXCL5 polymorphisms affect ENA-78 protein concentrations.

Given the importance of ENA-78 concentrations in immune processes, in particular the body's inflammatory response, there is a need in the art for methods for identifying polymorphisms in genes related to neutrophil activation and to correlate the identity of these polymorphisms with ENA-78 concentrations as well as inflammatory conditions associated with elevated ENA-78 concentrations. Furthermore, there is a need for methods for using CXCL5 genetic information and ENA-78 protein concentrations to prognosticate disease risk and severity, as well as likelihood of drug responses.

### BRIEF SUMMARY OF THE INVENTION

The present invention addresses the above-described needs and more by providing techniques for detecting CXCL5 genetic polymorphisms and for correlating the identity of these polymorphisms with ENA-78 concentrations and ENA-78-associated inflammatory conditions. Accordingly, the subject invention is useful for the diagnosis, prognosis, and treatment of such inflammatory conditions as, but not limited to, cardiovascular diseases, obesity, and diabetes.

In one embodiment, the present invention provides methods and compositions that allow the predictive assessment of an individual's predisposition to developing an ENA-78-associated inflammatory condition. The methods are carried out by determining the identity of one or more polymorphisms in the CXCL5 gene (AF349466), the gene encoding ENA-78. The CXCL5 gene for use in this invention includes promoter sequences, intron sequences, protein-coding sequences, and 5′- and 3′-untranslated sequences.

The invention also provides methods for correlating the identity of such polymorphisms with a genetic predisposition for a disease, particularly an inflammatory disease, and more particularly diabetes; respiratory disease such as asthma; cardiovascular-related conditions such as, but not limited to, hypertension, congestive heart failure, stroke, myocardial infarction and obstructive peripheral vascular disease, cancer, migraine, arthritis, and lupus.

To the extent that ENA-78 is involved in early neutrophil-mediated adverse processes contributing to many diseases, and CXCL5 genetic variability contributes to variable risk profiles (Zineh I et al., “CXCL5 gene polymorphisms are related to systemic concentrations and leukocyte production of epithelial neutrophil-activating peptide (ENA-78),” Cytokine 2006 Mar. 7; 33(5):258-263), the subject invention provides materials and methods for identifying CXCL5 polymorphisms that are associated with differences in: (1) circulating plasma concentrations of ENA-78 among adults, and (2) production of ENA-78 from leukocytes, where such associations are used to develop tests for patient risk stratification as well as to guide therapeutic approaches in inflammatory diseases, in particular cardiovascular disease-related conditions. In a related embodiment, CXCL5 genotypes are associated with C-reactive protein concentrations, systolic and diastolic blood pressure, and apolipoprotein B/apolipoprotein A ratio—all risk factors for cardiovascular disease and predictors of subsequent cardiovascular events, where the association is used to develop tests for patient risk stratification as well as to guide therapeutic approaches in cardiovascular disease events.

In one embodiment, the invention encompasses diagnostic methods for determining predisposition to inflammatory disease in a patient comprising: identifying the allelic pattern of CXCL5 genes in the patient; comparing the CXCL5 allelic pattern of the patient with the corresponding allelic patterns of healthy patients and those with one or more clinical indicators of present or future inflammatory disease; and determining which of said corresponding allelic patterns is most similar to the allelic pattern of the patient. If the CXCL5 allelic pattern of the patient is most similar to the corresponding allelic pattern of patients with clinical indicators of an inflammatory disease, the patient is categorized as having a predisposition to develop an inflammatory disease.

In one embodiment, two single nucleotide polymorphisms (SNPs), the promoter −356G→C (rs352046) and exonic 398G→A (rs4255350), are detected and associated with significant ENA-78 plasma concentrations in a patient and leukocyte production of ENA-78. According to the subject invention, these two SNPs are linked with one another such that a genetic test for one SNP provides the corresponding genotype at the other SNP locus in the majority of individuals tested to date.

The present invention also encompasses establishing a statistically significant correlation between CXCL5 allelic patterns and the presence or absence of one or more clinical indicators of present or future inflammatory disease. In one embodiment, the subject invention provides materials and methods that enable the skilled artisan to associate CXCL5 polymorphisms with differences in plasma ENA-78 concentrations (or leukocyte production of ENA-78 from cultured leukocytes) in patients. According to the subject invention, patients are genotyped for CXCL5 polymorphisms, where variant alleles at both loci are highly linked (D′=1, r²=0.94), where such polymorphisms are associated with increased ENA-78 plasma concentration and/or increased leukocyte production of ENA-78.

The present invention provides materials and methods useful in the treatment of a patient, such as a human patient, who has been diagnosed with an inflammatory disease in accordance with the invention. The present invention also provides materials and methods to inhibit development of an inflammatory disease associated with higher than normal levels of systemically circulating ENA-78 levels. In one embodiment, the present invention provides a method of treating elevated ENA-78 levels by administering an effective amount of a pharmaceutical composition comprising an inhibitor of ENA-78. In a related embodiment of the invention, ENA-78 production from endothelial cells is reduced upon treatment with atorvastatin.

To push application of genotype-guided diagnostics and therapy forward, medium- to high-throughput assays must be available to serve as a rapid aid to research and practice. As such, assays were developed and cross-validated using two DNA-based methods for genotype determination of the described CXCL5 −156G→C and 398G→A SNPs. Specifically, assays were developed for the non-gel, luciferase-based Pyrosequencing™ platform (Biotage, Uppsala, Sweden) and compared with commercially available assays for the fluorescence-based TaqMan® platform (Applied Biosystems, Foster City, USA). Furthermore, because of the paucity of data regarding population distribution of CXCL5 variant alleles, allele and genotype frequencies in a U.S. population were compared with those of a European population.

In accordance with the subject invention, such polymorphisms identified in the CXCL5 gene enable diagnosis of inflammatory conditions associated with elevated ENA-78 concentrations and/or identification of patient predisposition to developing such inflammatory conditions. Moreover, the subject invention advantageously provides efficacious pharmacogenetic materials and methods for the treatment and/or prevention of such inflammatory conditions. In one embodiment, the present invention provides inhibitors of neutrophil activating peptides (such as atorvastatin), in particular inhibitors of ENA-78, to treat disorders characterized by the presence of a high concentration of ENA-78.

Additional advantages of this invention will become readily apparent from the ensuing description.

### BRIEF DESCRIPTION OF THE FIGURES

FIG. 1 is a graphical illustration of plasma ENA-78 concentrations by CXCL5 −156G→C genotypes.

FIG. 2 is a graphical illustration of the CXCL5 −156G→C genotype association with ENA-78 concentrations from leukocytes.

FIGS. 3A-3C are graphical illustrations of visual genotype outputs for CXCL5 398G→A polymorphism by theoretical histogram and actual pyrograms using Pyrosequencing®.

FIG. 3D is a graphical illustration of visual genotype outputs for CXCL5 398G→A polymorphism by clustering of genotypes using TaqMan®.

FIG. 4 is a graphical illustration of the relationship between systemic C-reactive protein concentrations and CXCL5 genotypes.

FIGS. 5A1 and 5A2 are graphical illustrations of the relationship between the CXCL5 −156G→C polymorphism and systolic and diastolic blood pressure.

FIGS. 5B1 and 5B2 are graphical illustrations of the relationship between the CXCL5 −398G→A polymorphism and systolic and diastolic blood pressure.

FIG. 6 is a graphical illustration of the relationship between CXCL5 398G→A genotype and clustering of cardiovascular risk factors.

FIG. 7 is a graphical illustration of the differences in allele specific cDNA transcripts from 398G/A heterozygous patients.

FIG. 8 is a graphical illustration of the ability of fenofibrate to lower endothelial ENA-78 production in a dose-dependent fashion.

FIG. 9 is a graphical illustration allele-specific expression of CXCL5 in 398A variant carriers.

FIGS. 10A-10C are graphical illustrations of the effect of atorvastatin and mevalonateon endothelium-derived chemokines.

FIG. 11A-11B are graphical illustrations of the effect of atorvastatin and mevalonateon endothelium-derived cytokines.

FIG. 12A-12B are graphical illustrations of the effect of atorvastatin and mevalonateon endothelium-derived angiogenic factors.

FIG. 13 is a graphical illustration demonstrating cell viability of HUVECs cultured for 24 hours with atorvastatin (AT).

FIG. 14 is a graphical illustration demonstrating atorvastatin (AT) ability to attenuate interleukin-1beta (IL-1beta) induced epithelial neutrophil activating peptide-78 (ENA-78) production in a dose dependent fashion.

FIG. 15 is a graphical illustration demonstration atorvastatin (AT) ability to attenuate epithelial neutrophil activating peptide-78 (ENA-78) production by interleukin-1beta (IL-1beta) over time.

FIG. 16 is a graphical illustration of Mevalonate (MEV) and its metabolites's effect on atorvastatin (AT) in conjunction with interleukin-1beta (IL-1beta); farnesyl pyrophosphate (FPP), geranylgeranyl pyrophosphate (GGPP) as compared to IL-1beta stimulation alone.

FIGS. 17A and 17B are illustrations regarding CXCL5 gene expression, where FIG. 17A is a depiction of gel electrophoresis of PCR product of CXCL5 and GAPDH; and FIG. 17B is a graphical illustration of the Log₁₀ Relative Quantification of CXCL5 Modulated by atorvastatin (AT) and interleukin-1beta (IL-1beta).

### BRIEF DESCRIPTION OF THE APPENDICES

Also attached and made a part of this application is Appendix A, which provides detailed listings of various single nucleotide polymorphisms (SNPs). This Appendix is incorporated by reference in this application in its entirety to the same extent as if fully set forth herein.

### BRIEF DESCRIPTION OF THE SEQUENCES

SEQ ID NO:1 is the sequence for a biotinylated forward primer used to amplify a 121 bp amplicon containing the −156G→C promoter polymorphism.

SEQ ID NO:2 is the sequence for a non-biotinylated reverse primer used to amplify a 121 bp amplicon containing the −156G→C promoter polymorphism.

SEQ ID NO:3 is the sequence for a non-biotinylated forward primer used to amplify a 73 bp amplicon containing the 398G→A polymorphism in exon 2.

SEQ ID NO:4 is the sequence for a biotinylated reverse primer used to amplify a 73 bp amplicon containing the 398G→A polymorphism in exon 2.

SEQ ID NO:5 is the sequence for a reverse primer used to assay the −156G→C polymorphism.

SEQ ID NO:6 is the sequence for a forward primer used to assay the 398G→A polymorphism in exon 2.

SEQ ID NO:7 is the sequence for an exonic primer for CXCL5.

SEQ ID NO:8 is the sequence for another exonic primer for CXCL5.

SEQ ID NO:9 is the sequence for an exonic primer for GAPDH.

SEQ ID NO:10 is the sequence for an exonic primer for GAPDH.

### DETAILED DESCRIPTION OF THE INVENTION

As described above, epithelial cell-derived neutrophil-activating peptide (ENA-78) is involved in pathological inflammatory processes and potentially with variable drug responses. According to the present invention, certain polymorphisms in the CXCL5 gene result in increased ENA-78 production, thereby increasing neutrophil activation. Increased ENA-78 concentration can affect a patient's propensity for, and clinical course of, inflammatory diseases including pulmonary disease, lung cancer, arthritis, and other pathological states (Aras R et al., “The proinflammatory and hypercoagulable state of diabetes mellitus,” Rev Cardiovasc Med, 6:84-97 (2005); Bisset L R and Schmid-Grendelmeier P, “Chemokines and their receptors in the pathogenesis of allergic asthma: progress and perspective,” Curr Opin Pulm Med, 11:35-42 (2005); Hansson G K, “Regulation of immune mechanisms in atherosclerosis,” Ann NY Acad Sci, 947:157-65 (2001); Pettersen C A, Adler K B, “Airways inflammation and COPD: epithelialneutrophil interactions,” Chest, 121:142S-50S (2002); Robinson S C, Coussens L M, “Soluble mediators of inflammation during tumor development,” Adv Cancer Res, 93:159-87 (2005); Stockley R A, “Neutrophils and the pathogenesis of COPD,” Chest, 121:151S-5S (2002); Wislez M et al., “Upregulation of bronchioloalveolar carcinoma-derived C-X-C chemokines by tumor infiltrating inflammatory cells,” Inflamm Res, 53:4-12 (2004); Koch A E et al., “Regulation of angiogenesis by the C-X-C chemokines interleukin-8 and epithelial neutrophil activating peptide 78 in the rheumatoid joint,” Arthritis Rheum, 44:31-40 (2001); and Ross R., “Atherosclerosis is an inflammatory disease,” N Engl J Med. 340:115-26 (1999)).

The particular gene sequences of interest to the present invention comprise “mutations” or “polymorphisms” in the CXCL5 genes for the ENA-78 chemokine.

The genomes of animals and plants naturally undergo spontaneous mutation in the course of their continuing evolution (J. F. Gusella, Ann. Rev. Biochem., 55:831-854 (1986)). These mutations may be in the form of deletions, insertions, or base changes at a particular site in a nucleic acid sequence. The altered sequence and the initial sequence may co-exist in a species' population. In some instances, these changes confer neither an advantage nor a disadvantage to the species and multiple alleles of the sequence may be in stable or quasi-stable equilibrium. In some instances, however, these sequence changes will confer a survival or evolutionary advantage to the species, and accordingly, the altered allele may eventually (i.e. over evolutionary time) be incorporated into the genome of many or most members of that species.

In other instances, the altered sequence confers a disadvantage to the species, as where the mutation causes or predisposes an individual to a genetic disease. As used herein, the terms “mutation” or “polymorphism” refer to the condition in which there is a variation in the DNA sequence between some members of a species. Typically, the term “mutation” is used to denote a polymorphism that results in the gene coding for a non-functioning protein or a protein with a substantially altered or reduced function or that additionally contributes to a disease condition.

A polymorphism is thus said to be “allelic,” in that, due to the existence of the polymorphism, some members of a species carry a gene with one sequence (e.g., the original or wild-type “allele”), whereas other members may have an altered sequence (e.g., the variant or mutant “allele”). In the simplest case, only one mutated variant of the sequence may exist, and the polymorphism is said to be diallelic. The occurrence of alternative mutations can give rise to triallelic polymorphisms, etc. An allele may be referred to by the nucleotide(s) that comprise the mutation.

The terms “ENA-78” polymorphisms or “CXCL5” polymorphisms, therefore, are terms of art and refer to polymorphisms in the nucleic acid or amino acid sequence of a CXCL5 gene or gene product. For reference purposes only, GenBank Accession No. AF349466, herein incorporated by reference, is an example of a wild-type CXCL5 gene sequence. For the purposes of identifying the location of a polymorphism, the standard RefSNP cluster ID (also known as rs#) is used. Those in the art will readily recognize that nucleic acid molecules may be double-stranded molecules and that reference to a particular site on one strand refers, as well, to the corresponding site on a complementary strand. Thus, in defining a polymorphic site, reference to an adenine, a thymine (uridine), a cytosine, or a guanine at a particular site on the plus (sense) strand of a nucleic acid molecule is also intended to include the thymine (uridine), adenine, guanine, or cytosine (respectively) at the corresponding site on a minus (antisense) strand of a complementary strand of a nucleic acid molecule. Thus, reference may be made to either strand and still comprise the same polymorphic site and an oligonucleotide may be designed to hybridize to either strand. Throughout the text, in identifying a polymorphic site, reference is made to the sense strand, only for the purpose of convenience.

The preferred polymorphisms of the present invention that occur in the CXCL5 gene have been previously described (Change M S et al., “Cloning and characterization of the human neutrophil-activating peptide (ENA-78) gene,” J Biol Chem, 269:25277-82 (1994); Amoli M M et al., “Two polymorphisms in the epithelial cell-derived neutrophil-activating peptide (ENA-78) gene,” Dis Markers, 21:75-7 (2005); Zineh I et al., “Development and cross-validation of sequencing-based assays for genotyping common polymorphisms of the CXCL5 gene,” Clin Chim Acta (Epub 2006 August; 370(1-2):72).

In practicing the methods of the invention, an individual's polymorphic pattern can be established by obtaining a biological sample containing DNA from the subject and determining the sequence at predetermined polymorphic positions in the CXCL5 gene, such as those described above.

The DNA biological sample may be obtained from any nucleated cell source. Non-limiting examples of cell sources available in clinical practice include nucleated blood cells, buccal cells, cervicovaginal cells, epithelial cells from urine, fetal cells, umbilical vein cells, or any cells present in tissue obtained by biopsy or any other cell collection method. Cells may also be obtained from body fluids, including without limitation blood, saliva, sweat, urine, cerebrospinal fluid, feces, and tissue exudates at the site of infection or inflammation. DNA is extracted from the cell source or body fluid using any of the numerous methods that are standard in the art. It will be understood that the particular method used to extract DNA will depend on the nature of the source.

Determination of the sequence of the extracted DNA at polymorphic positions in the CXCL5 gene is achieved by any means known in the art, including but not limited to direct sequencing, hybridization with allele-specific oligonucleotides, allele-specific PCR, ligase-PCR, HOT cleavage, denaturing gradient gel electrophoresis (DDGE), and single-stranded conformational polymorphism (SSCP). Direct sequencing may be accomplished by any method, including without limitation chemical sequencing, using the Maxam-Gilbert method; by enzymatic sequencing, using the Sanger method; mass spectrometry sequencing; and sequencing using a chip-based technology. See, e.g., Little et al., Genet. Anal. 6:151 (1996). Preferably, DNA from a patient is first subjected to amplification by polymerase chain reaction (PCR) using specific amplification primers.

In an alternate embodiment, biopsy tissue is obtained from a patient. Antibodies that are capable of distinguishing between different polymorphic forms CXCL5 genes are then applied to samples of the tissue to determine the presence or absence of a polymorphic form specified by the antibody. The antibodies may be polyclonal or monoclonal, preferably monoclonal. Measurement of specific antibody binding to cells may be accomplished by any known method e.g. quantitative flow cytometry, or enzyme-linked or fluorescence-linked immunoassay. The presence or absence of a particular polymorphism or polymorphic pattern, and its allelic distribution (i.e., homozygosity versus heterozygosity) is determined by comparing the values obtained from a patient with norms established from populations of patients having known polymorphic patterns.

In an alternate embodiment, RNA is isolated from biopsy tissue using standard methods well known to those of ordinary skill in the art such as guanidium thiocyanate-phenol-chloroform extraction (Chomocyznski et al., 1987, Anal. Biochem., 162:156.) The isolated RNA is then subjected to coupled reverse transcription and amplification by polymerase chain reaction (RT-PCR), using specific oligonucleotide primers. Conditions for primer annealing are chosen to ensure specific reverse transcription and amplification; thus, the appearance of an amplification product is diagnostic of the presence of particular alleles. In another embodiment, RNA is reverse-transcribed and amplified, after which the amplified sequences are identified by, e.g., direct sequencing.

In practicing the present invention, the distribution of polymorphic patterns in a large number of individuals exhibiting particular markers of immunological status is determined by any of the methods described above, and compared with the distribution of polymorphic patterns in patients that have been matched for age, ethnic origin, and/or any other statistically or medically relevant parameters, who exhibit quantitatively or qualitatively different status markers. Correlations are achieved using any statistical method known in the art, including but not limited to nominal logistic regression or standard least squares regression analysis. In this manner, it is possible to establish statistically significant correlations between particular polymorphic patterns and particular cardiovascular statuses. It is further possible to establish statistically significant correlations between particular polymorphic patterns and changes in immunological status such as, would result, e.g., from particular treatment regimens. In this manner, it is possible to correlate polymorphic patterns with responsiveness to particular treatments.

Polymorphic positions in the CXCL5 gene encoding ENA-78, which are encompassed by the invention, are identified by determining the DNA sequence of all or part of the CXCL5 genes in a multiplicity of individuals in a population. DNA sequence determination may be achieved using any conventional method, including, e.g., chemical or enzymatic sequencing.

Contemplated single nucleotide polymorphisms (SNPs) include any validated SNPs found in SNP databases such as, but not limited to, NCBI dbSNP, SeattleSNPs, and PharmGKB. Examples of contemplated SNPS include, but are not limited to, the following NCBI SNP ID Nos.: rs425535; rs352046; rs3775488; rs352047; rs2437285; rs16850352; rs352045; rs12512838; rs454618; rs16850345; rs17813879; rs16850354; rs12505025; rs16850337; rs11551733; rs3211021; rs2437283; rs34057204; rs3211020; rs34648742; rs7693610; rs2437284; rs3864158; rs35273633; rs35811098; rs4379035; rs34160952; rs34445376; rs34721804; rs35883103; rs34249049; rs34386106; rs1540413; and rs2458099, all of which are herein incorporated by reference (Table 1). CXCL5 single nucleotide polymorphisms available in Applied Biosystems data source, including the following: rs13143127; rs2437282; rs2564594; rs416046; rs7655764; rs16850358; rs2457996; rs352009; rs420389; rs7697034; rs16850359; rs2458093; rs352010; rs426901; rs16850360; rs2458094; rs3756075; rs183028; rs2458096; rs377579; rs450373; rs187080; rs2458098; rs3910390; rs551545; rs2435197; rs2472649; rs409336; and rs6818834, are also contemplated herein and incorporated by reference (see attached Appendix A with details as provided on the NCBI electronic database).

TABLE 1
Single Nucleotide Polymorphisms of CXCL5 gene

| | | | | | | | | | Allele | |
|---|---|---|---|---|---|---|---|---|---|---|
| Genomic Data | | | | | | | | | Frequencies | |
| | | | | | AA | | | Allele | | Total |
| SNP ID | Valid | Chr 4 pos | Sequence | Recs | Chg | Type | Recs | freq | Pop | sample |
| rs425535 | C, F, O | 75082861(−) | 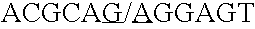 | 1 | Q/Q | syn ese | 13 | 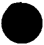 | MN NA EU EA WA | 1182 |
| rs3775488 | C, F, A | 75081716(+) | 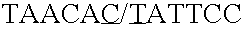 | 1 | — | utr | 6 | 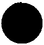 | EA NA | 394 |
| rs352047 | F, A | 75080954(−) | 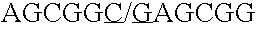 | 1 | — | utr ese | 1 | 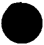 | MN | 184 |
| rs2437285 | C, F, O, A | 75079102(−) | 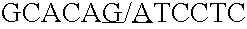 | 1 | — | loc | 3 | 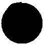 | NA | 142 |
| rs16850352 | F | 75083938(+) | 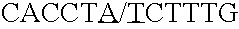 | 1 | — | loc | 7 | 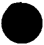 | NA EU EA WA | 560 |
| rs352045 | F | 75083551(−) | 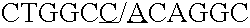 | 1 | — | loc | 4 | 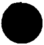 | EU EA WA | 406 |
| rs12512838 | A | 75078267(−) | 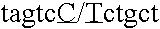 | 1 | — | loc | 0 | — | — | — |
| rs454618 | F | 75079973(−) | 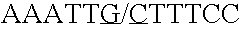 | 1 | — | loc | 6 | 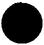 | EA NA EU WA | 444 |
| rs16850345 | F | 75079788(+) | 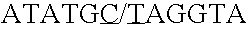 | 1 | — | loc | 7 | 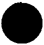 | NA EU EA WA | 562 |
| rs17813879 | F | 75079004(+) | 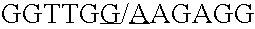 | 1 | — | loc | 3 | 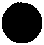 | NA | 142 |
| rs16850354 | F | 75084824(+) | 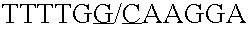 | 1 | — | loc | 7 | 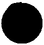 | NA EU EA WA | 562 |
| rs12505025 | A | 75078341(+) | 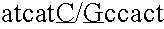 | 1 | — | loc | 0 | — | — | — |
| rs16850337 | F | 75078731(+) | 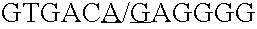 | 1 | — | loc | 3 | 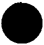 | NA | 142 |
| rs352046 | A | 75083414(−) | 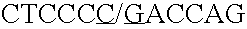 | 1 | — | loc tfbs | 0 | — | — | — |
| rs11551733 | — | 75083130(−) | 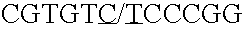 | 1 | V/V | syn | 0 | — | — | — |
| rs3211021 | — | 75080536(−) | 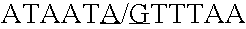 | 1 | — | utr | 0 | — | — | — |
| rs2437283 | — | 75081849(−) | 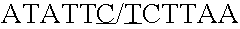 | 1 | — | utr | 0 | — | — | — |
| rs34057204 | — | 75081658(+) | 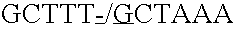 | 1 | — | utr | 0 | — | — | — |
| rs3211020 | — | 75080546(−) | 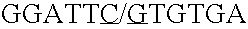 | 1 | — | utr ese | 0 | — | — | — |
| rs34648742 | — | 75082190(+) | 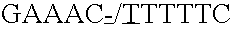 | 1 | — | utr | 0 | — | — | — |
| rs7693610 | — | 75083219(+) | 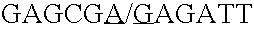 | 1 | — | utr ese | 0 | — | — | — |
| rs2437284 | — | 75081670(−) | 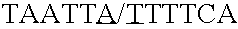 | 1 | — | utr | 0 | — | — | — |
| rs3864158 | — | 75082719(+) | 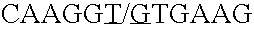 | 1 | — | int | 4 | 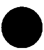 | EU EA WA | 420 |
| rs35273633 | — | 75078603(+) | 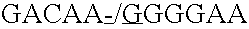 | 1 | — | loc | 0 | — | — | — |
| rs35811098 | — | 75078405(+) | 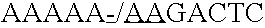 | 1 | — | loc | 0 | — | — | — |
| rs4379035 | — | 75083776(+) | 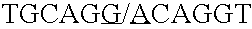 | 1 | — | loc | 4 | 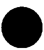 | EU EA WA | 416 |
| rs34160952 | — | 75079162(+) | 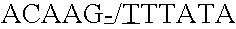 | 1 | — | loc | 0 | — | — | — |
| rs34445376 | — | 75078385(+) | 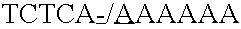 | 1 | — | loc | 0 | — | — | — |
| rs34721804 | — | 75078396(+) | 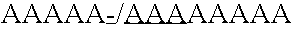 | 1 | — | loc | 0 | — | — | — |
| rs35883103 | — | 75079662(+) | 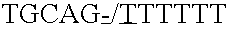 | 1 | — | loc | 0 | — | — | — |
| rs34249049 | — | 75079458(+) | 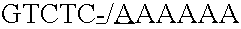 | 1 | — | loc | 0 | — | — | — |
| rs34386106 | — | 75079457(+) | 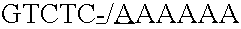 | 1 | — | loc | 0 | — | — | — |
| rs1540413 | — | 75083994(−) | 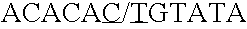 | 1 | — | loc | 4 | 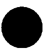 | EU EA WA | 418 |
| rs2458099 | — | 75079240(+) | 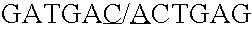 | 1 | — | loc | 4 | 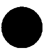 | EU EA WA | 420 |
| rs497427 | — | 75079702(+) | 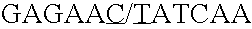 | 1 | — | loc | 4 | 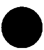 | EU EA WA | 416 |

Preferably, the present invention is directed to single nucleotide polymorphisms in the CXCL5 gene that affect the regulation of ENA-78 protein levels in the body, Specifically, two single nucleotide polymorphism (SNPs), known as the promoter −156G/C (rs352046) and exonic 398G/A (rs425535), which have been shown to occur with relatively high allele frequencies in largely European and United States populations, are used herein.

The present invention also provides isolated nucleic acids comprising the polymorphic positions described above for the human CXCL5 gene; vectors comprising the nucleic acids; and transformed host cells comprising the vectors. The invention also provides probes which are useful for detecting these polymorphisms.

In practicing the present invention, many conventional techniques in molecular biology, microbiology, and recombinant DNA, are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Ausubel et al., Current Protocols in Molecular Biology, 1997, (John Wiley and Sons); and Methods in Enzymology Vol. 154 and Vol. 155 (Wu and Grossman, and Wu, eds., respectively).

Insertion of nucleic acids (typically DNAs) comprising the sequences of the present invention into a vector is easily accomplished when the termini of both the DNAs and the vector comprise compatible restriction sites. If this cannot be done, it may be necessary to modify the termini of the DNAs and/or vector by digesting back single-stranded DNA overhangs generated by restriction endonuclease cleavage to produce blunt ends, or to achieve the same result by filling in the single-stranded termini with an appropriate DNA polymerase.

Alternatively, any site desired may be produced, e.g., by ligating nucleotide sequences (linkers) onto the termini. Such linkers may comprise specific oligonucleotide sequences that define desired restriction sites. Restriction sites can also be generated by the use of the polymerase chain reaction (PCR). See, e.g., Saiki et al., 1988, Science 239:48. The cleaved vector and the DNA fragments may also be modified if required by homopolymeric tailing.

The nucleic acids may be isolated directly from cells or may be chemically synthesized using known methods. Alternatively, the polymerase chain reaction (PCR) method can be used to produce the nucleic acids of the invention, using either chemically synthesized strands or genomic material as templates. Primers used for PCR can be synthesized using the sequence information provided herein and can further be designed to introduce appropriate new restriction sites, if desirable, to facilitate incorporation into a given vector for recombinant expression.

The nucleic acids of the present invention may be flanked by native CXCL5 gene sequences, or may be associated with heterologous sequences, including promoters, enhancers, response elements, signal sequences, polyadenylation sequences, introns, 5′- and 3′-noncoding regions, and the like. The nucleic acids may also be modified by many means known in the art. Non-limiting examples of such modifications include methylation, “caps”, substitution of one or more of the naturally occurring nucleotides with an analog, internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.).

Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. PNAs are also included. The nucleic acid may be derivatized by formation of a methyl or ethyl phosphotriester or an alkyl phosphoramidate linkage. Furthermore, the nucleic acid sequences of the present invention may also be modified with a label capable of providing a detectable signal, either directly or indirectly. Exemplary labels include radioisotopes, fluorescent molecules, biotin, and the like.

The invention also provides nucleic acid vectors comprising the disclosed CXCL5 gene sequences or derivatives or fragments thereof. A large number of vectors, including plasmid and fungal vectors, have been described for replication and/or expression in a variety of eukaryotic and prokaryotic hosts, and may be used for gene therapy as well as for simple cloning or protein expression. Non-limiting examples of suitable vectors include without limitation pUC plasmids, pET plasmids (Novagen, Inc., Madison, Wis.), or pRSET or pREP (Invitrogen, San Diego, Calif.), and many appropriate host cells, using methods disclosed or cited herein or otherwise known to those skilled in the relevant art. The particular choice of vector/host is not critical to the practice of the invention.

Suitable host cells may be transformed/transfected/infected as appropriate by any suitable method including electroporation, CaCl₂ mediated DNA uptake, fungal or viral infection, microinjection, microprojectile, or other established methods. Appropriate host cells included bacteria, archebacteria, fungi, especially yeast, and plant and animal cells, especially mammalian cells. A large number of transcription initiation and termination regulatory regions have been isolated and shown to be effective in the transcription and translation of heterologous proteins in the various hosts. Examples of these regions, methods of isolation, manner of manipulation, etc. are known in the art.

Nucleic acids encoding CXCL5-derived gene sequences may be introduced into cells by recombination events. For example, such a sequence can be introduced into a cell and thereby effect homologous recombination at the site of an endogenous gene or a sequence with substantial identity to the gene. Other recombination-based methods such as nonhomologous recombinations or deletion of endogenous genes by homologous recombination may also be used.

The nucleic acids of the present invention find use as probes for the detection of genetic polymorphisms and as templates for the recombinant production of normal or variant ENA-78.

Probes in accordance with the present invention comprise without limitation isolated nucleic acids of about 10-100 bp, preferably 15-75 bp and most preferably 17-25 bp in length, which hybridize at high stringency to CXCL5 gene-derived polymorphic sequences disclosed herein or to a sequence immediately adjacent to a polymorphic position. Furthermore, in some embodiments a full-length gene sequence may be used as a probe. In one series of embodiments, the probes span the polymorphic positions in the CXCL5 genes disclosed above. In another series of embodiments, the probes correspond to sequences immediately adjacent to the polymorphic positions.

The present invention provides materials and methods for detecting the existence of genetic polymoprhisms within patient genes encoding ENA-78, which can be used to assess patient immunological status. In accordance with the invention, the polymorphic pattern of CXCL5 sequences in a patient can predict the responsiveness of the patient to particular therapeutic interventions and serve as an indicator of predisposition to various forms of inflammatory disease.

The present invention provides diagnostic methods for assessing immunological status in a patient. Immunological status as used herein refers to the physiological status of an individual's immune system as reflected in one or more markers or indicators. Status markers include, without limitation, clinical or physiological measurements such as, e.g., white blood cell (i.e., leukocyte) counts; granulocyte counts, lymphocyte counts, monocyte/macrophage counts, ENA-78 protein concentrations, ENA-78 mRNA or cDNA concentrations, CXCL5 genotypes, carotid intima media thickness, blood pressure, lipoprotein levels, and C-reactive protein levels. Status markers according to the invention include diagnoses of one or more inflammatory diseases. It will be understood that a diagnosis of an inflammatory disease made by a medical practitioner encompasses clinical measurements and medical judgment. Status markers according to the invention are assessed using conventional methods well known in the art. Also included in the evaluation of immunological status are quantitative or qualitative changes in status markers with time, such as would be used, e.g., in the determination of a patient's response to a particular therapeutic regimen.

The polymorphisms of the present invention are used in the diagnosis/prognosis of disease associated with dysregulation of CXCL5 or ENA-78 or, alternatively, they are used to predict a patient's responsiveness to therapeutic interventions in the treatment of diseases associated with dysregulation of CXCL5 or ENA-78. Diseases associated with dysregulation of CXCL5 or ENA-78 include, but are not limited to, cancer (angiogenic tumors), asthma, bowel disease, arthritis, and those diseases listed in Table 2.

TABLE 2
Diseases associated with dysregulation of CXCL5 or ENA-78

| | Disease | Characteristic | Journal | Author | Title |
|---|---|---|---|---|---|
| Cancer | Gastric Cancer | protein upregulated | J Cancer Res Clin Oncol. 2007 May 4 | Park | CXCL5 overexpression is |
| | | | | | associated with late stage gastric |
| | | | | | cancer. |
| | AML | expression upregulated in subset | Haematologica. 2007 Mar; 92(3): 332-41 | Bruserod | Subclassification of patients with |
| | | | | | acute myelogenous leukemia based |
| | | | | | on chemokine responsiveness and |
| | | | | | constitutive chemokine release by |
| | | | | | their leukemic cells. |
| | Non-small Cell Lung Cancer | Protein upregulated in | J Clin Invest. 1998 Aug 1; 102(3): 465-72 | Arenberg | Epithelial-neutrophil activating |
| | | human: mouse model ENA | | | peptide (ENA-78) is an important |
| | | associated with growing | | | angiogenic factor in non-small cell |
| | | tumor | | | lung cancer |
| | Malignant Nasopharyngeal epithelium | expression downregulated | Life Sci. 2000 Jul | Fung | Differential gene expression in |
| | | | 14; 67(8): 923-36 | | nasopharyngeal carcinoma cells |
| Arthritic | Arthritis | | J Immunol. 1999 Jun | Halloran | The role of an epithelial neutrophil- |
| | | | 15; 162(12): 7492-500 | | activating peptide-78-like protein in |
| | | | | | rat adjuvant-induced arthritis. |
| | R. Arthritis | protein upregulated in | Rheumatol Int. 2005 Dec; 26(2): 162-7 | Erdem | Different ELR (+) angiogenic CXC |
| | | synovial fluid | | | chemokine profiles in synovial fluid |
| | | | | | of patients with Behcet's disease, |
| | | | | | familial Mediterranean fever, |
| | | | | | rheumatoid arthritis, and |
| | | | | | osteoarthritis. |
| | | | J Clin Invest. 1994 Sep; 94(3): 1012-8. | Koch | Epithelial neutrophil activating |
| | | | | | peptide-78: a novel chemotactic |
| | | | | | cytokine for neutrophils in arthritis |
| | | | Springer Semin | Szekanecz | Chemokines in rheumatoid arthritis |
| | | | Immunopathol. 1998; 20(1-2): | | |
| | | | 115-32. | | |
| | | Protein upregulated and | J Immunol. 2000 Sep 1; 165(5): 2755-63 | Woods | Reduction of inflammatory cytokines |
| | | down regulated by IL-13 | | | and prostaglandin E2 by IL-13 gene |
| | | | | | therapy in rheumatoid arthritis |
| | | | | | synovium |
| | | protein upregulated in | Arthritis Rheum. 2001 Jan; 44(1): 31-40 | Koch | Regulation of angiogenesis by the |
| | | synovial fluid | | | C-X-C chemokines interleukin-8 and |
| | | | | | epithelial neutrophil activating |
| | | | | | peptide 78 in the rheumatoid joint. |
| Lungs | Asthma | expression upregulated | Thorax. 2007 Mar 21 | Qiu | Bronchial mucosal inflammation |
| | | | | | and up-regulation of CXC |
| | | | | | chemoattractants and receptors in |
| | | | | | severe exacerbations of asthma. |
| | Cystic Fibrosis | expression upregualted | Am J Respir Cell Mol Biol. | Bozic | Receptor binding specificity and |
| | | | 1996 Mar; 14(3): 302-8 | | pulmonary gene expression of the |
| | | | | | neutrophil-activating peptide ENA- |
| | | | | | 78 |
| | Idiopathic pulmonary fibrosis | elevated protein: angiogenic properties | Am J Respir Crit Care Med. | Keane | ENA-78 is an important |
| | | | 2001 Dec 15; 164(12): 2239-42 | | angiogenic factor in idiopathic |
| | | | | | pulmonary fibrosis. |
| | Emphysema | No association (just IL-8) | Thorax. 2002 | Tanino | Increased levels of interleukin-8 in |
| | | | May; 57(5): 405-11 | | BAL fluid from smokers |
| | | | | | susceptible to pulmonary |
| | | | | | emphysema. |
| | Respiratory Distress | | Chest. 1994 Mar; 105(3 | Donnelly | Chemotactic cytokines in the |
| | | | Suppl): 98S-99S | | established adult respiratory |
| | | | | | distress syndrome and at-risk |
| | | | | | patients. |
| | Acute Respiratory Distress Syndrome | elevated in bronchoalveolar | Am J Respir Crit Care Med. | Goodman | Inflammatory cytokines in patients |
| | | lavage-associated with high | 1996 Sep; 154(3 Pt 1): 602-11 | | with persistence of the acute |
| | | PMNs | | | respiratory distress syndrome. |
| | ARDS: Sepsis | downregulated CXCR2 (ENA receptor) | J Immunol. 1999 Feb | Cummings | Expression and function of the |
| | | | 15; 162(4): 2341-6 | | chemokine receptors CXCR1 and |
| | | | | | CXCR2 in sepsis.Cummings CJ, |
| | | | | | Martin TR, Frevert CW, Quan JM, |
| | | | | | Wong VA, Mongovin SM |
| Liver | ischemia/reperfusion-induced lung injury | rat: protein upregulated | J Clin Invest. 1995 | Colletti | Chemokine expression during |
| | | | Jan; 95(1): 134-41 | | hepatic ischemia/reperfusion- |
| | | | | | induced lung injury in the rat. The |
| | | | | | role of epithelial neutrophil |
| | | | | | activating protein. |
| | hepatic ischemia/reperfusion | rat: protein upregulated | Hepatology. 1996 | Colletti | The role of cytokine networks in |
| | | | Mar; 23(3): 506-14 | | the local liver injury following |
| | | | | | hepatic ischemia/reperfusion in |
| | | | | | the rat. |
| | | rat: protein upregulated | Shock. 1996 May; 5(5): 371-7 | Colletti | Post-ischemic shunt following |
| | | | | | hepatic ischemia/reperfusion does |
| | | | | | not affect tissue chemokine levels |
| | | | | | of tissue injury |
| | | | Hepatology. 2000 | Colletti | The ratio of ELR+ to ELR− CXC |
| | | | Feb; 31(2): 435-45 | | chemokines affects the lung and |
| | | | | | liver injury following hepatic |
| | | | | | ischemia/reperfusion in the rat |
| | | | Shock. 2001 Oct; 16(4): 312-9. | Colletti | Lung and liver injury following |
| | | | Links | | hepatic ischemia/reperfusion in |
| | | | | | the rat is increased by exogenous |
| | | | | | lipopolysaccharide which also |
| | | | | | increases hepatic TNF production |
| | | | | | in vivo and in vitro. |
| | Hepatectomy: inflammation following | rat: protein upregulated | Shock. 1996 Dec; 6(6): 397-402 | Colletti | Hepatic inflammation following |
| | | | | | 70% hepatectomy may be related |
| | | | | | to up-regulation of epithelial |
| | | | | | neutrophil activating protein-78. |
| | | commentary on Shock. 1996 | Shock. 1996 Dec; 6(6): 403-4 | Jaeschke | Chemokines, neutrophils, and |
| | | Dec; 6(6): 397-402 rat: associated with | | | inflammatory liver injury |
| | | hepatocyte proliferation | Shock. 1998 Oct; 10(4): 248-57 | Colletti | Proliferative effects of CXC |
| | | | | | chemokines in rat hepatocytes in |
| | | | | | vitro and in vivo |
| | Acetaminophen induced hepatotoxicity | rat: ENA decreased histological and | FASEB J. 1999 | Hogaboam | Novel CXCR2-dependent liver |
| | | markers of hepatic injury | Sep; 13(12): 1565-74 | | regenerative qualities of ELR- |
| | | | | | containing CXC chemokines |
| Renal | Renal allograft rejection | expression upregulated | Transplantation. 1995 Jan | Schmouder | Epithelial-derived neutrophil- |
| | | | 15; 59(1): 118-24 | | activating factor-78 production in |
| | | | | | human renal tubule epithelial cells |
| | | | | | and in renal allograft rejection. |
| | Adrenocortical carcinoma | 1 patient overexpressed | J Clin Endocrinol Metab. | Schteingart | Overexpression of CXC |
| | | protein; mouse with ENA | 2001 Aug; 86(8): 3968-74 | | chemokines by an adrenocortical |
| | | antisera had marked | | | carcinoma: a novel clinical |
| | | decreased tumor growth | | | syndrome. |
| Bowels | Pancreatitis | expression upregulated | Gastroenterology. 2000 | Saurer | Differential expression of |
| | | | Feb; 118(2): 356-67 | | chemokines in normal pancreas |
| | | | | | and in chronic pancreatitis |
| | | elevated protein | Br J Surg. 2002 | Shokuhi | Levels of the chemokines growth- |
| | | | May; 89(5): 566-72 | | related oncogene alpha and |
| | | | | | epithelial neutrophil-activating |
| | | | | | protein 78 are raised in patients |
| | | | | | with severe acute pancreatitis. |
| | Ulcerative Colitis | expression upregulated | Am J Physiol. 1997 | Keates | Enterocytes are the primary |
| | | | Jul; 273(1 Pt 1): G75-82 | | source of the chemokine ENA-78 |
| | | | | | in normal colon and ulcerative |
| | | | | | colitis. |
| | IBD | expression and protein | Gastroenterology. 1997 | Z'Graggen | The C-X-C chemokine ENA-78 is |
| | | upregulated | Sep; 113(3): 808-16 | | preferentially expressed in |
| | | | | | intestinal epithelium in |
| | | | | | inflammatory bowel disease. |
| Infection | Microsporidian infection | expression upregulated | Infect Immun. 2007 | Fischer | Induction of host chemotactic |
| | | | Apr; 75(4): 1619-25 | | response by Encephalitozoon |
| | | | | | spp. |
| | Urosepsis | slight elevation in urine | J Infect Dis. 2000 | Olszyna | Chemotactic activity of CXC |
| | | | Dec; 182(6): 1731-7. Epub | | chemokines interleukin-8, growth- |
| | | | 2000 Oct 23 | | related oncogene-alpha, and |
| | | | | | epithelial cell-derived neutrophil- |
| | | | | | activating protein-78 in urine of |
| | | | | | patients with urosepsis. |
| | H Pylori Gastritis | H Pylori LPS stimulates ENA release | Infect Immun. 1998 | Bliss | Helicobacter pylori |
| | | | Nov; 66(11): 5357-63 | | lipopolysaccharide binds to CD14 |
| | | | | | and stimulates release of |
| | | | | | interleukin-8, epithelial neutrophil- |
| | | | | | activating peptide 78, and |
| | | | | | monocyte chemotactic protein 1 |
| | | | | | by human monocytes. |
| | | expression upregulated in patients | Infect Immun. 2001 | Rieder | Comparison of CXC chemokines |
| | | | Jan; 69(1): 81-8 | | ENA-78 and interleukin-8 |
| | | | | | expression in Helicobacter pylori- |
| | | | | | associated gastritis |
| | | | J Clin Pathol. 2001 | Shimoyama | Influence of smoking and alcohol |
| | | | Apr; 54(4): 332-4 | | on gastric chemokine mRNA |
| | | | | | expression in patients with |
| | | | | | Helicobacter pylori infection. |
| CVD | Congestive Heart Failure | protein upregulated | Cardiovasc Res. 2000 Jan | Damas | CXC-chemokines, a new group of |
| | | | 14; 45(2): 428-36 | | cytokines in congestive heart |
| | | | | | failure-possible role of platelets |
| | | | | | and monocytes |
| | Hyperhomocysteinemic | elevated protein; reduced with folic acid | Arterioscler Thromb Vasc | Holven | Folic acid treatment reduces |
| | | | Biol. 2002 Apr 1; 22(4): 699-703 | | chemokine release from |
| | | | | | peripheral blood mononuclear |
| | | | | | cells in hyperhomocysteinemic |
| | | | | | subjects. |
| | Chronic Prostate Conditions | protein upregulated | Urology. 2000 Dec | Hochreiter | Evaluation of the cytokines |
| | | | 20; 56(6): 1025-9 | | interleukin 8 and epithelial |
| | | | | | neutrophil activating peptide 78 as |
| | | | | | indicators of inflammation in |
| | | | | | prostatic secretions |
| | Major trauma: burns and tissue injury | protein upregulated | Eur J Surg. 1996 | Schinkel | Kinetics of circulating adhesion |
| | | | Oct; 162(10): 763-8 | | molecules and chemokines after |
| | | | | | mechanical trauma and burns. |
| | Hyper-IgE syndrome | decreased expression | Clin Immunol. 2001 | Chehimi | Cytokine and chemokine |
| | | | Jul; 100(1): 49-56 | | dysregulation in hyper-IgE |
| | | | | | syndrome. |
| | Premature neonates | elevated protein compared to adults and full term | Pediatr Res. 2002 | Sullivan (UF) | Circulating concentrations of |
| | | | May; 51(5): 653-7 | | chemokines in cord blood, |
| | | | | | neonates, and adults |

In one embodiment, therapeutic treatments are aimed at the elimination or amelioration of symptoms and events associated with inflammatory disease. Such treatments include without limitation one or more of alteration in diet, lifestyle, and exercise regimen; invasive and noninvasive surgical techniques such as atherectomy, angioplasty with or without stent placement, and coronary bypass surgery; and pharmaceutical interventions, such as administration of ACE inhibitors, angiotensin II receptor antagonists, diuretics, alpha-adrenoreceptor antagonists, cardiac glycosides, phosphodiesterase inhibitors, beta1-adrenoreceptor antagonists, beta-2 adrenoreceptor agonists, leukotriene receptor antagonists, calcium channel blockers, HMG-CoA reductase inhibitors, bile acid sequestrants, fibric acid derivatives, thiazolidinediones, peroxisome proliferator-activated receptor agonists and antagonists, biguanides, imidazoline receptor blockers, endothelin receptor blockers, CETP inhibitors, non-steroidal anti-inflammatory agents (e.g., aspirin, COX-2 inhibitors, ibuprofen and the like), immunologics, and organic nitrites. Interventions with pharmaceutical agents not yet known whose activity correlates with particular polymorphic patterns associated with cardiovascular disease are also encompassed.

A non-limiting list of inflammatory diseases that are directly caused by inflammatory cytokines and can be diagnosed/prognosed in accordance with the present invention include, but are not limited to: arthritis where inflammatory cytokines destroy lead to lesion in the synovial membrane and destruction of joint cartilage and bone; kidney failure where inflammatory cytokines restrict circulation and damage nephrons; lupus wherein inflammatory cytokines induce an autoimmune attack; asthma where inflammatory cytokines close the airway; psoriasis where inflammatory cytokines induce dermatitis; pancreatitis where inflammatory cytokines induce pancreatic cell injury; allergy where inflammatory cytokines induce autoimmune reactions; fibrosis where inflammatory cytokines attack traumatized tissue; surgical complications where inflammatory cytokines prevent healing; anemia where inflammatory cytokines attack erythropoietin production; and fibromyalgia where inflammatory cytokines are elevated in fibromyalgia patients.

Other diseases associated with chronic inflammation include cancer, which is typified by chronic inflammation; heart attack where chronic inflammation contributes to coronary atherosclerosis; Alzheimer's disease where chronic inflammation destroys brain cells; congestive heart failure where chronic inflammation compromising heart muscle pumping ability; stroke where chronic inflammation promotes thrombo-embolic events; and aortic valve stenosis where chronic inflammation damages heart valves. Arteriosclerosis, osteoporosis, Parkinson's disease, infection, inflammatory bowel disease including Crohn's disease and ulcerative colitis as well as multiple sclerosis (a typical autoimmune inflammatory-related disease), lupus, diabetes, chronic obstructive pulmonary disease, psoriasis, and scleroderma are also related to inflammation. Some diseases in advanced stages can be life threatening.

The present invention provides kits for the determination of the sequence at polymorphic positions within the CXCL5 genes in a patient. The kits comprise a means for determining the sequence at one or more polymorphic positions, and may optionally include data for analysis of polymorphic patterns. The means for sequence determination may comprise suitable nucleic acid-based reagents. Preferably, the kits also comprise suitable buffers, control reagents where appropriate, and directions for determining the sequence at a polymorphic position. The kits may also comprise data for correlation of particular polymorphic patterns with desirable treatment regimens or other indicators.

The invention provides nucleic acid-based methods for detecting polymorphic patterns in a biological sample. The sequence at particular polymorphic positions in the CXCL5 genes encoding ENA-78 is determined using any suitable means known in the art, including without limitation hybridization with polymorphism-specific probes and direct sequencing.

The present invention also provides kits suitable for nucleic acid-based diagnostic applications. In one embodiment, diagnostic kits include the following components: (i) probe DNA: the probe DNA may be pre-labelled; alternatively, the probe DNA may be unlabelled and the ingredients for labelling may be included in the kit in separate containers; and (ii) hybridization reagents: the kit may also contain other suitably packaged reagents and materials needed for the particular hybridization protocol, including solid-phase matrices, if applicable, and standards.

In another embodiment, diagnostic kits include: (i) sequence determination primers: sequencing primers may be pre-labelled or may contain an affinity purification or attachment moiety; and (ii) sequence determination reagents: the kit may also contain other suitably packaged reagents and materials needed for the particular sequencing protocol. In one preferred embodiment, the kit comprises a panel of sequencing primers, whose sequences correspond to sequences adjacent to the SNPs described above, as well as a means for detecting the presence of each polymorphic sequence.

The kits referred to above may include instructions for conducting the test. Furthermore, in preferred embodiments, the diagnostic kits are adaptable to high-throughput and/or automated operation.

The present invention also provides methods for the treatment and/or prevention of inflammatory disease upon detection of CXCL5 gene polymorphisms, as described herein.

In one embodiment, compounds are provided in an amount to inhibit ENA-78 protein production. In one embodiment, the compound is preferably in an amount to inhibit at least 25% production of ENA-78: More preferably at least 40% production of ENA-78 is inhibited and most preferably 50% or more is inhibited.

In another embodiment, ENA-78 concentrations are modified by administering to the patient an effective amount of a non-polymorphic CXCL5 gene. Preferably, the non-polymorphic CXCL5 gene is contained in an appropriate expression vehicle. Such expression vehicles include, but are not limited to, plasmids, eukaryotic vectors, prokaryotic vectors (such as, for example, bacterial vectors), and viral vectors. In one embodiment, the vector is a viral vector. Viral vectors which may be employed include RNA virus vectors (such as retroviral vectors, including lentiviral vectors) and DNA virus vectors (such as adenoviral vectors, adeno-associated virus vectors, Herpes Virus vectors, and vaccinia virus vectors). When a DNA virus vector is employed in constructing the vector, the CXCL5 gene is in the form of DNA. When an RNA virus vector is employed in constructing the vector, the CXCL5 gene is in the form of RNA. Preferable viral vectors include adenoviral vectors (preferably lacking all viral genes, i.e. high capacity or gutless), lentiviral vectors (e.g. HIV, BIV-based), and adeno-associated virus (AAV) vectors.

In one embodiment, the viral vector including the non-polymorphic CXCL5 gene is an adenoviral vector.

The adenoviral vector which is employed may, in one embodiment, be an adenoviral vector which includes essentially the complete adenoviral genome (Shenk et al., Curr. Top. Microbiol. Immunol., 111(3): 1-39 (1984). Alternatively, the adenoviral vector may be a modified adenoviral vector in which at least a portion of the adenoviral genome has been deleted.

In a preferred embodiment, the adenoviral vector comprises an adenoviral 5′ ITR; an adenoviral 3′ ITR; an adenoviral encapsidation signal; a non-polymorphic CXCL5 DNA sequence, and a promoter controlling the non-polymorphic CXCL5 gene expression. The vector is free of at least the majority of adenoviral E1 and E3 DNA sequences, but is not free of all of the E2 and E4 DNA sequences, and DNA sequences encoding adenoviral proteins promoted by the adenoviral major late promoter. In one embodiment, the vector also is free of at least a portion of at least one DNA sequence selected from the group consisting of the E2 and E4 DNA sequences.

In another embodiment, the vector is free of at least the majority of the adenoviral E1 and E3 DNA sequences, and is free of a portion of the other of the E2 and E4 DNA sequences.

In still another embodiment, the gene in the E2a region that encodes the 72 kilodalton binding protein is mutated to produce a temperature sensitive protein that is active at 32° C., the temperature at which the viral particles are produced. This temperature sensitive mutant is described in Ensinger, et al., J. Virology, 10:328-339 (1972), Van der Vliet et al., J. Virology, 15:348-354 (1975), and Friefeld, et al., Virology, 124:380-389 (1983).

Such a vector, in a preferred embodiment, is constructed first by constructing, according to standard techniques, a shuttle plasmid which contains, beginning at the 5′ end, the “critical left end elements,” which include an adenoviral 5′ ITR, an adenoviral encapsidation signal, and an E1a enhancer sequence; a promoter (which may be an adenoviral promoter or a foreign promoter); a multiple cloning site (which may be as herein described); a poly A signal; and a DNA segment which corresponds to a segment of the adenoviral genome. The promoter may, in one embodiment, be a regulatable promoter, such as, for example, a glucocorticoid-responsive promoter or an estrogen-responsive promoter, or the promoter may be a tissue-specific promoter.

The vector also may, in another embodiment, contain genomic elements which may increase and/or maintain expression of the non-polymorphic CXCL5 DNA sequence. Such genomic elements include, but are not limited to, introns, exons, polyadenylation sequences, and 5′ and 3′ untranslated regions. Such genomic elements, and representative examples thereof, also are described in U.S. Pat. No. 5,935,935, issued Aug. 10, 1999. The vector also may contain a tripartite leader sequence. The DNA segment which corresponds to a segment of the adenoviral genome serves as a substrate for homologous recombination with an adenovirus. The plasmid may also include a selectable marker and an origin of replication. The origin of replication may be a bacterial origin of replication. Representative examples of such shuttle plasmids include pAvS6, which is described in published PCT Application Nos. WO 94/23582, published Oct. 27, 1994, and WO 95/09654, published Apr. 13, 1995, and in U.S. Pat. No. 5,543,328, issued Aug. 6, 1996. The non-polymorphic CXCL5 gene then may be inserted into the multiple cloning site of the shuttle plasmid to produce a plasmid vector.

This construct is then used to produce an adenoviral vector. Homologous recombination may be effected through co-transfection of the plasmid vector and the adenovirus into a helper cell line, such as 293 cells, by CaPO₄ precipitation. Upon such homologous recombination, a recombinant adenoviral vector is formed that includes DNA sequences derived from the shuttle plasmid between the 5′ITR and the homologous recombination fragment, and the DNA derived from the adenovirus between the homologous recombination fragment and the 3′ ITR.

The adenoviral vector of the present invention may be administered to a patient in vivo in an amount effective to promote decreased ENA-78 production in the patient. In another alternative, the retroviral vectors hereinabove described, or a polynucleotide encoding for normal ENA-78 production, or an analogue, fragment, or derivative thereof, may be encapsulated within liposomes. The liposomes, which encapsulate the retroviral vectors or a polynucleotide encoding for normal ENA-78 production, or analogue, fragment, or derivative thereof, may be administered to a host in conjunction with a pharmaceutical carrier.

In an alternative embodiment, retroviral producer cells, such as those derived from the packaging cell lines that include a non-polymorphic CXCL5 gene, may be administered to an animal. Such producer cells may, in one embodiment, be administered systemically (e.g., intravenously or intraarterially). The producer cell line then produces retroviral vectors including a polynucleotide comprising the non-polymorphic CXCL5 gene.

Another embodiment has the expression of ENA-78 controlled by an inducible promoter. The use of an inducible gene expression system would allow the precise regulation of ENA-78 in a reversible manner. Several inducible systems are currently available. One example of a controlled promoter system in the Tet-On™ and Tet-Off™ systems currently available from Clontech (Palo Alto, Calif.). Tet-Off™ system uses the tetracycline-controlled transactivator (tTA), which is composed of the tet repressor protein (TetR) and the VP16 activation domain. tTA activates transcription in the absence of tetracycline. The Tet-On™ system uses the reverse tetracycline-controlled transactivator (rtTA) and activates transcription in the presence of tetracycline. Both systems use the tetracycline-response element (TRE), which contains 7 repeats of the tet operator sequence, and the target gene, such as CXCL5 without any polymorphic sites. tTA or rtTA bind to the TRE, activating transcription of the target gene. This promoter system allows the regulated expression of the transgene controlled by tetracycline or tetracycline derivatives, such as doxycycline. This system could be used to control the expression of ENA-78 in this instant invention.

Other regulatable promoter systems are described in the U.S. patent applications Ser. No. 09/586,625 and provisional application, number to be assigned, filed Jul. 18, 2000, as application Ser. No. 09/619,063 for “Regulation of Gene Expression Using Single-Chain, Monomeric, Ligand Dependent Polypeptide Switches” and subject to a petition for conversion to provisional application, filed Jul. 18, 2001.

The expression vehicles, such as adenoviral vectors or retroviral vectors (including lentiviral vectors), or cells transduced with such expression vehicles, may be employed in the treatment of an inflammatory disease in accordance with the present invention.

The present invention also provides inhibitors (such as atorvastatin and other HMG-coA reductase inhibitors) of ENA-78 for use in the treatment and/or prevention of inflammatory diseases associated with elevated ENA-78 concentrations in a patient. Such inhibitors can be obtained from any number of sources. One preferred source includes various modified version of ENA-78. Such modified versions include ENA-78 having substitutions, additions, and/or deletions of amino acids such that the modified polypeptide loses its ability to activate and attract neutrophils and in fact becomes an inhibitor of ENA-78. Substitutions, deletions, additions that occur in the region of the “active site” of ENA-78 are particularly suitable candidates for inhibitors.

The inhibitors of ENA-78 are useful for treating inflammatory disease characterized by the presence of high numbers of neutrophils. Administration of the inhibitors of ENA-78 of the present invention involves administration of an appropriate amount of a pharmaceutical composition containing the inhibitors as an active ingredient.

In addition to the active ingredient, the pharmaceutical composition may also include appropriate buffers, diluents and additives. Appropriate buffers include, among others, Tris-HCl, acetate, glycine and phosphate, preferably phosphate at pH 6.5 to 7.5. Appropriate diluents include, among others, sterile aqueous solutions adjusted to isotonicity with NaCl, lactose or mannitol, preferably NaCl. Appropriate additives include among others, albumin or gelatin to prevent adsorption to surfaces, detergents (e.g., Tween 20, Tween 80, Pluronic F68), solubilizing agents (e.g., glycerol, polyethylene glycol), antioxidants (e.g., ascorbic acid, sodium metabisulfite) and preservatives (e.g., Thimersol, benzyl alcohol, parabens). A preferred additive is Tween 80.

Administration may be by any conventional means including intravenously, subcutaneously, intramuscularly, or orally. The preferred route of administration is oral administration. Administration may be a single dose or may occur in an appropriate number of divided doses. In one embodiment, atorvastatin is administered orally in any of the commercially available strengths, preferably from 10 mg up to 80 mg. Other potential drugs that could be administered alone or in combination with atorvastatin to treat abnormal ENA-78 include, but are not limited to, ACE inhibitors, angiotensin II receptor antagonists, diuretics, alpha-adrenoreceptor antagonists, cardiac glycosides, phosphodiesterase inhibitors, beta1-adrenoreceptor antagonists, beta-2 adrenoreceptor agonists, leukotriene receptor antagonists, calcium channel blockers, HMG-CoA reductase inhibitors, bile acid sequestrants, fibric acid derivatives, thiazolidinediones, peroxisome proliferator-activated receptor agonists and antagonists, biguanides, imidazoline receptor blockers, endothelin receptor blockers, CETP inhibitors, non-steroidal anti-inflammatory agents (e.g., aspirin, COX-2 inhibitors, ibuprofen and the like), immunologics, and organic nitrites.

Preferably, the pharmaceutical preparation is in unit dosage form. In such form, the preparation is subdivided into unit doses containing the appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small increments until the optimum effect under the circumstances is reached. For convenience, the total daily dosage may be divided and administered essentially continuously or in portions during the day if desired. The amount and frequency of administration will be regulated according to the judgment of the attending clinician considering such factors as age, condition and size of the patient as well as severity of the disease being treated.

Following are examples that illustrate procedures for practicing the invention. These examples should not be construed as limiting. All percentages are by weight and all solvent mixture proportions are by volume unless otherwise noted.

### EXAMPLE 1

### Relation of CXCL5 Gene Polymorphism to Epithelial Neutrophil-Activating Peptide (ENA-78)

Materials and Methods

Study Population

Eligible participants were recruited from the greater Denver and North Central Florida communities and had to be at least 18 years of age without known coronary disease, symptomatic carotid artery disease, peripheral arterial disease, abdominal aortic aneurysm, history of revascularization, or diabetes (i.e., coronary disease or risk equivalents). Other exclusions were pregnancy, malignancy, active alcohol abuse, and use of medications known to affect leukocyte counts (e.g., oral glucocorticoids). Blood samples were obtained from participants enrolled in clinical studies approved by the University of Florida and Colorado Multiple Institutional Review Boards.

CXCL5 Genotype Determination

Genomic DNA was isolated from peripheral blood using a standard protocol (QIAamp®, Qiagen, Valenica, Calif.). Genotypes were determined by polymerase chain reaction (PCR) and pyrosequencing (Langaee T, Ronaghi M, “Genetic variation analyses by Pyrosequencing,” Mutat Res, 573:96-102 (2005)). The PCR reaction mixture and conditions have been previously described (Zineh I et al., “Development and cross-validation of sequencing-based assays for genotyping common polymorphism of the CXCL5 gene,” Clin Chim Acta (Epub, 2006, Mar. 27). Polymorphisms were detected using either the PSQ HS 96A or PSQ 96MA genotyping platforms (Biotage A B, Uppsala, Sweden) by previously developed and validated assays (Zineh supra).

ENA-78 Protein Measurement

ENA-78 protein concentrations were measured in plasma and leukocyte-conditioned media using standard ELISA methods (R&D Systems Inc., Minneapolis, Minn.). Samples were drawn between 7:00 am and 10:00 am to avoid diurnal variation in cytokine expression. For measurement in conditioned media, patients' buffy coat samples were separated from whole blood and immediately transferred to 15 ml conical tubes. Red blood cell lysis solution (Gentra Systems Inc., Minneapolis, Minn.) was added, and samples were mixed and incubated at room temperature for 10 minutes. Samples were then centrifuged at 2000 rpm for 5 minutes. Supernates were removed, and the remaining pellet was resuspended with supplemented RPMI 1640 medium (Mediatech Inc., Herndon, Va.). Cells were incubated in 5% CO₂ atmosphere at 37° C. for 24 hours. They were then harvested and stored, while cell-free media were collected and stored at −80° C. until ELISA was performed. The concentration of ENA-78 for each conditioned medium sample was normalized against mg of total protein using a standard BCA protein assay (Pierce, Rockford, Ill.). All samples were assayed in duplicate.

In Silica Prediction of SNP Functional Effects

The CXCL5 gene sequence was obtained from the University of California Santa Cruz Genome Browser. The gene was run through the Polymorphism Mining and Annotation Programs (PolyMAPr), which screen public SNP databases for reported polymorphisms and interdatabase redundancy (Freimuth R R et al., “PolyMAPr: programs for polymorphism database mining, annotation, and functional analysis,” Hum Mutat, 25:110-7 (2005)). Additionally, PolyMAPr performs functional analyses using JASPAR to screen for potential binding to transcription factor binding sites, PolyPhen which predicts the consequence of non-synonymous SNPs on protein function, the Alternative Splicing Database (ASD) which screens polymorphisms located at splice sites to determine whether they are associated with alternative splicing, and ESE Finder which searches for exonic splicing enhancer elements that could alter mRNA splicing (Sandelin A et al., “JASPAR: an open-access database for eukaryotic transcription factor binding profiles,” Nucleic Acids Res, 32:D91-4 (2004); Ramensky V et al., “Human non-synonymous SNPs: server and survey,” Nucleic Acids Res, 30:3894-900 (2002); Thanaraj T A et al., “ASD: the Alternative Splicing Database,” Nucleic Acids Res, 32:D64-9 (2004); and Cartegni L et al., “ESEfinder: A web resource to identify exonic splicing enhancers,” Nucleic Acids Res, 31:3568-71 (2003)).

Statistical Analyses

Genotype frequencies were determined by allele counting, and observed versus expected genotype frequencies (i.e., HardyeWeinberg equilibrium) were compared by c2 analyses with 1 degree of freedom. Differences in plasma ENA-78 concentrations by genotype were compared by Manne Whitney U test. Because variant homozygosity was expected to occur infrequently for both SNPs, it was decided a priori that −156C/C and 398A/A homozygotes would be grouped with heterozygotes for all analyses, and these groups would be classified as −156C carriers and 398A carriers, respectively. Haplotypes were computationally constructed and pairwise linkage (D0) derived using PHASE software (Ver 2.0.2) (Stephens M et al., “A new statistical method for haplotype reconstruction from population data,” Am J Hum Genet, 68:978-89 (2001)).

Linear regression was performed to determine the joint effects of genetic and non-genetic factors on plasma ENA-78 concentrations. Variables included in the regression model were: age, race (self-reported), sex, body mass index (BMI), white blood cell (WBC) count, smoking status, blood pressure, lipids, and CXCL5 genotypes (coded as zero (0) for homozygous common and one (1) for variant allele carrier). Spearman correlation coefficients were determined to establish the relationships between plasma ENA-78, leukocyte-elaborated ENA-78, and WBC count. Because cell culture assays were performed in a smaller subset of individuals, a test for trend was performed to assess differences in genotype distributions with increasing quartiles of leukocyte-produced ENA-78 concentrations. All statistical analyses were performed using SPSS (version 11.5, SPSS Inc., Chicago, Ill.).

Results

Study Population

Baseline demographics and genotype distributions for the studied population (N=114) are shown in Table 3. The population consisted of relatively young (39±12 years), white individuals with average blood pressure and cholesterol levels. Women represented over 60% of the sample. Overall frequencies of the −156C and 398A variant alleles were 17% and 16%, respectively. The −156C variant allele frequencies were 16%, 4.5%, and 36%, and the 398A variant allele frequencies were 15%, 4.5%, and 36% among white, Latino-Hispanic, and black individuals, respectively.

Genotype distributions for both polymorphisms were in Hardy-Weinberg equilibrium. There was a high degree of linkage (D′=1, r²=0.94) between the two SNPs. All individuals that were wild-type homozygotes for the promoter SNP (i.e., −156G/G) were also wild-type homozygotes at the exon 2 SNP (i.e., 398G/G). Furthermore, all individuals that were variant homozygotes for the promoter SNP (N=2) were also variant homozygotes at the exon 2 SNP. Ninety-four percent of −156G/C heterozygotes were also heterozygotes at the exon 2 position (398G/A). The linkage between the two SNPs was observed regardless of ethnicity.

TABLE 3
Baseline Characteristics

| | Population Value |
|---|---|
| Characteristic | (N = 114) |
| Age (y) | 39 ± 12 |
| Women (%) | 62 |
| White (%) | 76 |
| Hispanic (%) | 10 |
| Black (%) | 6 |
| Current smokers (%) | 18 |
| BMI (kg/m²) | 29 ± 6 |
| SBP (mmHg) | 124 ± 15 |
| DBP (mmHg) | 74 ± 10 |
| TC (mmol/l) | 5.2 ± 1.1 |
| LDL (mmol/l) | 3 ± 0.9 |
| HDL (mmol/l) | 1.5 ± 0.4 |
| TGs (mmol/l) | 1.5 ± 1.4 |
| WBC (×10⁹/l) | 6.3 ± 2 |
| Platelets (×10⁹/l) | 269 ± 54 |
| Genotype (%) | |
| −156 G → C | |
| G/G | 78 (68.4) |
| G/C | 34 (29.8) |
| C/C | 2 (1.8) |
| Genotype (%) | |
| 398 G → A | |
| G/G | 80 (70.2) |
| G/A | 32 (28.1) |
| A/A | 2 (1.8) |
| Allele frequency | |
| −156C | 0.17 |
| 398A | 0.16 |

The data presented in Table 3 as ‘±’ refers to standard deviation (SD). The definitions for certain terms presented in Table 3 are as follows: ‘BMI’ refers to body mass index; ‘DBP’ refers to diastolic blood pressure; ‘HDL’ refers to high-density lipoprotein; ‘LDL’ refers to low-density lipoprotein; ‘SBP’ refers to systolic blood pressure; ‘TC’ refers to total cholesterol; ‘TG’ refers to triglycerides; and ‘WBC’ refers to white blood cell.

Plasma ENA-78 Concentrations by CXCL5 Genotype

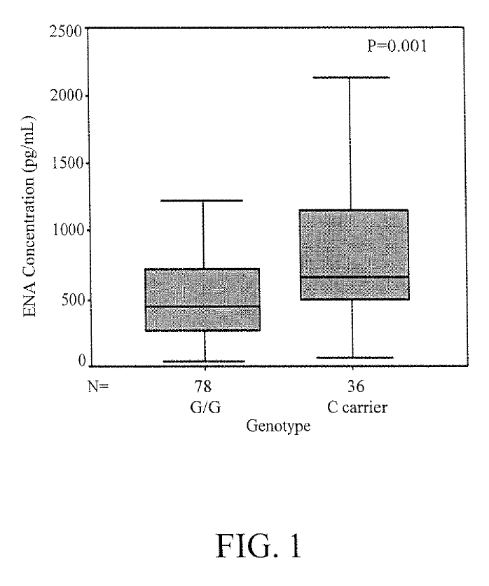

Because of the high degree of linkage between SNPs, plasma ENA-78 concentrations were analyzed by −156G/C promoter genotypes (FIG. 1). Carriers of the variant −156C allele had significantly higher median plasma ENA-78 concentrations (658 pg/ml; IQR: 656 pg/ml) than those with the −156G/G genotype (449 pg/ml; IQR: 452 pg/ml; P=0.001). When including the pre-specified variables of age, race, sex, BMI, WBC, smoking status, blood pressure, lipids, and −156G/C genotype in stepwise regression, the model of best fit was a three-variable model consisting of −156G/C genotype, WBC count, and BMI (Table 4). The model accounted for 16.4% of the variability in plasma ENA-78 concentrations (P<0.001). Furthermore, −156G/C genotype was the variable most significantly associated with plasma ENA-78 concentrations (P=0.004).

TABLE 4
Significant Predictors of Plasma ENA-78 Concentrations

| Variable | β coefficient | Standard error | P-value |
|---|---|---|---|
| Constant | 641 | 255 | 0.013 |
| −156C carrier status | 314 | 106 | 0.004 |
| WBC | 69.8 | 26.8 | 0.011 |
| BMI | −17.3 | 7.7 | 0.027 |

r² = 16.4%,
P < 0.0001;
BMI, body mass index;
WBC, white blood cell.

ENA-78 Production from Cultured Leukocytes by CXCL5 Genotype

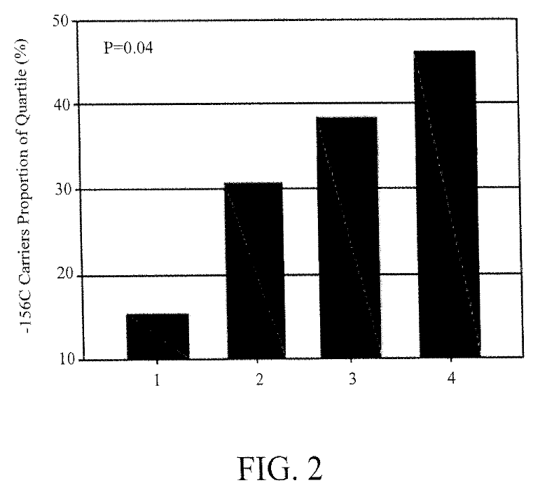

Samples were obtained and leukocytes cultured for a subset of 52 patients from the study population. Correlation coefficients for plasma ENA-78 concentrations, ENA-78 from leukocyte media, and WBC counts are shown in Table 5. There was no significant relationship between plasma and cell culture ENA-78 concentrations. Plasma ENA-78 concentration, but not leukocyte-elaborated ENA-78, was significantly correlated with WBC count (r=0.21, P=0.03). ENA-78 production from leukocytes was also related to the −156G/C polymorphism (FIG. 2). Specifically, there was an incremental increase in number of −156C carriers with increasing ENA-78 quartiles (P=0.04 for trend). Cut-offs for the 25th, 50th, and 75th percentiles were 9 pg/mg, 52 pg/mg, and 179 pg/mg, respectively. The bars in FIG. 2 represent the prevalence of −156C carriers in each quartile.

TABLE 5
Spearman correlation coefficients (r) among WBC, plasma ENA-78,
and leukocyte-elaborated ENA-78 concentrations

| | | Plasma ENA-78 | Leukocyte ENA-78 |
|---|---|---|---|
| | WBC | Concentration | concentration |
| WBC | 1.0 | 0.21^{a} | −0.01 |
| Plasma ENA-78 | | 1.0 | 0.22 |
| Concentration | | | |
| Leukocyte ENA-78 | | | 1.0 |
| Concentration | | | |

WBC, white blood cell;
^{a}P = 0.026.

In Silico Functional Analyses

Both polymorphisms −156G/C and 389G/A were predicted to have functional consequences by PolyMAPr analysis. The −156 promoter polymorphism was found to occur at a transcription factor binding site for myeloid zinc finger proteins (MZF) 1-4. The 398 exonic polymorphism was found to be located in an ESE site.

Discussion

While CXCL5 represents a potential candidate gene for many disease-gene and pharmacogenetic studies, no investigations have assessed whether commonly occurring polymorphisms in this gene have any relationship to ENA-78 protein concentrations. The findings in this study confirm that the −156G/C SNP is highly linked with the 398G/A SNP. This study also confirmed that variant alleles for both SNPs studied occur with relatively high frequency (16-17%) in a population of predominantly European decent. Interestingly, both the −156C and 398A variants occurred with the highest frequency among the small number of African-American patients studied and with the lowest frequency among Latino-Hispanics, compared with their white counterparts. Although African-American and Hispanic groups represented a small proportion of the sample, this variability in allele frequency distributions by racial or ethnic groups is consistent with previous observations for many genes (Marroni A S et al., “Consistent interethnic differences in the distribution of clinically relevant endothelial nitric oxide synthase genetic polymorphisms,” Nitric Oxide, 12:177-82 (2005); Maxwell T J et al., “Beta-2 adrenergic receptor genotypes and haplotypes in different ethnic groups,” Int J Mol Med, 16:573-80 (2005); Roy J N et al., “CYP3A5 genetic polymorphisms in different ethnic populations,” Drug Metab Dispos, 33:884-7 (2005); and Ozawa S et al., “Ethnic differences in genetic polymorphisms of CYP2D6, CYP2C19, CYP3As and MDR1/ABCB1,” Drug Metab Pharmacokinet, 19:83-95 (2004)).

More importantly, this study demonstrated that carriers of the −156C variant allele have higher systemically circulating ENA-78 levels than wild-type homozygotes. In fact, CXCL5 genotype was the most significant predictor of plasma ENA-78 concentrations in multivariable analyses. In order to maximize the robustness of a genetic association with plasma ENA-78 levels if one existed, confounding of cytokine levels was minimized by drawing plasma samples within a narrow time interval in the morning for all patients, while excluding individuals in which acute or chronic diseases could affect ENA-78 concentrations.

Furthermore, in the statistical analysis conducted in this study, other potential confounders were controlled such as smoking status, BMI, lipid levels, and blood pressure. In addition to the significant association between genotype and plasma ENA-78 concentrations, a relationship between the variant allele and leukocyte-produced ENA-78 was also established. There was a significantly increased prevalence of the −156C allele with increasing quartiles of ENA-78 produced from leukocytes in the patients. While CXCL5 genotype was associated with both ENA-78 in the circulation and produced from leukocytes, it was observed that plasma and leukocyte-produced ENA-78 were not correlated with one another. This is perhaps because circulating ENA-78 levels also reflect extra-leukocytic production of this cytokine from such cells as endothelial and vascular smooth muscle cells. Nonetheless, this is the first study to demonstrate the described associations.

To date, no in vitro functional studies have been carried out to elucidate the consequences of these polymorphisms on ENA-78 protein function. In this study, in silico analyses were performed to determine whether the studied SNPs occur at loci that can alter CXCL5 gene transcription in a biologically plausible way. The −156G/C SNP was predicted to bind to a transcription factor binding site for MZF 1-4, and the 398G/A SNP was predicted to occur in an ESE region, both regions important for transcriptional control (Langaee T Y, Zineh I, “Applied molecular and cellular biology,” in Allen W L et al, editors. Pharmacogenomics: applications to patient care. Kansas City, Mo.: American College of Clinical Pharmacy; 2004. p. 53e116).

In fact, the MZF family of proteins is hypothesized to be important in myeloid cell differentiation to monocytes, macrophages, and neutrophilsdall crucial cellular mediators of immunological processes contributing to pathogenesis of leukemia, atherosclerosis, obstructive lung disease, and others (Lenny N et al., “Transcriptional regulation during myelopoiesis,” Mol Biol Rep, 24:157-68 (1997); Hromas R et al., “Hematopoietic transcriptional regulation by the myeloid zinc finger gene, MZF-1,” Curr Top Microbiol Immunol, 211:159-64 (1996); and Nagamura-Inoue T et al., “Transcription factors that regulate growth and differentiation of myeloid cells,” Int Rev Immunol, 20:83-105 (2001)).

ESEs are important in RNA splicing (Zheng Z M., “Regulation of alternative RNA splicing by exon definition and exon sequences in viral and mammalian gene expression,” J Biomed Sci, 11:278-94 (2004)). Findings from the in silico analyses of this study offer mechanistic insights into the observed association between CXCL5 genotype and ENA-78 concentrations.

It is becoming increasingly recognized that systemic, vascular, and cellular inflammation underlie many chronic diseases. Furthermore, neutrophils are key cellular mediators of various adverse pro-inflammatory processes that contribute to disease. In this regard, it becomes important to elucidate the genetic contribution to inflammatory phenotypes in order to extend an understanding of the molecular basis for disease (Lazarus R et al., “Single nucleotide polymorphisms in innate immunity genes: abundant variation and potential role in complex human disease,” Immunol Rev, 190:9-25 (2002)). In addition, recognizing an “at risk” genotype or phenotype aids in identifying individuals who are candidates for more vigilant monitoring for their respective diseases.

Finally, it has been suggested that certain cardiovascular, endocrine, rheumatological, and antineoplastic drugs confer part of their clinical benefit through ENA-78 modulation or interference with neutrophil activity (Kasama T et al., “Neutrophil-derived cytokines: potential therapeutic targets in inflammation,” Curr Drug Targets Inflamm Allergy, 4:273-9 (2005); Zineh I et al., “Modulatory effects of atorvastatin on endothelial cell-derived chemokines, cytokines, and angiogenic factors,” Pharmacotherapy, 26(3):333-40 (2006); Cuzzocrea S et al., “Rosiglitazone, a ligand of the peroxisome proliferator-activated receptorgamma, reduces acute inflammation,” Eur J Pharmacol, 483:79-93 (2004); and Birrell M A et al., “PPAR-gamma agonists as therapy for diseases involving airway neutrophilia,” Eur Respir J, 24:18-23 (2004)). As such, genetic variability in CXCL5 represents a potential contributor to observed variability in drug responses and disease risk.

### EXAMPLE 2

### Genotyping Assays for CXCL5 Polymorphisms

To facilitate future disease-gene and pharmacogenetic investigation of ENA-78, medium- to high-throughput genotyping assays for two commonly occurring CXCL5 polymorphisms (rs352046 and rs425535) were developed and cross-validated in accordance with the subject invention. In this study, allele and genotype frequencies in a U.S. population were compared with those of a previously studied European population. As discussed in more detail below, the results of this study reveal that there is a 100% genotype concordance between the two methods used (Pyrosequencing® and TaqMan®). Variant allele frequencies for rs352046 were consistent between the U.S. (16%) and European (16%) populations, while the rs425535 variant allele was more than twice as high in the European cohort (38% vs. 16%). There was complete linkage of genotypes at both loci in the studied population.

Materials and Methods

Allele frequencies were quantified for 60 consecutively enrolled healthy individuals, and concordance of genotypes was compared between systems. The population was comprised of whites (including two individuals of Middle Eastern ancestry) (86.7%), seven Latino-Hispanics (11.7%), and one person of Asian ancestry (1.7%). There were 23 (38%) and 37 (62%) men and women, respectively.

Genomic DNA was isolated from peripheral blood using a standard protocol (QIAamp®, Qiagen, Valenica, Calif.). Blood samples were obtained from participants enrolled in a clinical study of healthy volunteers approved by the University of Florida Institutional Review Board.

Genotypes were determined by polymerase chain reaction (PCR) with subsequent pyrosequencing or fluorescence-labeled detection methods. The pyrosequencing PCR reaction contained approximately 50 ng of template DNA, 10 pmol each of forward and reverse primers, 1.5 μl dimethyl sulfoxide, 7 μl H2O, and 12.5 μl HotStarTaq™ Master Mix (Qiagen, Valencia, Calif.).

For the −156G→C promoter polymorphism a biotinylated forward 5′-GCGGAGCAGGGTTACAACG-3′ (SEQ ID NO:1) and non-biotinylated reverse 5′-TGGGGCAGTGTGGAAAGAA-3′ (SEQ ID NO:2) primer were used to amplify a 121 bp amplicon containing the polymorphism of interest. For the 398G→A polymorphism in exon 2, a non-biotinylated forward 5′-AGCTGCGTTGCGTTTGTTTAC-3′ (SEQ ID NO:3) and a biotinylated reverse 5′-GCGAACACTTGCAGATTACTGAT-3′ (SEQ ID NO:4) primer were used to amplify a 73 bp amplicon.

PCR conditions were established as follows: 95° C. for 15 min, followed by 45 cycles of 95° C. for 30 s, 58° C. for 30 s (45 s for exon 2), and 72° C. for 30 seconds, and a final extension at 72° C. for 7 minutes. Polymorphisms were detected by pyrosequencing using the PSQ 96MA genotyping platform. Between 4 and 7 μl of PCR product was used for sequencing reactions. Five picomoles of reverse 5′-AGACAATGGGAACTGG-3′ (SEQ ID NO:5) and forward 5′-GTTTACAGACCACGCA-3′ (SEQ ID NO:6) sequencing primers was used in the −156G→C and 398G→A assays, respectively. Genotypes were also determined per TaqMan® protocol (assay IDs C_{—}26260418_{—}10 and C_{—}1015616_{—}10).

Results and Discussion

Overall allele frequencies and comparison of genotype distributions are presented in Table 6. Genotype distributions for both polymorphisms were in Hardy-Weinberg equilibrium by χ2 analysis. Frequencies of both the −156C and 398A variant alleles were 16% in the current sample. There was 100% concordance between the two methods for both the −156G→C and 398G→A polymorphisms. −156C allele frequency was identical to previous reports in a European population (Amoli M M et al., “Two polymorphisms in the epithelial cell-derived neutrophil-activating peptide (ENA-78) gene,” Dis Markers, 21:75-7 (2005)).

However, the 398A allele frequency was previously noted (by Amoli M M et al., “Two polymorphisms in the epithelial cell-derived neutrophil-activating peptide (ENA-78) gene,” Dis Markers, 21:75-7 (2005)) to be 38% in individuals from the U.K., which is more than twice as high as in the present investigation. Furthermore, genotypes were in complete linkage in the current sample, which has not been previously reported.

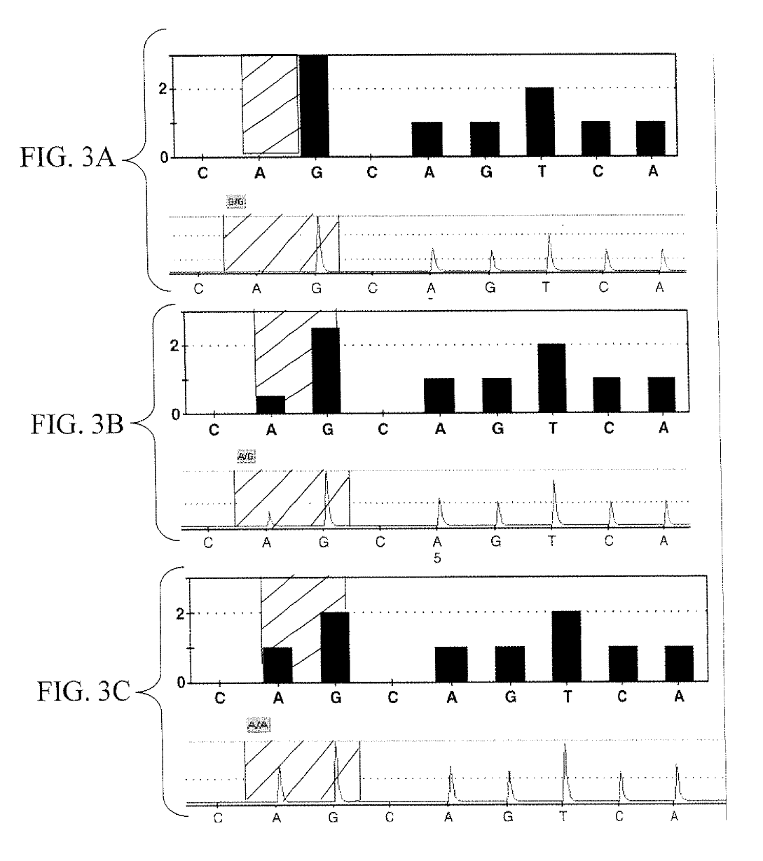

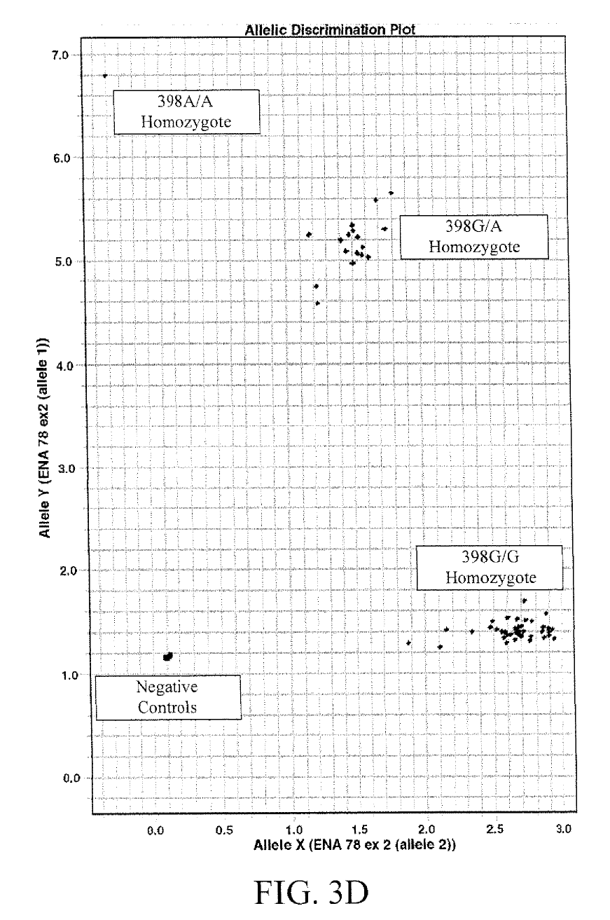

FIG. 3 represents genotype outputs for the 398G→A polymorphism as determined by Pyrosequencing® and TaqMan®. Visual outputs were similar for the promoter polymorphism. Negative controls were confirmed for both SNPs using either platform.

TABLE 6
Genotype discributions and comparisons with previous reports

| Genotype^{b} | Pyrosequencing ®/TaqMan ® | Previously reported^{c} |
|---|---|---|
| −156G→ C | N = 60 | N = 161 |
| G/G | 42 (70%) | 113 (70%) |
| G/C | 17 (28%) | 43 (27%) |
| C/C | 1 (2%) | 5 (3%) |
| Allele | | |
| G | 84% | 84% |
| C | 16% | 16% |
| 398G→ A | N = 60 | N = 63 |
| G/G | 42 (70%) | 20 (32%) |
| G/A | 17 (28%) | 38 (60%) |
| A/A | 1 (2%) | 5 (8%) |
| Allele | | |
| G | 84% | 62% |
| A | 16% | 38% |

^{b}Genotypes were 100% concordant between methods
^{c}From Amoli AA et al., “Interleukin 8 gene polymorphism is associated with risk of nephritis in cutaneous vasulitis,” J Rheumatol, 29: 2367-70 (2002).

CXCL5 represents a possible candidate gene for many inflammatory diseases and may potentially be important in pharmacogenetic studies. In this study, two medium- to high-throughput methods for genotype determination of two commonly occurring CXCL5 SNPs were developed and validated. Data from this study suggest that either Pyrosequencing® or TaqMan® systems may be used for genotyping rs352046 and rs425535 using the described methods, with a high degree of agreement. Furthermore, data from this study indicate a high level of linkage between the promoter and exon 2 SNP, suggesting that genotyping only one of the SNPs might be sufficient in genetic association studies. The skilled artisan could readily confirm the exact degree of linkage using a larger population.

The −156C variant allele frequency (16%) derived from this study was similar to those described previously in populations of healthy individuals from the U.K. and northern Spain (Amoli M M et al., “Two polymorphisms in the epithelial cell-derived neutrophil-activating peptide (ENA-78) gene,” Dis Markers, 21:75-7 (2005); and Amoli A A et al., “Interleukin 8 gene polymorphism is associated with increased risk of nephritis in cutaneous vasculitis,” J Rheumatol, 29:2367-70 (2002)).

However, the 398A variant allele frequency from this study was 16% compared with 38% in Caucasians from the U.K. (Amoli M M et al., “Two polymorphisms in the epithelial cell-derived neutrophil-activating peptide (ENA-78) gene,” Dis Markers, 21:75-7 (2005)). It is unclear why this difference occurred. The 398A variant allele in this study occurred with similar frequency as that reported in dbSNP Build 124 (rs425535), and suggests that perhaps differences in methodology may be responsible for disparate results; while two sequencing-based methods were used in this study for discrimination of this allele, the methods from this study were different than those of Amoli et al. who used ABI PRISM® primer extension. These data highlight the importance of validation of genotype methods using multiple platforms.

### EXAMPLE 3

### Exploration of CXCL5 Gene Polymorphism in Cardiovascular Events

As shown in Example 1, the CXCL5 −156G/C promoter polymorphism has functional consequences, where the C allele may be detrimental in healthy individuals. Since CXCL5 is a neutrophil activator, this study was conducted to investigate whether this polymorphism is associated with adverse clinical events in the International Verapamil SR-Trandolapril Study (INVEST).

INVEST was a 22,575-patient, international outcomes study of two antihypertensive regimens among hypertensives with stable coronary disease. Genetic samples from 271 cases (experiencing myocardial infarction [MI], stroke, or death) and 813 age-, sex- and race-matched controls were used to perform a nested case-control analysis. Logistic regression was used with demographic, clinical, and genetic variables tested in the model.

Mean age was 70±10 years. Blood pressure (BP) at study entry was 148/84±19/11 mmHg. The study was comprised of 50% women, 60% white, 27% Hispanic, and 13% black subjects. The −156C allele frequency was 17% overall, and 11.5%, 18%, and 42.5%, among the above racial groups. The odds ratio for adverse outcomes for −156C carriers versus G/G was 1.15 (95% CI 0.84-1.58). Higher body mass index and systolic BP, and previous MI, heart failure, and diabetes were associated with worse clinical outcomes. The CXCL5 −156 G/C polymorphism was not associated with clinical outcomes in a population of hypertensive patients with stable CAD.

### EXAMPLE 4

### CXCL5 Gene Polymorphism and C-Reactive Protein (CRP)

CRP is a non-specific biomarker of systemic inflammation that has been correlated with increased risk of future cardiovascular disease and events. Not only does CRP appear to be a surrogate for underlying inflammation but also has intrinsic pro-inflammatory properties including the ability to induce cytokine production, attract monocytes, increase expression of adhesion molecules, and reduce expression of endothelial nitric oxide synthase. Elevated CRP is seen in smokers and people with inflammatory disorders and is considered a risk factor for future adverse consequences.

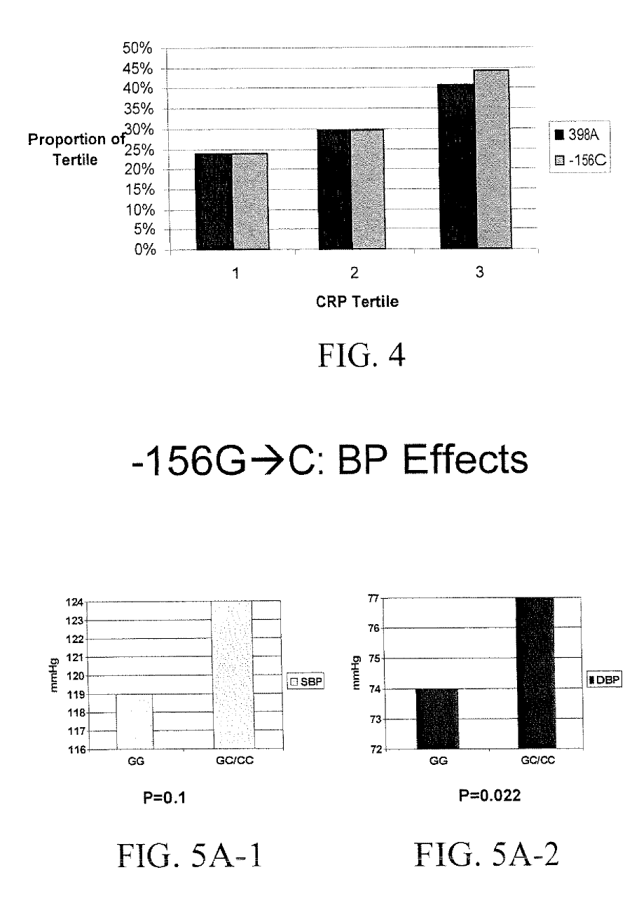

In this Example, the relationship between CXCL5 polymorphisms and CRP concentrations in a current population of cardiovascular disease-free individuals was explored. As demonstrated in FIG. 4, the prevalence of variant carriers of both the −156G→C and 398G→A polymorphisms increases with increasing tertiles of CRP in the population. For example, −156C variant carriers represent approximately 25% of individuals with CRP in the lowest tertile, but 45% of individuals with CRP in the highest tertile. This suggests a relationship between CXCL5 genotypes and a known, validated biomarker/mediator of inflammatory disease risk, such that variant carriers at either locus could be at higher risk for inflammation-related disorders.

### EXAMPLE 5

### CXCL5 Gene Polymorphism and Blood Pressure

Elevated blood pressure is a major risk factor for cardiovascular disease. Furthermore, what was once considered high-normal blood pressure is now considered pre-hypertension. It has recently been shown that elevated white blood cell count is associated with increased systolic blood pressure within the normotensive range (Orakzai et al. J Hum Hypertens 20:341-7 (2006)). Since ENA-78 is an activator of the neutrophil subtype of white blood cells, CXCL5 genotype could be associated with systolic and diastolic blood pressure levels.

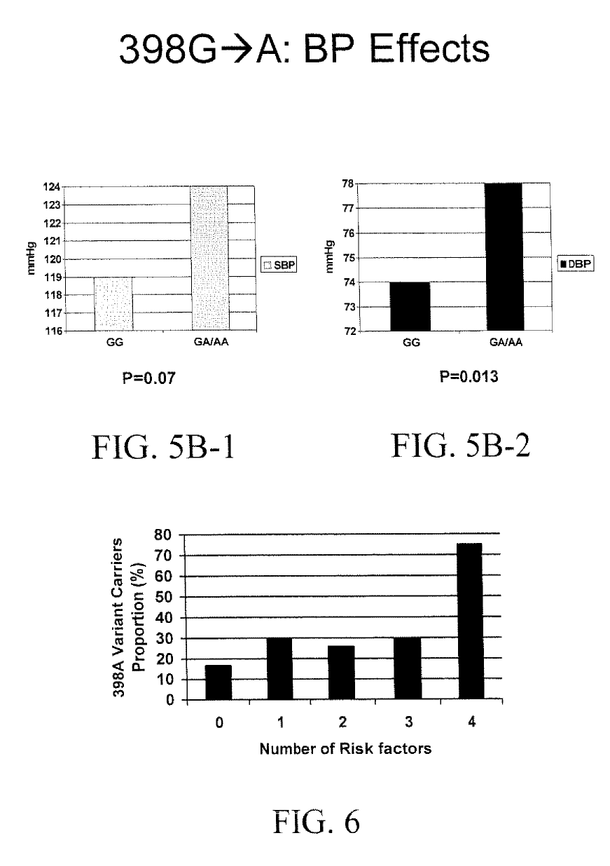

FIGS. 5A1 and 5A2 demonstrate an association between blood pressure and the −156G→C polymorphism such that variant carriers had higher systolic and diastolic blood pressure levels. As demonstrated by FIGS. 5B1 and 5B2, the association is even more significant for the 398G→A polymorphism and blood pressure variables.

To the extent that ENA-78 is important in neutrophil recruitment and degranulation, CXCL5 genotype polymorphisms (−156G>C [rs352046] and 398G>A [rs425535]) was studied to ascertain if one or both polymorphisms could be associated with differences in blood pressure in individuals without established cardiovascular (CVD) disease. Specifically, relatively young individuals without known CVD who were carriers of CXCL5 variant alleles were examined to ascertain if they would exhibit higher systolic (SBP), diastolic (DBP), or pulse pressures than wild-type homozygotes. Furthermore, to assess whether there was a functional role for these polymorphisms, allele-specific mRNA expression of CXCL5 in leukocytes obtained from CVD-free individuals who were heterozygous for the SNPs at both loci was measured.

Methods

Study Population

Participants were recruited from two sites in the United States and had to be at least 18 years of age without known CVD or CVD-risk equivalents as defined by National Cholesterol Education Program criteria. Other exclusions were pregnancy, malignancy, substance abuse, and routine use of medications known to affect WBC counts such as systemic steroids and other anti-inflammatory agents. Individuals were excluded from analysis if they were taking anti-hypertensive medications for either cardiovascular or non-cardiovascular indications (e.g., migraine). For blood pressure measurement, subjects were seated for at least 5 minutes in a quiet GCRC outpatient clinic room and two blood pressure measurements were taken at least 5 minutes apart. The average of the two blood pressure measurements was used for this investigation. Blood samples were obtained from participants enrolled in University of Florida- and Colorado Multiple Institutional Review Board (IRB)-approved studies.

Genotype and Inflammatory Biomarker Determination

Genomic DNA was isolated from whole blood or buccal cells using previously described methods (Andrisin T E, Humma L M, Johnson J A. Collection of genomic DNA by the noninvasive mouthwash method for use in pharmacogenetic studies. Pharmacotherapy. August 2002; 22(8):954-960). CXCL5 genotypes were determined by polymerase chain reaction (PCR) and pyrosequencing (Biotage, Uppsala, Sweden) as described above (see also, Zineh I, Welder G J, Langaee T Y. Development and cross-validation of sequencing-based assays for genotyping common polymorphisms of the CXCL5 gene. Clin Chim Acta. August 2006; 370(1-2):72-75). Plasma high-sensitivity CRP (as a non-specific marker of inflammation) and ENA-78 concentrations were measured by the Shands Hospital laboratory at the University of Florida and by cytometric fluorescence detection as previously described (Luminex™ 100 IS system; Luminex Corp, Austin, Tex.; Fluorokine® MAP Multiplex Human Cytokine Panel A; R&D Systems, Minneapolis, Minn.), respectively (see also, Zineh I, Welder G J, DeBella A E, et al. Atorvastatin effect on circulating and leukocyte-produced CD40 ligand concentrations in people with normal cholesterol levels: a pilot study. Pharmacotherapy. November 2006; 26(11):1572-1577). CRP was measured immediately and plasma samples for ENA-78 determination were stored at −80° C. until cytokine detection was performed.

Allele-Specific mRNA Quantification

To determine whether variant carrier status results in functional changes at the transcriptional level, allele-specific mRNA transcripts from leukocytes were quantified using pyrosequencing-based methodology (see, for example, Sun A, Ge J, Siffert W, et al. Quantification of allele-specific G-protein beta3 subunit mRNA transcripts in different human cells and tissues by Pyrosequencing. Eur J Hum Genet. March 2005; 13(3):361-369; and Shiao Y H, Crawford E B, Anderson L M, et al. Allele-specific germ cell epimutation in the spacer promoter of the 45S ribosomal RNA gene after Cr(III) exposure. Toxicol Appl Pharmacol. Jun. 15 2005; 205(3):290-296). Specifically, presence or absence of allelic expression imbalance was determined using leukocytes obtained from 18 individuals who were heterozygotic for both the −156G>C and 398G>A polymorphisms.

The 398G>A SNP was chosen as the genetic biomarker in these experiments because it is located in the coding region of CXCL5, while −156G>C is a promoter polymorphism and as such cannot be quantified at the mRNA level. Because of the near complete linkage of the studied SNPs, individuals who were heterozygotes at both loci were chosen so that 398G>A genotype might serve as a functional surrogate for the upstream promoter locus.

Leukocyte mRNA was prepared from approximately 6×10⁶ cells from each individual using the RNeasy mini kit (Qiagen, Valencia, USA). Cells were rinsed, lysed, and homogenized in buffered solutions and subsequently passed through the RNeasy mini column (Qiagen, Valencia, USA). Following a series of washes at room temperature and 15 minute incubation with DNase, concentrations were determined by spectrophotometry (NanoDrop Technologies, Wilmington, USA). cDNA was synthesized using approximately 450 ng of cellular RNA from each individual using a High-Capacity cDNA Archive Kit (Applied Biosystems, Foster City, USA) per protocol. Conditions for reverse transcription were 25° C. for 10 minutes followed by 37° C. for 2 hours. cDNA quality was assessed by comparing cDNA and DNA PCR products generated using intron-spanning primers by gel electrophoresis.

For allele-specific transcript quantification, subject DNA and cDNA underwent PCR simultaneously using previously described conditions. PCR products obtained for genotype determination (DNA) and transcript quantification (mRNA) were assayed in parallel pyrosequencing reactions to minimize cycle variability. Pyrosequencing analyses were performed in duplicate on three separate PCR amplification products and the results were pooled for analysis. Peak heights were determined by the pyrosequencing allele quantification algorithm. In genomic DNA, the ratio of 398A:G alleles for DNA in heterozygotes is expected to be approximately 1, whereas significant deviations from this ratio in mRNA would suggest allele expression imbalance associated with the variant allele.

Statistical Analyses

Genotype frequencies were determined by allele counting, and departures from Hardy-Weinberg equilibrium were assessed by chi-square analyses. Differences in blood pressure by genotype groups (0, homozygous for common allele; 1, heterozygous or homozygous for variant allele) were compared using one-way ANOVA. Multivariate analysis was performed if blood pressure differences were seen across genotype groups. Covariates for multivariate analysis were chosen through univariate analyses of age, sex, smoking status (0, non-smoker; 1, current smoker), body mass index (BMI), CRP concentration, ENA-78 concentration, and WBC count. Any variable with a P≦0.1 on univariate analysis was entered into the multivariate model. Because of small numbers of individuals within racial groups, analyses could not be performed within racial strata. However, race (0, white; 1, non-white) was included in all multivariate analyses, and a race-by-genotype interaction term was considered in the regression models to avoid spurious associations secondary to racial differences in allele frequency. Multiple linear regression using step-type selection methods was performed to determine the joint effects of CXCL5 genotypes and clinical variables on SBP, DBP, or pulse pressure. All statistical analyses were performed using SPSS (version 11.5, SPSS Inc., Chicago, Ill.) or SAS (version 9.1, SAS Institute Inc., Cary, N.C.). A p-value <0.05 was considered statistically significant.

Results

Baseline demographic characteristics are shown in Table 7. Participants were on average 39±12 years old with blood pressures of 126/75±15/11. −156G>C and 398G>A genotypes were determined for 189 and 188 of the 192 individuals, respectively. The overall −156C and 398A minor allele frequencies were both 15%. Variant allele frequencies differed by race whereby the −156C allele frequency was 14%, 45%, and 11% and 398A allele frequency was 13%, 46%, and 9% in Caucasians, blacks, and non-black Hispanics, respectively. Genotype distributions satisfied criteria for Hardy-Weinberg equilibrium. The two SNPs were in a high degree of linkage disequilibrium with r² for Caucasian, black, and Hispanic individuals of 0.82, 1.0, and 0.51, respectively in the study population. Based on the preexisting sample size and prevalence of variant alleles, 80% power with a two-sided cc of 0.05 was used to detect a 6 mmHg difference in SBP, 4 mmHg difference in DBP, and 4 mmHg difference in pulse pressure between genotype groups.

TABLE 7
Baseline Characteristics

| | Population Value |
|---|---|
| Characteristic* | (N = 192) |
| Age, mean ± SD, years | 39 ± 12 |
| Women | 124 (65) |
| Race/ethnicity | |
| White | 148 (77) |
| Black | 12 (6) |
| Hispanic | 19 (10) |
| Other | 13 (7) |
| Family heart disease history | 29 (15.1) |
| Smoking | 35 (18) |
| BMI, mean ± SD, kg/m2 | 29.6 ± 7 |
| Blood pressure, mean ± SD, mmHg | |
| Systolic | 126 ± 15 |
| Diastolic | 75 ± 11 |
| Pulse pressure, mean ± SD, mmHg | 51 ± 10 |
| Cholesterol, mean ± SD, mg/dL^{†} | |
| Total | 201 ± 43 |
| LDL | 118 ± 36 |
| HDL | 55 ± 17 |
| Triglycerides | 139 ± 107 |
| White blood cell count, mean ± SD, | 6.3 ± 2.0 |
| ×10⁹ cells/L | |

*Values expressed as number (percentage) unless otherwise indicated.
^{†}Total, HDL, and triglycerides available in 94% of subjects; LDL available in 92% of subjects.

Genotype Association with Blood Pressure

In −156C variant carriers, SBP was 7 mmHg higher than in −156G/G wild-type homozygotes (131±17 vs. 124±14 mmHg; P=0.008). Similarly, DBP was 4 mmHg higher in −156C variant carriers (78±11 vs. 74±11 mmHg; P=0.013). Pulse pressure did not differ between −156C variant carriers and wild-type homozygotes (53±11 vs. 51±10; P=0.22). Because of the high degree of linkage disequilibrium between the 398G>A and −156G>C SNPs, blood pressure differences were similar when compared by 398G>A genotypes. For example, SBP was 130±16 and 125±14 mmHg in 398A variant carriers and 398G/G homozygotes, respectively (P=0.033); DBP was 78±11 and 74±11 mmHg, respectively (P=0.038); and PP was not different between groups (53±11 vs. 51±10 mmHg in 398A carriers and 398G/G homozygotes, respectively; P=0.362).

Age (P≦0.001), sex (P≦0.008), and BMI (P≦0.002) were common univariate predictors of SBP, DBP, and pulse pressure. Furthermore, WBC count (P=0.10 for SBP; P=0.076 for DBP) and both CXCL5 polymorphisms (range P=0.008 to 0.038) were additional predictors of SBP and DBP, while smoking status was associated with SBP alone (P=0.038). In terms of circulating CRP and ENA-78 levels, both biomarkers were significant for SBP (P=0.005 for CRP and P=0.033 for ENA-78) and PP (P=0.001 for CRP and P=0.007 for ENA-78) in univariate analyses. Consistent with the results provided in Example 1, FIG. 1, CXCL5 genotype was associated with ENA-78 protein concentrations in the plasma whereby variant carriers at either SNP locus had higher protein concentrations than wild-type homozygotes.

In multivariate analysis with SBP, age, sex, BMI, and the CXCL5 −156G>C promoter polymorphism were identified as significant variables (Table 8). The overall model that included these variables explained 32.5% of the variability in SBP (P<0.001). Consideration of the 398G>A polymorphism rather than the −156G>C promoter SNP resulted in a model in which only age, sex, and BMI were significantly associated with SBP (R²=0.301; P<0.001).

TABLE 8
Multivariate Predictors of SBP in CVD-Free Individuals

| Variable | β | Standard error | p-value |
|---|---|---|---|
| Constant | 100 | 4.86 | <0.0001 |
| Age | 0.313 | 0.094 | 0.001 |
| Sex | −9.84 | 2.12 | <0.0001 |
| BMI | 0.637 | 0.160 | <0.0001 |
| −156C carrier | 4.93 | 2.30 | 0.034 |

R² = 0.325;
P < 0.0001.

Age, sex, and the −156G>C SNP were further associated with DBP, along with WBC (Table 9). Consideration of this promoter SNP (model R²=0.168; P<0.0001) was slightly more informative than consideration of the 398G>A SNP (P=0.067) in which case age (P<0.0001), sex (P=0.001), and WBC (P=0.02) still remained significant (model R²=0.145; P<0.0001). In multivariate models of PP, only sex (P<0.004) and BMI (P<0.0001) were significant (model R²=0.247; P<0.0001).

TABLE 9
Multivariate Predictors of DBP in CVD-Free Individuals

| Variable | β | Standard error | P-value |
|---|---|---|---|
| Constant | 63.13 | 3.42 | <0.0001 |
| Age | 0.247 | 0.063 | <0.0001 |
| Sex | −5.801 | 1.549 | <0.0001 |
| −156C carrier | 3.735 | 1.630 | 0.023 |
| WBC | 0.768 | 0.374 | 0.041 |

R² = 0.168;
P < 0.0001.

Allelic Expression Imbalance

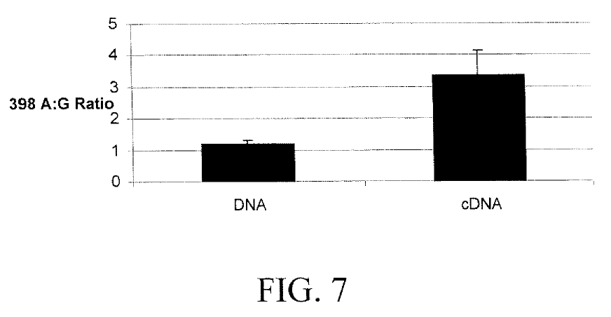

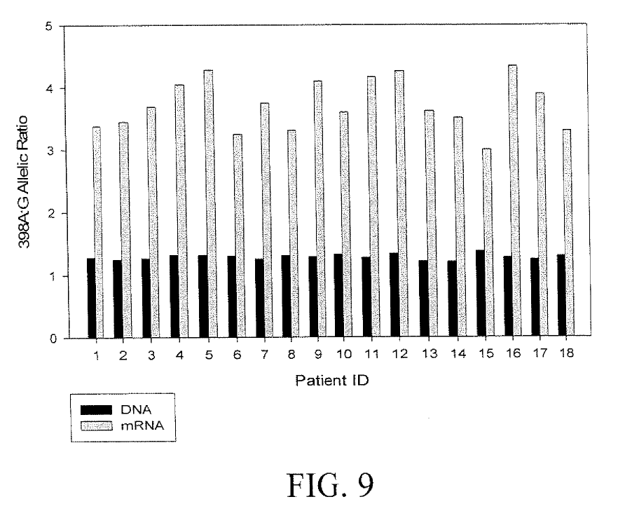

Allele-specific mRNA quantification was performed to determine whether there is a functional basis for the differences seen in blood pressure based on CXCL5 genotypes (see Methods for rationale of 398G>A as marker SNP). Importantly, there was consistently higher expression of CXCL5 mRNA from the 398A allele compared to the 398G allele in heterozygous individuals (FIG. 9). For example, individual heterozygotes displayed anywhere from 2.2-fold to 3.4-fold higher expression of 398A variant transcripts compared to the 398G allele, with a mean ratio of 2.9. This ratio obtained from RNA is significantly different than that obtained when DNA is used where the ratio is 1. Such results are consistent with those described in Example 7 below (see FIG. 7; P=7.4E−15).

Discussion

Accumulating evidence points to a relationship between inflammation and blood pressure. Data suggest that WBC counts are associated with incident hypertension and correlated with blood pressure concentrations. According to the subject invention, WBC count is a surrogate for leukocytic chemokine activity, and the CXCL5 gene, which encodes the neutrophil attractor ENA-78, is an important determinant of blood pressure. As demonstrated herein, a significant, independent relationship exists between CXCL5 polymorphisms and SBP and DBP in the overall population of CVD-free individuals. Variant carriers of the −156G>C promoter SNP had 7 mmHg and 4 mmHg higher SBP and DBP, respectively, than those with the wild-type −156G/G genotype. Because of the epidemiologically significant difference in CVD risk conferred by blood pressure differences of this magnitude, and since variant carriers represent approximately 30% of the population studied, CXCL5 polymorphisms are considered herein as potential novel biomarkers of pre-hypertension, hypertension, and CVD risk.

Of particular interest, WBC count (along with traditional variables such as age, sex, smoking status, and BMI) was significantly associated with SBP and DBP in univariate analysis among CVD-free individuals. This finding supports recent research that demonstrated a relationship between WBC counts and SBP among nearly 3,500 white individuals without CVD and with SBP<140 mmHg on entry (Orakzai R H, Orakzai S H, Nasir K, et al. Association of white blood cell count with systolic blood pressure within the normotensive range. J Hum Hypertens. May 2006; 20(5):341-347). However, in the subject invention, WBC count was no longer a significant predictor of SBP when CXCL5 genotype was included in multivariate analysis, suggesting genotype may capture the contribution of inflammation to SBP more effectively than WBC count. WBC did, however, remain a significant predictor of DBP in multivariate analysis, along with age, sex, and CXCL5 −156G>C genotype.

To determine whether there is any functional basis for an observed association between CXCL5 variant alleles and blood pressure, allele expression imbalance experiments were performed in a subset of participants. The exonic 398G>A allele was chosen as the genetic marker given its location in the coding region of the mRNA. However, the 398G/A heterozygous individuals (N=18) were also heterozygous for the promoter polymorphism, which minimizes confounding of an association by differing genotypes at the upstream locus. It was noted that variant carriers displayed nearly 3-fold higher expression of variant CXCL5 mRNA transcripts from the 398A allele. This novel finding is consistent with observations described herein that variant carriers exhibited higher plasma and leukocyte-produced ENA-78 than wild-type homozygotes, and that the promoter and exonic SNPs occur in transcription factor binding and splicing enhancer sites, respectively. Given that the −156G>C and 398G>A SNPs are in near perfect linkage disequilibrium, it is unclear which polymorphism is the causal variant and functionally contributes to the blood pressure phenotype. However, the −156G>C promoter SNP was more significantly correlated with blood pressure in the present study.

In addition to genotype and traditional covariates, the present analyses included plasma CRP and ENA-78 protein concentrations. While CRP and ENA-78 were significantly associated with SBP (and PP) in univariate analyses, they fell out of the models when CXCL5 genotype was included. This suggests that in the present analyses, genotype is more significantly associated with the blood pressure phenotype than systemically circulating concentrations of the non-specific inflammatory mediator CRP and the CXCL5 protein product ENA-78. While this observation may appear somewhat contradictory, it can be postulated that CXCL5 gene polymorphisms may be better surrogates of chemokine activity at the target organ (e.g., endothelial) level than a measurement in the circulation. Because of trans-acting influences on systemic biomarker expression, polymorphisms in CXCL5 may be more robustly associated with blood pressure.

In general, there is biological plausibility for the role of CXCL5 in CVD. For example, the protein product of CXCL5, ENA-78, belongs to the same class of chemokines as IL-8, IP-10, and I-TAC, which have been previously implicated in atherosclerotic inflammation. ENA-78 has been shown to be chemotactic for neutrophils and stimulate neutrophilic degranulation causing release of myeloperoxidase and generating reactive oxygen species. In addition, ENA-78 is involved in platelet-dependent activation of monocytes, displays angiogenic properties, and has been implicated in ischemic stroke, abdominal aortic aneurysm, and thrombotic states such as anti-phospholipid syndrome. To the extent that hypertension is a risk factor for adverse events such as stroke and abdominal aortic aneurysm, ENA-78 is overexpressed in these situations, and as shown herein, CXCL5 polymorphisms are associated with both ENA-78 concentrations and blood pressure. As final evidence of a link between the CXCL5 pathway and blood pressure, statins have been hypothesized to have mild antihypertensive effects and it has been described herein that atorvastatin reduces ENA-78 production from human endothelial cells in a dose-dependent fashion (see, for example, Zineh I, Luo X, Welder G J, et al. Modulatory effects of atorvastatin on endothelial cell-derived chemokines, cytokines, and angiogenic factors. Pharmacotherapy. March 2006; 26(3):333-340; and Milionis H J, Liberopoulos E N, Achimastos A, et al. Statins: another class of antihypertensive agents? J Hum Hypertens. May 2006; 20(5):320-335).

### EXAMPLE 6

### CXCL5 Gene Polymorphism and Lipoproteins

In addition to blood pressure and CRP, cholesterol levels are major predictors of adverse cardiovascular events. Recently, the ratio between atherogenic apolipoprotein (apo) B and anti-atherogenic apoA1 particles (apoB/apoA1 ratio) has been identified as the most significant predictor of myocardial infarction worldwide (Yusuf S et al. Lancet 364(9438):937-52 (2004)). This association has been attributed to the presence of dyslipidemia among patients in various global regions. In this example, whether CXCL5 genotypes are related with apoB/apoA ratio in a population of relatively healthy, untreated individuals was explored.

It was found that for the −156G→C polymorphism, C variant carriers had higher apoB/apoA ratios compared with those with the −156GG genotype (0.63±0.24 vs. 0.55±17, P=0.09). Furthermore, in a similar analysis conducted by 398G→A genotype, it was found that A variant carriers had a ratio of 0.64±0.24 compared to 0.55±0.17 in 398GG homozygotes (P=0.06).

### EXAMPLE 7

### CXCL5 Gene Polymorphism and Risk Factor Clustering

Examples 4-6 suggest a relationship between CXCL5 gene polymorphisms and traditional risk factors for such conditions as cardiovascular disease, namely systolic and diastolic blood pressure, lipoproteins, and CRP. The magnitude of the genetic association is similar for the −156G→C and 398G-A polymorphisms as these SNPs are highly linked and much of the clinical information would be expected to be captured by genotyping one SNP. In addition to these individual associations, it was attempted in this Example to determine whether there are differences in risk factor clustering and CXCL5 genotypes. This would help elucidate whether these SNPs are associated with individual risk factors or clustering of risk factors that could inform a global risk stratification schema.

In an exploratory analysis of cardiovascular disease-free individuals without dyslipidemia or diabetes, the prevalence of variant alleles (398A) with increasing numbers of risk factors was determined. Specifically, the presence of a risk factor was defined as CRP, SBP, DBP, or apoB/apoA ratio above the population median. The number of risk factors for a given person, therefore, could range from 0 to 4 for these well-validated clinical variables. As shown in FIG. 6, individuals with 1, 2, or 3, risk factors were approximately 1.5 times more likely to be 398A variant carriers than those with 0 risk factors. Those with 4 risk factors were 4.5 times more likely to be 398A variant carriers than those with no risk factors.

### EXAMPLE 8

### Quantification of Allele-Specific CXCL5 mRNA/cDNA Transcripts From Peripheral Circulating Leukocytes

Examples 1-7 support the assertion of the present invention that functional CXCL5 polymorphisms contribute to variable at-risk phenotypes. To further strengthen this association, this Example determined whether or not the 398G→A polymorphism was associated with allele-specific differences in CXCL5 transcription using the cis-trans method. The CXCL5 mRNA was isolated using standard procedures from 18 individuals with the 398G/A heterozygous genotype. Subsequently, RT-PCR was performed to quantify the ratio of A:G mRNA alleles in these individuals with DNA as a control.

The results are shown in FIG. 7. As expected, the A:G ratio in DNA was close to 1. However, this ratio was greater than 3 for mRNA, suggesting increased transcription of the variant in carriers of the variant genotype. Compared with control (DNA), this ratio suggests a 2.8 fold enhanced transcription of the A allele (P<1E−25) in variant carriers, which is consistent with differences in translated ENA-78 protein levels as demonstrated in Example 1.

### EXAMPLE 9

### Identification of Fibrates for Use in Treating Conditions Associated with Abnormal ENA-78 Levels

The peroxisome proliferator-activated receptors (PPARα) agonists, or fibrates, are a class of lipoprotein-modulating drugs commonly used in the treatment of dyslipidemia. By virtue of their ability to increase peripheral lipolysis and decrease hepatic triglyceride production, the fibrates are among the best agents for lowering triglycerides and increasing high-density lipoprotein cholesterol (HDL-C) in mixed dyslipidemias. In addition, intervention trials have shown fibrates to slow atherosclerotic progression and improve cardiovascular outcomes in certain subsets of patients.

It has been hypothesized that the cardioprotective effects of fibrates are in part due to nuclear receptor-mediated anti-inflammatory effects. In fact, fibrates have been shown to blunt inflammatory processes in monocytes and macrophages, T lymphocytes, endothelial cells, vascular smooth muscle cells, and adipocytes, largely through down-regulation of cascades involving inflammatory cytokines. While the majority of studies have investigated modulatory roles of fibrates on adhesion molecules and monocytic chemokines, there is a paucity of data regarding the effect of fibrates on neutrophilic chemokines such as epithelial neutrophil activating protein (ENA-78). ENA-78 is important in the recruitment of neutrophils to sites of endothelial injury and propagating the effects of neutrophil activation. As described herein, ENA-78 has been implicated in ischemic stroke and other cardiovascular-related conditions.

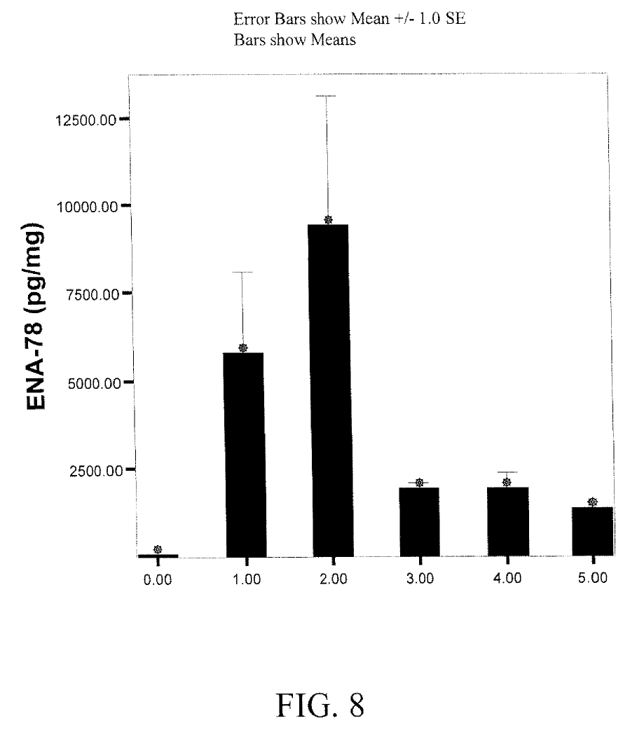

While it has been shown that the cardioprotective drug atorvastatin lowers endothelial production of ENA-78, the data for fibrate effects on ENA-78 are non-existent. As such, the influence of fenofibrate on IL-1β-stimulated production of ENA-78 from human endothelial cells was investigated. FIG. 8 illustrates endothelial ENA-78 production at baseline (0), after IL-1β stimulation (1), and after IL1β stimulation in the presence of fenofibrate ranging from 1 to 50 uM (2-5). What can be seen is a reduction in ENA-78 production at fenofibrate concentrations of 10, 25, and 50 uM (3-5) relative to the other IL-1β-stimulated conditions. As such, fenofibrate and other PPARα agonists appear to modulate endothelial cell production of ENA-78, which make them effective in treating various diseases associated with higher than normal levels of systemically circulating ENA-78 levels.

### EXAMPLE 10

### Identification of Statins for Use in Treating Conditions Associated with Abnormal ENA-78 Levels

Vascular inflammation has been implicated as the predominant catalyst for the onset and development of atherosclerotic lesions. The immunopathology of atherosclerosis begins with adhesion of leukocytes to the surface of the endothelium, followed by their migration across the surface endothelium into subendothelial layers. This migration largely depends on chemotactic cytokines (chemokines). Inflammatory cells then become components of the atherosclerotic lesion through initiation and propagation of the inflammatory cascade, largely by shifting the predominance of local cytokines toward a proinflammatory state. Cellular responses to this proinflammatory condition include angiogenesis and further elevation of inflammatory cytokine concentrations. As such, vascular disease can partly be seen as a complex interaction among the endothelium, chemokines, endogenous proinflammatory and antiinflammatory cytokines, and angiogenic factors.

3-Hydroxy-3-methylglutaryl coenzyme A (HMG-CoA) reductase inhibitors (statins) possess antiinflammatory properties. Although statins individually modulate select inflammatory markers or mediators, no comprehensive characterization of the simultaneous effects of statins on protein concentrations of chemokines, prototypical inflammatory cytokines, antiinflammatory cytokines, or angiogenic factors derived from the vascular endothelium have been made. Furthermore, the dose-related effects of statins on these molecules have not been fully investigated. Finally, whether the immunomodulatory effects of statins are independent of their ability to inhibit HMG-CoA reductase is debated. In accordance with the subject invention, the effect of atorvastatin on the production of chemokines, prototypical proinflammatory and antiinflammatory cytokines, and angiogenic factors (Table 10) from human endothelial cells were investigated to gain insights into its mechanisms. Furthermore, the concentration-dependent effects of atorvastatin on changes in immune mediators were characterized and it was determined whether these are related to inhibition of HMG-CoA reductase. Finally, changes in the biomarkers of interest in a uniform system were simultaneously measured.

TABLE 10
Immunomodulatory Molecules Implicated in Vascular Pathogenesis

| Class | Abbreviations | Function |
|---|---|---|
| Chemokines | | |
| Epithelial neutrophil-activating peptide-78 | ENA-78/CXCL5 | Attracts and activates neutrophils |
| Interleukin 8 | IL-8/CXCL8 | Attracts and activates neutrophils |
| Monocyte chemotactic protein 1 | MCP-1/CCL2 | Attracts and activates monocytes & macrophages |
| Proinflammatory or antiinflammatory cytokines | | |
| Interleukin 6 | IL-6 | Pleiotropic proinflammatory molecule; stimulates adhesion |
| | | molecule and chemokine expression, coagulation, platelet |
| | | activation, and other effects |
| Interleukin 10 | IL-10 | Suppresses proinflammatory Th1-related cytokine production |
| Angiogenic factors | | |
| Fibroblast growth factor | FGF | Stimulates proliferation of fibroblast, endothelial, and smooth |
| | | muscle cells |
| Granulocyte colony-stimulating factor | G-CSF | Stimulates proliferation, differentiation, and activation of |
| | | neutrophilic granulocyte hematopoietic cells |

Methods
Cell Culture and Treatment

Human umbilical vein endothelial cells were obtained (Clonetics Cell Systems; Cambrex Bio Science Walkersville, Inc., Walkersville, Md.). Approximately 5×10⁵ viable cells were seeded in endothelial cell growth-supplemented basal medium (EGM-2-endothelial cell medium-2; Cambrex Bio Science Walkersville, Inc.) and maintained at 37° C. under a 5% carbon dioxide atmosphere per manufacturer's protocol. The culture medium was changed 24 hours after seeding and every 48 hours thereafter until confluence. Cells were then treated with trypsin, seeded in a 48-well culture dish, and incubated under the conditions previously described. Cells used in this Example were from the third passage.

After cells reached 70-80% confluence in the culture dish, they were allowed to become quiescent in unsupplemented, serum-free medium for 24 hours before treatment and then placed in medium containing 2% fetal bovine serum. Atorvastatin (LKT Laboratories Inc., St. Paul, Minn.) was dissolved in dimethylsulfoxide (Sigma-Aldrich, St. Louis, Mo.). Cells were treated for 24 hours with atorvastatin 1-50 μM alone or with atorvastatin plus mevalonate 250 μM (Sigma-Aldrich). Controls were cells cultured without atorvastatin or atorvastatinmevalonate. The final concentration of dimethylsulfoxide in all conditioned media did not exceed 0.1%. All experiments were performed in duplicate.

After treatment, culture-conditioned media were collected, divided into aliquots, and stored at −80° C. for no longer than 7 days until multiplex measurement of chemokines, cytokines, and angiogenic factors was performed.

Multiplex Protein Measurement and Analyses

The culture-conditioned media protein content of epithelial neutrophil-activating peptide-78 (ENA-78), interleukin-8, (IL-8), monocyte chemotactic protein-1 (MCP-1), interleukin-6 (IL-6), interleukin-10 (IL-10), fibroblast growth factor (FGF), and granulocyte colony-stimulating factor (G-CSF) were measured by means of flow-based immunofluorescence multiplex detection (Fluorokine MAP Human Cytokine Panel; R&D Systems Inc., Minneapolis, Minn.) with the Luminex 100 IS platform (Luminex Corp., Austin, Tex.). Concentrations for each sample were normalized against milligrams of total protein by using a standard bicinchoninic acid protein assay (Pierce Biotechnology, Inc., Rockford, Ill.). Samples were assayed in duplicate.

Standard curves of best fit for each analyte were generated by plotting the median fluorescence intensities of the analyte standards against their known concentrations by using Beadview multiplex data analysis software version 1 (Upstate, Charlottesville, Va.).

Differences in biomolecular concentrations were compared by treatment group by applying one-way analysis of variance and a post-hoc Tukey's test as appropriate. The Jonckheere Terpstra test was performed to assess the trend for differences in biomolecular concentrations across concentrations of atorvastatin. A p value below 0.05 was considered to indicate a statistically significant difference. Statistical analyses were performed with SPSS software, version 11.5 (SPSS Inc., Chicago, Ill.).

Results

Concentration-Dependent Effects on Chemokine Production

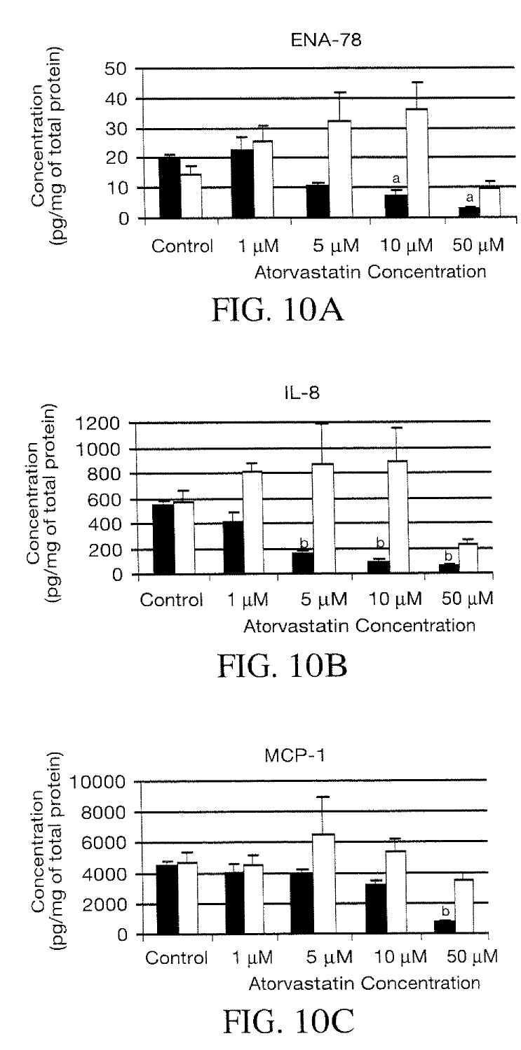

FIG. 10 shows the effect of atorvastatin on endothelial cell production of ENA-78, IL-8, and MCP-1. Under the culture conditions described, human umbilical vein endothelial cells produced ENA-78 at a mean±SE concentration of 19±2 pg/mg protein, which was not significantly altered after treatment with lower concentrations of atorvastatin. However, escalating concentrations of atorvastatin resulted in a progressive 45-85% reduction in ENA-78 production compared with findings in untreated controls (p=0.002 for atorvastatin 10 μM and p.<0.001 for atorvastatin 50 μM vs. control), see FIG. 10A. The effect of atorvastatin on ENA-78 was concentration dependent (p.<0.001 for concentration effect). Cotreatment of endothelial cells with atorvastatin and mevalonate reversed the effect of atorvastatin on ENA-78 production.

Atorvastatin also reduced IL-8 production in a concentration-dependent manner (FIG. 10B). The minimum effective concentration of atorvastatin that resulted in a significant reduction in IL-8 compared with control was 5 μM (155±45 vs. 551±69 pg/mg protein, p.<0.001). The concentration effect of atorvastatin on IL-8 production was significant as concentrations were lowered by 26%, 72%, 83%, and 89% in the four atorvastatin groups (p.<0.001 for concentration effect). The addition of mevalonate to atorvastatin reversed the drug effect in all atorvastatin groups.

FIG. 10C shows the effect of atorvastatin on MCP-1. Significant reduction in MCP-1 (4556±514 vs. 782±105 pg/mg protein, 83% reduction, p.<0.001) was observed with only the highest concentration of atorvastatin, and cotreatment with mevalonate reversed this effect. Although individual MCP-1 concentrations did not significantly differ among concentrations of atorvastatin less than 50 μM and the control condition, the overall concentration effect was significant (i.e., increasing reduction in MCP-1 concentrations with increasing concentrations of atorvastatin, p<0.001).

Concentration-Dependent Effects on Proinflammatory and Antiinflammatory Cytokine Balance and Angiogenic Factors

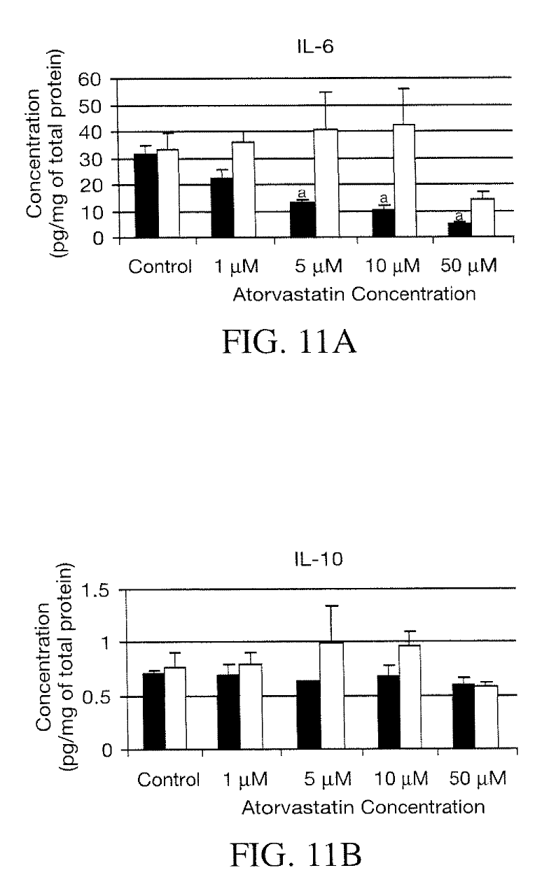

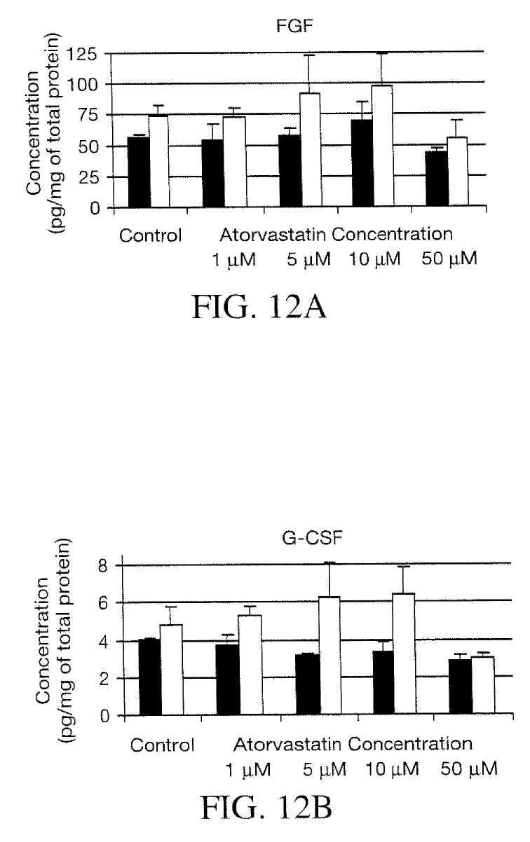

FIGS. 11A and 11B show the effect of atorvastatin on the prototypical proinflammatory cytokine IL-6 and generally antiinflammatory cytokine IL-10. Although a significant, graded reduction of IL-6 production was observed with increasing concentration of atorvastatin (29-83%, p<0.001 for concentration effect), no treatment effect on IL-10 was found. Exposure to mevalonate eliminated the effect of atorvastatin on IL-6. Furthermore, concentrations of FGF or G-CSF did not change when endothelial cells were treated with atorvastatin (FIGS. 12A and 12B).

Discussion

Although statins lower systemic concentrations of nonspecific markers and mediators of inflammation, the exact immunomodulatory effects of these agents on the vascular endothelium are not completely known. The data in this Example suggest that atorvastatin simultaneously lowers chemokine concentrations of the neutrophil attractors IL-8 and ENA-78 and the monocyte attractor MCP-1. Initial treatment effects on IL-8 and ENA-78 were observed with atorvastatin 5-10 μM, and protein concentrations progressively decreased with increasing concentrations of atorvastatin. In addition, MCP-1 was reduced at the highest concentration of atorvastatin.

Data generated in this Example suggest that the effect of atorvastatin against IL-8 occurs at concentrations lower than those generally used in gene expression studies, that these effects culminate at the highest studied concentration, and that IL-8 concentrations are modulated even in non-stimulated endothelial cells. No investigators have studied the effect of any statin on the modulation of ENA-78 protein concentrations. The chemokine ENA-78 is a member of the CXC class that includes IL-8, interferon-inducible protein-10, interferon-inducible T-cell a chemoattractant, and other biomolecules important in cardiovascular pathogenesis. In particular, ENA-78 is responsible for the recruitment and activation of neutrophils. Neutrophilic degranulation and subsequent release of myeloperoxidase leads to the generation of reactive oxygen species, which adversely affects vascular integrity and function. Furthermore, ENA-78 is involved in platelet-mediated monocyte activation and may act as an angiogenic factor detrimental in the development of cardiovascular disease. To the extent that ENA-78 mediates adverse inflammatory processes in the vasculature, the reduction of ENA-78 concentrations by atorvastatin, as suggested herein, represents a novel mechanism for the beneficial effects of statins in cardiovascular and other diseases.

In addition to lowering concentrations of the aforementioned chemokines released from vascular endothelial cells, atorvastatin lowered concentrations of the multifunctional proinflammatory cytokine IL-6, as has been demonstrated in several in vitro models and in vivo. However, these studies largely failed to account for whether statins augment the effects of endogenous antiinflammatory cytokines in addition to lowering concentrations of proinflammatory cytokines. Therefore, it was determined in this Example whether atorvastatin shifts the molecular balance toward an antiinflammatory state at the endothelial level by simultaneously lowering IL-6 and increasing IL-10 concentrations. Under basal conditions, atorvastatin seemed to lower IL-6 production with no appreciable effects on the largely antiinflammatory IL-10. This finding cannot be extrapolated to high-inflammatory states, but it offers insights into the effects of atorvastatin on the proinflammatory-antiinflammatory balance.

No drug effect on concentrations of FGF or G-CSF was observed. Fibroblast growth factor stimulates the proliferation of a variety of cells, including fibroblasts and endothelial cells, implicated in vascular health and disease. Because FGF is involved in processes related to angiogenesis and tissue remodeling, this stimulatory molecule may have a role in later phases of endothelial dysfunction and atherosclerosis. As such, the lack of an immunomodulatory effect of atorvastatin on FGF may be related to the relative lack of an important role of FGF in nonstimulated (i.e., nondysfunctional) endothelia. Granulocyte colony-stimulating factor stimulates proliferation and activation of neutrophilic cell lines. The lack of effect of atorvastatin on G-CSF concentrations may be because G-CSF is largely produced in activated macrophages and monocytes, fibroblasts, and endothelial cells. As with FGF, the low level of activation of the endothelial cells in this Example may have limited the likelihood of observing a drug effect on G-CSF. The findings herein with regard to the angiogenic factors do not preclude a drug effect on FGF, G-CSF, or other similar molecules in the setting of high-inflammatory tone, as in advanced endothelial dysfunction, atherosclerosis, or acute coronary syndromes.

The ability of atorvastatin to significantly lower IL-8, ENA-78, MCP-1, and IL-6 concentrations was reversed with the addition of mevalonate. This observation suggests that HMG-CoA reductase inhibition mediates these particular immunomodulatory effects of atorvastatin. Whether the antiinflammatory effects of statins are independent of their ability to lower cholesterol is debated. Although the cholesterol-lowering and antiinflammatory effects of statins appear somewhat disconnected, the data herein suggest that atorvastatin lowers concentrations of inflammatory mediators from endothelial cells by inhibiting the same pathway that ultimately results in cholesterol biosynthesis (i.e., the HMGCoA reductase pathway). The data herein also strongly suggest that the ability of atorvastatin to lower inflammatory chemokine and cytokine concentrations at the endothelial level is related to the depletion of mevalonate.

The data herein suggest that there is a potentially incomplete reversal of atorvastatin effects by mevalonate at the highest concentration for some markers (e.g., ENA-78 and IL-8). This finding raises the possibility that, at the highest tested concentration, atorvastatin exerts ancillary immunomodulatory effects partly independent of HMG-CoA reductase inhibition.

A dynamic range of concentrations of atorvastatin consistent with those of previous in vitro studies in endothelial and other cell systems was studied herein. Various concentrations were extrapolated to a central compartment based on previous pharmacokinetic studies and translated into atorvastatin doses of approximately 2 mg to upwards of 80 mg, a range that includes doses commonly used in clinical practice and that reduces adverse cardiovascular outcomes in secondary prevention studies.

Reduction in circulating concentrations of protein mediators of inflammation and cardiovascular disease risk have translated into clinical benefits of statins. In the present Example, protein concentrations were measured using a robust, flow-based multiplex sandwich assay that allowed for simultaneous measurements of chemokine, cytokine, and angiogenic factor concentrations. As such, this Example is the first to characterize a basal multianalyte profile for human umbilical vein endothelial cells and their response to atorvastatin in a closed quantitative system. Furthermore, this method of simultaneous protein quantification is useful in identifying patients with particular at-risk inflammatory phenotypes who might be candidates for statin therapy independent of or in conjunction with measurements of lipoprotein concentrations.

Conclusion

Atorvastatin has beneficial effects on endothelial cell inflammation, even at a low level of cellular activation. The immunomodulatory effects were concentration dependent and related to HMG-CoA reductase inhibition.

### EXAMPLE 11

### Modulation of IL-1beta Stimulated Production of CXCL5/ENA-78 By Atorvastatin in Human Endothelial Cells

Endothelial inflammation is thought to play a major role in the development and perpetuation of cardiovascular disease (CVD). The inflammatory processes that contribute to CVD pathogenesis are complex, and various cellular mediators of inflammation have been shown to contribute. Multifunctional cytokines such as interleukin-1 (IL-1) play central roles in vascular inflammation. For example, IL-1 produced from monocytes/macrophages, neutrophils, platelets, fibroblasts, and other cells can initiate inflammation by inducing the production of chemokines from endothelial cells. Chemokines, in turn, perpetuate the process through recruitment and activation of additional cellular mediators of inflammation such as monocytes and neutrophils.

Epithelial neutrophil-activating peptide 78 is a C-X-C chemokine known to be an attractor and activator of neutrophils. CXCL5 expression has been shown to be highly inducible in endothelial and vascular smooth muscle cells by IL-1beta. Additionally, there is also suggestion of a clinical tie between IL-1beta and CXCL5. Early papers identify IL-1beta as a biomarker both in congestive heart failure with progressively elevated levels with increasing New York Heart Association classification and in cerebral spinal fluid in victims of stroke, especially in patients classified as having a major stroke. More recently there have been data showing elevated levels of ENA-78 in congestive heart failure patients and in patients' cerebral spinal fluid post ischemic stroke, along with up-regulation of ENA-78's receptor's, CXCR1 and CXCR2, in heart failure patients.

The HMG-CoA reductase inhibitors (statins) have been shown to be cardioprotective, in part, through their anti-inflammatory effects. As demonstrated herein, atorvastatin decreases production of the protein product of CXCL5 (ENA-78) from quiescent endothelial cells in a dose- and mevalonate-dependent fashion. However, it is unknown whether atorvastatin has any effect on CXCL5 expression in a pro-inflammatory environment, typified by presence of IL-1beta. As such, in the following example, atorvastatin ability to modulate CXCL5 expression and ENA-78 production in endothelial cells exposed to IL-1beta was tested, where ENA-78 production in endothelial cells exposed to IL-1beta is a model of cardiovascular inflammation. This Example also characterizes the time course of atorvastatin effects on IL-1beta-stimulated ENA-78 production. Furthermore, this Example assesses whether drug effects were dependent on downstream metabolites of mevalonate in order to further elucidate the mechanism of atorvastatin's effects on CXCL5. Finally, to further investigate any statin effect on ENA-78 protein changes both alone and in conjunction with IL-1beta, the impact of atorvastatin and/or IL-1beta stimulation on gene expression was assessed.

Methods

Cell Culture and Reagents

Human umbilical vein endothelial cells (HUVECs) (Clonetics Cell Systems; Cambrex Bio Science Walkersville, Inc., Walkersville, USA) were cultured as previously described. Briefly, HUVECs between passes three and four were seeded at a density of 2.5×10⁴ cells per cm² and cultured to 80% confluence with endothelial cell growth-supplemented medium (EGM-2-endothelial cell medium-2; Cambrex Bio Science Walkersville, Inc.). Once confluent, media was changed to serum free endothelial basal media (EBM-2-endothelial cell medium-2; Cambrex Bio Science Walkersville, Inc.) for 24 hours prior to treatment, in which EBM-2 media supplemented to a 2% FBS concentration was used.

Treatments consisted of atorvastatin calcium (AT; LKT Laboratories Inc., St. Paul, USA) 1-50 μM dissolved in dimethyl sulfoxide (DMSO), IL-1beta, mevalonate, farnesyl pyrophosphate (FPP), and geranylgeranyl pyrophosphate (GGPP) were purchased from Sigma-Aldrich (St. Louis, Mo.), with concentrations of 2 ng/ml, 250 μM, 5 μM, and 5 μM used respectively. All were dissolved in molecular grade water. All final DMSO concentrations were less then 0.1%.

Treatments

Cell viability was assessed by treating HUVECs with AT 1 μM, 5 μM, 10 μM, 25 μM, and 50 μM using trypan blue cell staining. In experiments investigating the impact of AT on IL-1beta-stimulated ENA-78 production, HUVECs were pre-treated with AT followed by IL-1beta stimulation two hours later. After dose-ranging studies were performed, the time-dependent effects of AT were investigated at 0, 4, 12, 24, and 48 hours. In a separate set of experiments, cells were cultured with IL-1beta and AT in the presence or absence of mevalonate, FPP, and GGPP to determine whether AT inhibition of ENA-78 production was dependent on HMG-CoA reductase inhibition and subsequent downstream pathways. Cell culture supernates for all experiments were stored at −20° C. until analysis, which was performed within seven days.

Protein Quantification and Gene Expression

ENA-78 concentrations were measured and normalized to total protein content in cell culture supernates using cytometric immunofluorescence as previously described. Ribonucleic acid (RNA) was isolated using a commercial kit (RNeasy mini kit, Qiagen Inc., Valencia, USA). Complementary deoxyribonucleic acid (cDNA) conversion was performed by high capacity cDNA reverse transcription (Applied Biosystems, Foster City, USA) per protocol using roughly 2 μg of total RNA. RNA quality was assessed by absorbance (Nanodrop, Nanodrop Technologies, Wilmington, USA). Real-time reverse-transcription PCR (RT-PCR) was done using primer and probe sets for CXCL5 normalized to a housekeeping gene, glyceraldehyde-3-phosphate dehydrogenase (GAPDH; Taqman Gene Expression Assays, Applied Biosystems Inc.). Relative quantification was verified by gel electrophoresis of a PCR reaction using exonic primers for CXCL5 (5-CGGGAAGGAAATTTGTCTTGA-3, SEQ ID NO:7; 5-AGCTTAAGCG GCAAACATAGG-3, SEQ ID NO:8) and GAPDH (5-AAAATCAAGTGGGGCGATG CT-3, SEQ ID NO:9; 5-GCCAGGGGTGCTAAGCAGTT-3, SEQ ID NO:10). PCR for both CXCL5 and GAPDH was performed at the same time using the following conditions: 95° C. for 2 minutes; 35 cycles of 95° C. for 30 seconds, 59° C. for 30 seconds, and 72° C. for 30 seconds; followed by 7 minutes of elongation at 72° C.

Statistical Analysis

Statistical differences in normalized protein concentrations and gene expression levels were determined by one way ANOVA and post-hoc Tukey analysis. Real time gene expression was compared using log base 10 of the relative quantification, determined by the 2^{-ΔΔCt} method, of CXCL5 expression compared to control. An independent t-test was used to compare treatment differences at the same time points in the dose range analysis. A p-value<0.05 was considered significant. Statistical analyses were performed with SPSS software, version 11.5 (SPSS Inc., Chicago, USA).

Results

Effects of AT on IL-1beta-induced ENA-78 Production from Endothelial Cells

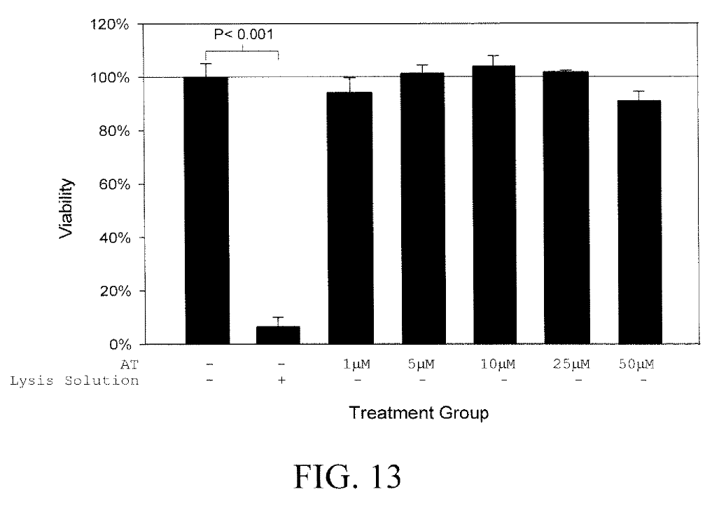

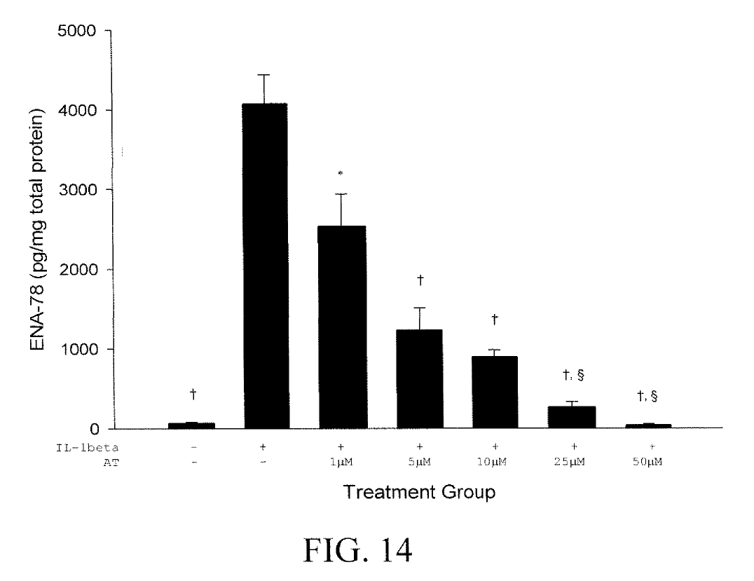

There were no significant differences in HUVEC viability comparing control and 24 hours of treatment with any dose of AT ranging from 1 uM to 50 uM (FIG. 13, Cell viability of HUVECs cultured for 24 hours with atorvastatin (AT)). Cytotoxicity was only evident for cells co-cultured with cell lysis solution. IL-1beta stimulated ENA-78 production reached nearly 60-fold higher levels than constitutive concentrations (4075±360 pg/mg vs. 69±11 pg/mg; P<1×10⁻⁴; FIG. 14, where atorvastatin (AT) attenuates interleukin-1beta (IL-1beta) induced epithelial neutrophil activating peptide-78 (ENA-78) production in a dose dependent fashion; * P<0.05 in relation to IL-1beta stimulation, † P<0.001 in relation to IL-1beta stimulation, § P>0.99 in relation to control).

Treatment with AT Attenuated IL-1beta-Stimulated Production of ENA-78 in a Dose-Dependent Fashion

ENA-78 production was reduced by 38%, 70%, 78%, 93%, 99% in the five AT dose groups compared with IL-1beta-stimulated cells in the absence of AT (P<1×10⁻⁴ for all comparisons). In fact, ENA-78 production returned to baseline when treated with AT at concentrations ≧25 uM.

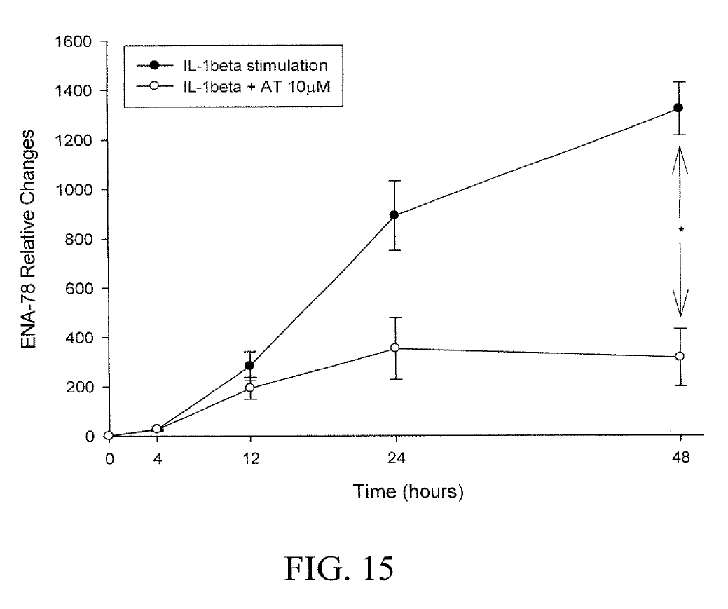

The time course of ENA-78 changes in response to IL-1beta and AT co-treatment are shown in FIG. 15, where atorvastatin (AT) attenuation of epithelial neutrophil activating peptide-78 (ENA-78) production by interleukin-1beta (IL-1beta) over time; *P<0.05. Stimulation of ENA-78 production by IL-1beta at 4, 12, 24 and 48 hours were 29, 282, 892, and 1322 times that of basal production respectively (P<1×10⁻³ for all comparisons). AT attenuation of ENA-78 production relative to IL-1beta was most pronounced at 48 hours (P<0.05).

Mevalonate and Downstream Metabolites Reverse AT Effects on IL-1beta-induced ENA-78 Production from Endothelial Cells

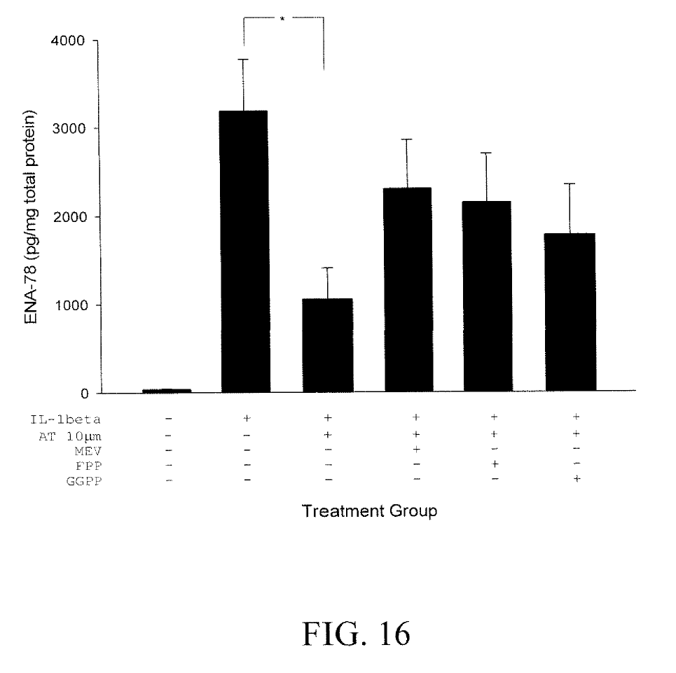

To determine whether AT effects were dependent on HMG-CoA reductase inhibition, HUVECs were culture with IL-1beta and AT 10 uM in the presence or absence of mevalonate, FPP, and GGPP (FIG. 16, where Mevalonate (MEV) and its metabolites's effects on atorvastatin (AT) in conjunction with interleukin-1beta (IL-1beta); farnesyl pyrophosphate (FPP), geranylgeranyl pyrophosphate (GGPP), * P<0.05 compared to IL-1beta stimulation alone). As expected, AT significantly attenuated IL-1beta-stimulated ENA-78 production (3181±590 pg/mg vs. 1054±349 pg/mg; P<0.05). However, co-treatment with mevalonate, FPP, or GGPP all partially reversed the effect of AT, implicating HMG-CoA reductase inhibition, farnesylation inhibition, and geranylgeranylation inhibition as potential mechanisms of AT-mediated reductions in ENA-78 production from the endothelium.

Gene Expression

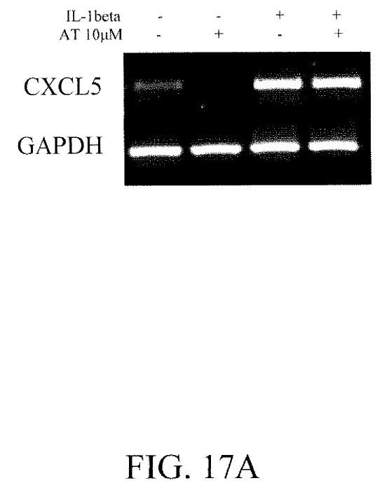

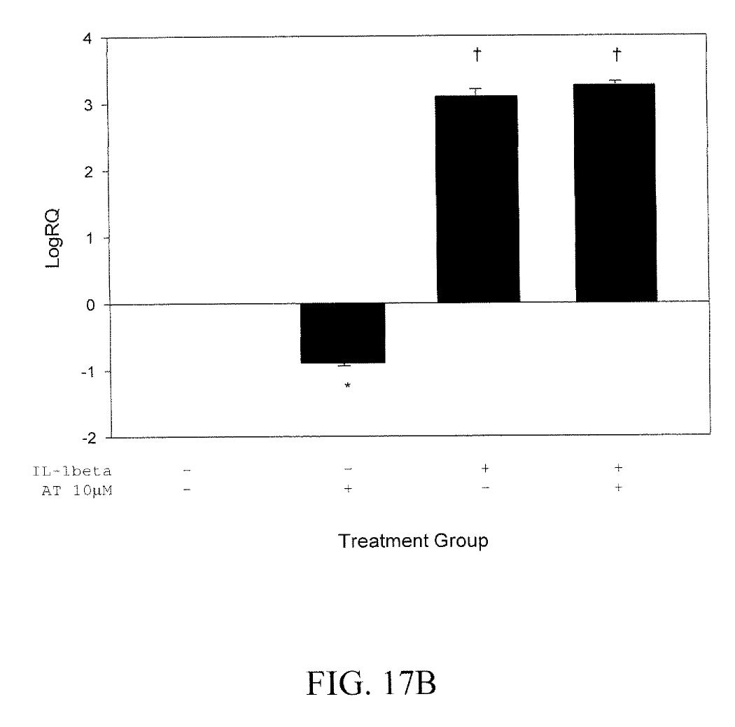

AT significantly lowered constitutive expression of CXCL5 (P=0.002) and IL-1beta highly induced CXCL5 expression in HUVECs at 24 hours (P<1×10⁻⁴). When endothelial cells were pre-treated with 10 μM of AT followed by IL-1beta in two hours, there was no difference compared to IL-1beta alone. Gel electrophoresis of PCR products of CXCL5 and GAPDH confirmed these results (FIG. 17, where CXCL5 gene expression; (A) Gel electrophoresis of PCR product of CXCL5 and GAPDH; (B) Log₁₀ Relative Quantification of CXCL5 Modulated by atorvastatin (AT); interleukin-1beta (IL-1beta), *P=0.002, † P<1×10⁻⁴).

All patents, patent applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A method for diagnosis and prediction of an inflammatory disease that is associated with abnormal levels of ENA-78 in an individual, comprising identifying whether a nucleic acid sequence exhibits at least one polymorphism in a CXCL5 gene, wherein the polymorphism(s) in an individual is indicative of said individual having a greater likelihood for an inflammatory disease than an individual without the polymorphism(s); and thereby diagnosing or predicting the inflammatory disease if the polymorphism is identified.

2. The method of claim 1 wherein the step of identifying such polymorphism comprises comparing the polymorphism in the CXCL5 gene to a normal wild type CXCL5 gene (Genbank Accession No. AF349466).

3. The method of claim 1, wherein the identifying step comprises the steps of obtaining a biological sample from the individual and testing that biological sample to identify whether a polymorphism is contained in the CXCL5 gene.

4. The method of claim 1, wherein the identifying step comprises sequencing and/or probing of the nucleic acid sequence.

5. The method of claim 4, wherein the identifying step is selected from the group consisting of: a) allele specific oligonucleotide hybridization; b) size analysis; c) sequencing; d) hybridization; e) 5′ nuclease digestion; f) single-stranded conformation polymorphism; g) allele specific hybridization; h) primer specific extension; and j) oligonucleotide ligation assay.

6. The method of claim 1, whereby the polymorphism is any one or combination selected from the group consisting of: rs425535; rs352046; rs3775488; rs352047; rs2437285; rs16850352; rs352045; rs12512838; rs454618; rs16850345; rs17813879; rs16850354; rs12505025; rs16850337; rs11551733; rs3211021; rs2437283; rs34057204; rs3211020; rs34648742; rs7693610; rs2437284; rs3864158; rs35273633; rs35811098; rs4379035; rs34160952; rs34445376; rs34721804; rs35883103; rs34249049; rs34386106; rs1540413; and rs2458099.

7. The method of claim 6, whereby the polymorphism is a substitution of cysteine for guanine at position −156 of the promoter (rs352046).

8. The method of claim 7, wherein the polymorphism is detected at position −156 of a PCR sequence using a forward primer and a reverse primer.

9. The method of claim 8, wherein the forward primer is SEQ ID NO:1 or and the reverse primer is SEQ ID NO:2.

10. The method of claim 6, whereby the polymorphism is a substitution of adenine for guanine at position 398 (rs425535).

11. The method of claim 9, wherein the polymorphism is detected at position 398 of a PCR sequence using a forward primer and a reverse primer.

12. The method of claim 11, wherein the forward primer is SEQ ID NO:3 and the reverse primer is SEQ ID NO:4.

13. The method of claim 1, wherein the inflammatory disease is selected from the group consisting of: arthritis; kidney failure; lupus; asthma; psoriasis; pancreatitis; allergy; fibrosis; anemia; and fibromyalgia.

14. The method of claim 1, wherein the inflammatory disease is selected from the group consisting of: cancer; heart; Alzheimer's disease; congestive heart failure; stroke; aortic valve stenosis; arteriosclerosis, coronary artery disease, peripheral vascular disease, osteoporosis, Parkinson's disease, inflammatory bowel disease; Crohn's disease; ulcerative colitis; multiple sclerosis; diabetes; chronic obstructive pulmonary disease; and scleroderma.

15. A method for selecting an appropriate therapeutic for an individual that has or is predisposed to developing an inflammatory disease, comprising the steps of: identifying whether the individual contains at least one polymorphism in a CXCL5 gene that is associated with abnormal levels of ENA-78 in the individual; and selecting a therapeutic that compensates for the polymorphism(s).

16. The method of claim 15, wherein said identifying step is performed using a technique selected from the group consisting of: a) allele specific oligonucleotide hybridization; b) size analysis; c) sequencing; d) hybridization; e) 5′ nuclease digestion; f) single-stranded conformation polymorphism; g) allele specific hybridization; h) primer specific extension; and j) oligonucleotide ligation assay.

17. The method of claim 15, wherein the therapeutic is selected from the group consisting of: alteration in diet, lifestyle, and exercise regimen; invasive and noninvasive surgical techniques; pharmaceutical intervention; and non-polymorphic CXCL5 gene administration.

18. The method of claim 17, wherein pharmaceutical intervention is a modulator of ENA-78 activity.

19. The method of claim 17, wherein the pharmaceutical intervention is selected from the group consisting of: statins, fibrates, ACE inhibitors, angiotensin II receptor antagonists, diuretics, alpha-adrenoreceptor antagonists, cardiac glycosides, phosphodiesterase inhibitors, beta1-adrenoreceptor antagonists, beta-2 adrenoreceptor agonists, leukotriene receptor antagonists, calcium channel blockers, HMG-CoA reductase inhibitors, bile acid sequestrants, fibric acid derivatives, thiazolidinediones, peroxisome proliferator-activated receptor agonists and antagonists, biguanides, imidazoline receptor blockers, endothelin receptor blockers, CETP inhibitors, non-steroidal anti-inflammatory agents, immunologics, and organic nitrites.

20. A method for treating an inflammatory disease that is associated with abnormal levels of ENA-78 in an individual, comprising identifying whether a nucleic acid sequence exhibits at least one polymorphism in a CXCL5 gene, wherein the polymorphism(s) in an individual is indicative of said individual having a greater likelihood for an inflammatory disease than an individual without the polymorphism(s); and administering to the individual a therapeutic that compensates for the polymorphism(s).

21. The method of claim 20, wherein the inflammatory disease is selected from the group consisting of: cancer; heart; Alzheimer's disease; congestive heart failure; stroke; aortic valve stenosis; arteriosclerosis, coronary artery disease, peripheral vascular disease, osteoporosis, Parkinson's disease, inflammatory bowel disease; Crohn's disease; ulcerative colitis; multiple sclerosis; diabetes; chronic obstructive pulmonary disease; and scleroderma.

22. The method of claim 20, wherein the identifying step is selected from the group consisting of: a) allele specific oligonucleotide hybridization; b) size analysis; c) sequencing; d) hybridization; e) 5′ nuclease digestion; f) single-stranded conformation polymorphism; g) allele specific hybridization; h) primer specific extension; and j) oligonucleotide ligation assay.

23. The method of claim 20, wherein the therapeutic is selected from the group consisting of: alteration in diet, lifestyle, and exercise regimen; invasive and noninvasive surgical techniques; and pharmaceutical intervention.

24. The method of claim 23, wherein pharmaceutical intervention is a modulator of ENA-78 activity.

25. The method of claim 23, wherein the therapeutic is selected from the group consisting of: statins, fibrates, ACE inhibitors, angiotensin II receptor antagonists, diuretics, alpha-adrenoreceptor antagonists, cardiac glycosides, phosphodiesterase inhibitors, beta1-andrenoreceptor antagonists, beta-2 adrenoreceptor agonists, leukotriene receptor antagonists, calcium channel blockers, HMG-CoA reductase inhibitors, bile acid sequestrants, fibric acid derivatives, thiazolidinediones, peroxisome proliferator-activated receptor agonists and antagonists, biguanides, imidazoline receptor blockers, endothelin receptor blockers, CETP inhibitors, non-steroidal anti-inflammatory agents, immunologics, and organic nitrites.

26. A method for identifying an individual that will respond to anti-inflammatory treatment comprising the steps of: identifying whether the individual contains at least one polymorphism in a CXCL5 gene that is associated with abnormal levels of ENA-78 in the individual; and selecting a therapeutic that compensates for the polymorphism(s).
